(19) United States
(12) Patent Application Publication
(10) Pub. No.: US 2025/0333808 A1
(43) Pub. Date: Oct. 30, 2025

(54) PROBES FOR IMPROVING ENVIRONMENTAL SAMPLE SURVEILLANCE

(71) Applicant: ILLUMINA, INC., San Diego, CA (US)

(72) Inventors: Brian Hawks, San Diego, CA (US); Stephen Gross, San Diego, CA (US); Gary Schroth, Alamo, CA (US); Rachel Adams, El Cerrito, CA (US); Keith Arora-Williams, Laguna Nigel, CA (US); Kate Broadbent, Salt Lake City, UT (US)

(21) Appl. No.: 18/987,420

(22) Filed: Dec. 19, 2024

Related U.S. Application Data
(63) Continuation of application No. PCT/US2023/076171, filed on Oct. 6, 2023.
(60) Provisional application No. 63/378,636, filed on Oct. 6, 2022.
(60) Provisional application No. 63/479,827, filed on Jan. 13, 2023.
(60) Provisional application No. 63/480,862, filed on Jan. 20, 2023.

(30) Foreign Application Priority Data
Oct. 6, 2023 (WO) PCT/US2023/076171

(51) Int. Cl. C12Q 1/70; C12N 15/10; C12Q 1/6806; C12Q 1/6869

(52) U.S. Cl. CPC C12Q 1/701; C12N 15/1006; C12N 15/1065; C12N 15/1096; C12Q 1/6806; C12Q 1/6869; C12Q 2600/16

(57) ABSTRACT

Described herein are compositions and methods for enriching library fragments comprising viral sequences prepared from a variety of samples. These methods may incorporate microfluidics and flowcells for greater ease of use. Libraries enriched with the present methods may be used for sequencing. Also described are probes and methods for enzymatic depletion of unwanted RNA.

### RELATED APPLICATIONS

This application is a bypass continuation of PCT/2023/076171, filed on Oct. 6, 2023. PCT/2023/076171 claims priority to U.S. provisional application 63/378,636 filed on Oct. 6, 2023; U.S. provisional application 63/479,827 filed on Jan. 13, 2023; and U.S. provisional application 63/480,862 filed on Jan. 20, 2023. Each application is incorporated herein by reference in its entirety.

### REFERENCE TO ELECTRONIC SEQUENCE LISTING

The application contains a Sequence Listing that has been submitted on a Read-Only Optical Disc in .XML format and is hereby incorporated by reference in its entirety. Said .XML file, created on Jan. 4, 2024, is named “IP-2397-US SL” and is 209,829 KB in size. The Sequence Listing is on a Read-Only Optical Disc created on Mar. 11, 2025. The sequence listing contained in this .XML file is part of the specification and is hereby incorporated by reference herein in its entirety.

### DESCRIPTION

### Field

This disclosure relates to probes for improving environmental sample (including wastewater samples and other samples) surveillance and surveillance of other samples for various viruses. Libraries enriched with the present methods may be used to generate sequencing data. Also described are viral probes and methods for viral probe design and for enzymatic depletion of unwanted RNA and cDNA from human wastewater and other samples.

### BACKGROUND

Viruses continue to develop naturally resulting in new strains and diseases to human populations. For example, the World Health Organization (WHO) declared infection by the novel Severe Acute Respiratory Syndrome Coronavirus 2 (SARS-COV-2) as a pandemic and termed the related disease as coronavirus disease 2019 (COVID-19). SARS-COV-2 can be detected in feces. Additionally, most persons infected with enterically transmitted viruses shed large amounts of virus in feces for days or weeks, both before and after onset of symptoms. Therefore, viruses causing gastroenteritis may be detected in wastewater, even if only a few persons are infected. The abundance and diversity of pathogenic viruses in wastewater has been shown to reflect the pattern of infection in human population. Adenovirus (HAdV), rotavirus (RoV), hepatitis A virus (HAV), and other enteric viruses, such as norovirus (NoV), coxsackievirus, echovirus, reovirus and astrovirus are some of the principal human pathogenic viruses transmissible via water media.

Viruses are ubiquitous and persistent in raw wastewater and treated wastewater. One of the main sources of viruses, including viral pathogens in wastewater is human fecal matter, particularly that from infected persons. Sewage systems receive enteric viruses excreted by infected individuals. In addition to human pathogenic viruses, waterborne viruses that originate from food production, animal husbandry, seasonal surface runoff and other sources are present in wastewater. Wastewater can serve as a significant source of information for public health and agricultural officials on the pathogens present in a population and the levels of those pathogens.

The bodies that receive treated wastewater are oftentimes used for recreational activities and agriculture, and as a source of raw water for drinking water production. The presence of potentially pathogenic viruses in wastewater is of concern since it can pose risks to human health. While this presents an opportunity to investigate wastewater for incidence of disease or presence of potentially pathogenic viruses, sampling and measuring wastewater for a virus-of-interest is problematic due to low concentrations of this virus or particles thereof alone. The mixture of contaminants (e.g., other waterborne pathogens including bacterial, fungal, and parasitic pathogens, as well as viruses not of interest or human nucleic acids) and a virus-of-interest presents a difficult medium for viral DNA and RNA extraction therefrom, especially where concentrations of a virus-of-interest are low. As such, methods of enriching wastewater samples for viral targets are needed to quantify incidence of viral infection or disease in a community and to identify novel viruses of interest in wastewater, such as from a sewer system, and methods of recovering nucleic acids from a virus-of-interest in wastewater. Public health officials also need methods of recovering nucleic acids from a virus-of-interest in wastewater. Investigations of other types of samples would also benefit from improved methods of recovering nucleic acids.

Described herein is the development of a viral probe set for enrichment and detection of novel strains or variants of genetically related viruses. Through an iterative design process, the viral probes described herein are optimized to capture a broad diversity of viral sequences to increase the chance of capturing genomic sequence from a yet to be discovered strain or novel variant coronavirus or other virus-of-interest. The viral probe set and viral probe design methods described herein minimize probe redundancy to reduce the overall number of oligonucleotides that are necessary to detect such a broad diversity of viral sequences.

### SUMMARY

In accordance with the description, described herein are methods of enriching a sample for one or more virus-of-interest nucleic acids and/or for improving environmental wastewater surveillance for various viruses. These methods may be performed with standard lab equipment, such as flowcells comprised in sequencers. In some embodiments, standard sequencing consumables and platform (i.e., sequencer) can be used as a microfluidic device for enriching and/or depleting library fragments. In some embodiments, depleting abundant small noncoding RNA is performed after cDNA synthesis and amplification.

Embodiment 1. A method of enriching a sample for one or more target viral nucleic acids comprising the steps of: (a) providing a probe set comprising at least two nucleic acid probes complementary to one or more target viral nucleic acids, wherein the probe set comprises at least two of SEQ ID NOs: 1-213,280, or its complement; (b) allowing the probes in the probe set to hybridize to the target viral nucleic acids; (c) enriching the sample for the one or more target viral nucleic acids by amplifying the target viral nucleic acids and/or separating the target viral nucleic acids from the sample.

Embodiment 2. A method of enriching a sample for one or more target viral nucleic acids comprising the steps of: (a) providing a probe set comprising at least two nucleic acid probes complementary to one or more target viral nucleic acids, wherein the nucleic acid probes are affixed to a support; (b) capturing the one or more target viral nucleic acids on the support; (c) using the one or more captured target viral nucleic acids as a template strand to produce one or more nucleic acid duplexes immobilized on the support, wherein one or more target viral nucleic acids hybridize to one or more probes of the probe set on the support; (d) contacting a transposase and transposon with the one or more nucleic acid duplexes under conditions wherein the one or more nucleic acid duplexes and transposon composition undergo a transposition reaction to produce one or more tagged nucleic acid duplexes, wherein the transposon composition comprises a double stranded nucleic acid molecule comprising a transferred strand and a non-transferred strand; (e) contacting the one or more tagged nucleic acid duplexes with a nucleic acid modifying enzyme under conditions to extend the 3′ end of the immobilized strand to the 5′ end of the template strand to produce one or more end-extended tagged nucleic acid duplexes; (f) amplifying the one or more end-extended tagged nucleic acid duplexes to produce a plurality of tagged nucleic acid strands; (g) contacting the plurality of tagged nucleic acid strands with a probe set to create an enriched library; and (h) amplifying the enriched library.

Embodiment 3. The method of embodiment 1 or 2, wherein the sample comprises a sample from a mammal.

Embodiment 4. The method of embodiment 3, wherein the sample comprises a sample from a human, monkey, bat, dog, cat, horse, goat, sheep, cow, pig, rat and/or mouse.

Embodiment 5. The method of any one of embodiments 1-4, wherein the sample comprises a blood sample, a serum sample, and/or a whole blood sample.

Embodiment 6. The method of any one of embodiments 1-4, wherein the sample comprises a tissue sample.

Embodiment 7. The method of any one of embodiments 1-4, wherein the sample comprises a fecal sample, a urine sample, a mucus sample, a saliva sample, a lymph sample, a vaginal fluid sample, a semen sample, an amniotic sample, and/or a sweat sample.

Embodiment 8. The method of embodiment 1 or 2, comprises a freshwater sample, a wastewater sample, a saline water sample, or a combination thereof.

Embodiment 9. The method of embodiment 8, wherein the sample comprises a wastewater sample.

Embodiment 10. The method of any one of embodiments 1-9, wherein the probe set is biotinylated.

Embodiment 11. The method of any one of embodiments 1-10, wherein the one or more target nucleic acids are viral RNA molecules.

Embodiment 12. The method of any one of embodiments 1-11, wherein the one or more target nucleic acids are genomic viral DNA or RNA molecules.

Embodiment 13. The method of any one of embodiments 1-12, wherein the probe set further comprises at least two DNA probes that each hybridize to at least one target virus molecule from an adenovirus, Aichivirus, Andes virus, Anjozorobe hantavirus, Araraquara virus, Bayou virus, Bermejo virus, Black Creek Canal virus, Castelo dos Sonhos virus, Chapare virus, Chikungunya virus, Choclo virus, coxsackievirus, Crimean-Congo haemorrhagic fever virus, Dengue virus, Dobrava virus, Eastern equine encephalitis virus, Ebola virus, enterovirus, Guanarito virus, Hantaan virus, Hendra virus, hepatitis A virus, hepatitis B virus, hepatitis C virus, human coronavirus, human immunodeficiency virus 1, human immunodeficiency virus 2, human metapneumovirus, human papillomavirus, influenza A virus, influenza B virus, Japanese encephalitis virus, Juquitiba virus, KI polyomavirus Stockholm 60, Kyasanur forest disease virus, Laguna Negra virus, Lassa virus, Lechiguanas virus, Lujo virus, Machupo virus, Maciel virus, Marburg virus, Merkel cell polyomavirus, Middle East respiratory syndrome-related coronavirus, monkeypox virus, Monongahela hantavirus, Mopeia Lassa virus, Nipah virus, norovirus, Omsk hemorrhagic fever virus, orthohantavirus, parainfluenza, parechovirus, parvovirus, polyomavirus, Puumala virus, respiratory syncytial virus, rhinovirus A, rhinovirus B, rhinovirus C, Rift Valley fever, Rio Mamore virus, rotavirus A, rotavirus B, rotavirus B, rotavirus C, rotavirus H, rubella virus, Saaremaa virus, Sabia virus, salivirus, Sangassou virus, sapovirus, SARS coronavirus, Seoul virus, sin nombre virus, tick-borne encephalitis virus, torque teno virus, Tula virus, variola virus, Venezuelan equine encephalitis virus, West Nile virus, Western equine encephalomyelitis virus, yellow fever virus, and/or Zika virus.

Embodiment 14. The method of any one of embodiments 1-13, wherein the probe set further comprises at least two DNA probes that each hybridize to at least one target virus molecule selected from Table 2.

Embodiment 15. The method of any one of embodiments 1-14, wherein the probe set further comprises at least two DNA probes that each hybridize to at least one target virus molecule selected from Adeno-associated virus 2 (AAV2), Aichi virus 1 (AiV-A1), Alkhumra hemorrhagic fever virus (AHFV), Andes virus (ANDV), Anjozorobe virus (ANJV), Araucaria virus, Australian bat lyssavirus (ABLV), Bayou virus (BAYV), BK polyomavirus (BKPyV), Black Creek Canal virus (BCCV), Bombali virus (BOMV), Bourbon virus (BRBV), Bundibugyo virus (BDBV), Cache Valley virus (CVV), California encephalitis virus (CEV), Cedar virus (CedV), Chapare virus (CHAPV), Chikungunya virus (CHIKV), Choclo virus (CHOV), Colorado tick fever virus (CTFV), Crimean-Congo hemorrhagic fever virus (CCHFV), Crimean-Congo hemorrhagic fever virus 2 (CCHFV-2), Dengue virus (DENV), Dobrava-Belgrade virus (DOBV), Duvenhage virus (DUVV), Eastern equine encephalitis virus (EEEV), Ebola virus (EBOV), Enterovirus A, Enterovirus B, Enterovirus C, Enterovirus D, Epstein-Barr virus (EBV), European bat lyssavirus (EBLV), Ghana virus (GhV), Guanarito virus (GTOV), Hantaan virus (HTNV), Heartland virus (HRTV), Hendra virus (HeV), Henipavirus unclassified, Hepatitis A virus (HAV), Hepatitis B virus (HBV), Hepatitis C virus (HCV), Hepatitis D virus (HDV), Hepatitis E virus (HEV), Herpes simplex virus 1 (HSV1), Herpes simplex virus 2 (HSV2), Human adenovirus A, Human adenovirus B, Human adenovirus C, Human adenovirus D, Human adenovirus E, Human adenovirus F, Human adenovirus G, Human bocavirus (HBoV), Human coronavirus 229E (HCOV_229E), Human coronavirus HKU1 (HCOV_HKU1), Human coronavirus NL63 (HCoV_NL63), Human coronavirus OC43 (HCoV_OC43), Human cytomegalovirus (HCMV), Human immunodeficiency virus 1 (HIV-1), Human immunodeficiency virus 2 (HIV-2), Human metapneumovirus (HMPV), Human papillomavirus 11 (HPV11), Human papillomavirus 16 (HPV16; high-risk), Human papillomavirus 18 (HPV18; high-risk), Human papillomavirus 26 (HPV26), Human papillomavirus 31 (HPV31; high-risk), Human papillomavirus 33 (HPV33; high-risk), Human papillomavirus 35 (HPV35; high-risk), Human papillomavirus 39 (HPV39; high-risk), Human papillomavirus 40 (HPV40), Human papillomavirus 42 (HPV42), Human papillomavirus 43 (HPV43), Human papillomavirus 44 (HPV44), Human papillomavirus 45 (HPV45; high-risk), Human papillomavirus 51 (HPV51; high-risk), Human papillomavirus 52 (HPV52; high-risk), Human papillomavirus 53 (HPV53), Human papillomavirus 54 (HPV54), Human papillomavirus 56 (HPV56; high-risk), Human papillomavirus 58 (HPV58; high-risk), Human papillomavirus 59 (HPV59; high-risk), Human papillomavirus 6 (HPV6), Human papillomavirus 61 (HPV61), Human papillomavirus 66 (HPV66; high-risk), Human papillomavirus 68 (HPV68; high-risk), Human papillomavirus 69 (HPV69), Human papillomavirus 70 (HPV70), Human papillomavirus 73 (HPV73), Human papillomavirus 82 (HPV82), Human parainfluenza virus 1 (HPIV-1), Human parainfluenza virus 2 (HPIV-2), Human parainfluenza virus 3 (HPIV-3), Human parainfluenza virus 4 (HPIV-4), Human parechovirus (HPeV), Human parvovirus B19 (B19V), Human polyomavirus 6 (HPyV6), Human polyomavirus 7 (HPyV7), Human polyomavirus 9 (HPyV9), Human respiratory syncytial virus A (HRSV-A), Human respiratory syncytial virus B (HRSV-B), Influenza A virus, Influenza B virus, Influenza C virus, Isla Vista virus, Itapua virus, Jamestown Canyon virus (JCV), Japanese encephalitis virus (JEV), JC polyomavirus (JCPyV), Junin virus (JUNV), Juquitiba virus, KI polyomavirus (KIPyV), Kyasanur Forest disease virus (KFDV), La Crosse virus (LACV), Lagos bat virus (LBV), Laguna Negra virus (LANV), Langya virus, Lassa virus (LASV), LI polyomavirus (LIPyV), Lloviu virus (LLOV), Lujo virus (LUJV), Luxi virus (LUXV), Lymphocytic choriomeningitis virus (LCMV), Machupo virus (MACV), Mamastrovirus 1 (MAstV1), Mamastrovirus 6 (MAstV6), Mamastrovirus 8 (MAstV8), Mamastrovirus 9 (MAstV9), Maporal virus (MAPV), Marburg virus (MARV), Mayaro virus (MAYV), Measles virus (MV), Menangle virus (MenV), Merkel cell polyomavirus (MCPyV), Middle East respiratory syndrome-related coronavirus (MERS-COV), Mojiang virus (MojV), Mokola virus (MOKV), Monkeypox virus (MPV), Monongahela hantavirus, Muleshoe virus, Mumps virus (MuV), Murray Valley encephalitis virus (MVEV), MW polyomavirus (MWPyV), New Jersey polyomavirus (NJPyV), Nipah virus (NiV), Norovirus, Omsk hemorrhagic fever virus (OHFV), Onyong-nyong virus (ONNV), Oropouche virus (OROV), Paranoa virus, Powassan virus (POWV), Punta Toro virus (PTV), Puumala virus (PUUV), Rabies virus (RABV), Ravn virus (RAVV), Reston virus (RESTV), Rhinovirus A (RV-A), Rhinovirus B (RV-B), Rhinovirus C (RV-C), Rift Valley fever virus (RVFV), Ross River virus (RRV), Rotavirus A (RVA), Rotavirus B (RVB), Rotavirus C (RVC), Rubella virus (RuV), Sabia virus (SBAV), Salivirus A (SaV-A), Sandfly fever Sicilian virus (SFCV), Sangassou virus (SANGV), Sapovirus, Semliki Forest virus (SFV), Seoul virus (SEOV), Severe acute respiratory syndrome coronavirus (SARS-COV), Severe acute respiratory syndrome coronavirus 2 (SARS-COV-2), Severe fever with thrombocytopenia syndrome virus (SFTSV), Simian virus 40 (SV40), Sin nombre virus (SNV), Sindbis virus (SINV), Snowshoe hare virus (SSHV), Sosuga virus (SoRV), St. Louis encephalitis virus (SLEV), STL polyomavirus (STLPyV), Sudan virus (SUDV), Tacheng tick virus 2 (TcTV-2), Tahyna virus (TAHV), Tai Forest virus (TAFV), Tick-borne encephalitis virus (TBEV), Torque teno virus (TTV), Toscana virus (TOSV), Trichodysplasia spinulosa-associated polyomavirus (TSPyV), Tula virus (TULV), Usutu virus (USUV), Varicella-zoster virus (VZV), Variola virus (VARV), Venezuelan equine encephalitis virus (VEEV), West Nile virus (WNV), Western equine encephalitis virus (WEEV), WU polyomavirus (WUPyV), Yellow fever virus (YFV), and Zika virus (ZIKV).

Embodiment 16. The method of any one of embodiments 1-15, wherein the DNA probes further comprise any one of SEQ ID NOs: 213,288-213,747, or its complement.

Embodiment 17. The method of any one of embodiments 1-16, wherein the DNA probes further comprise two or more, or five or more, or 10 or more, or 25 or more sequences, or all of the sequences selected from SEQ ID NOs: 213,288-213,747, or its complement.

Embodiment 18. The method of any one of embodiments 1-17, wherein the method further comprises depleting unwanted nucleic acid molecules from a nucleic acid sample.

Embodiment 19. The method of embodiment 18, wherein the depleting unwanted nucleic acid molecules comprises depleting unwanted cDNA library fragments from a library of cDNA fragments prepared from RNA, wherein the unwanted library fragments comprise those prepared from unwanted RNA sequences, further comprising: (a) preparing a solid support comprising at least one immobilized oligonucleotide, wherein each immobilized oligonucleotide comprises a nucleic acid sequence corresponding to an unwanted RNA sequence or its complement; (b) adding the library of fragments to the solid support and hybridizing the library fragments to at least one immobilized oligonucleotide to allow binding of unwanted library fragments to at least one immobilized oligonucleotide, and (c) collecting library fragments not bound to at least one immobilized oligonucleotide.

Embodiment 20. The method of embodiment 19, wherein the at least one immobilized oligonucleotide comprises a sequence comprising any one or more of SEQ ID NOs: 213,288-214,878 or its complement.

Embodiment 21. The method of embodiment 20, wherein depleting unwanted nucleic acid molecules comprises depleting off-target RNA nucleic acid molecules from a nucleic acid sample comprises: (a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid, wherein the off-target DNA comprises at least one small noncoding RNA chosen from RN7SK, RN7SL1, RN7SL2, RN7SL5P, RPPH1, SNORD3A; (b) contacting the DNA:RNA hybrids with a ribonuclease that degrades the RNA from the DNA:RNA hybrids, thereby degrading the off-target RNA molecules in the nucleic acid sample to form a degraded mixture; (c) separating the degraded RNA from the degraded mixture; (d) sequencing the remaining RNA from the sample; (e) evaluating the remaining RNA sequences for the presence of off-target RNA molecules from the first species, thereby determining gap sequence regions; and (f) supplementing the probe set with additional DNA probes complementary to discontiguous sequences in one or more of the gap sequence regions.

Embodiment 22. The method of embodiment 21, wherein the probe set comprises any one or more of SEQ ID NOs: 213,288-213,878, or its complement.

Embodiment 23. The method of any one of embodiments 1-22, wherein the method further comprises depleting unwanted cDNA library fragments from a library of cDNA fragments prepared from RNA, wherein the unwanted library fragments comprise those prepared from unwanted RNA sequences.

Embodiment 24. A composition comprising a probe set comprising at least two DNA probes complementary to at least one target viral nucleic acid molecule in a nucleic acid sample wherein the target viral nucleic acid comprises at least one molecule selected from Table 2.

Embodiment 25. A composition comprising a probe set comprising at least two DNA probes complementary to at least one target viral nucleic acid molecule in a nucleic acid sample wherein the target viral nucleic acid comprises at least one molecule selected from Adeno-associated virus 2 (AAV2), Aichi virus 1 (AiV-A1), Alkhumra hemorrhagic fever virus (AHFV), Andes virus (ANDV), Anjozorobe virus (ANJV), Araucaria virus, Australian bat lyssavirus (ABLV), Bayou virus (BAYV), BK polyomavirus (BKPyV), Black Creek Canal virus (BCCV), Bombali virus (BOMV), Bourbon virus (BRBV), Bundibugyo virus (BDBV), Cache Valley virus (CVV), California encephalitis virus (CEV), Cedar virus (CedV), Chapare virus (CHAPV), Chikungunya virus (CHIKV), Choclo virus (CHOV), Colorado tick fever virus (CTFV), Crimean-Congo hemorrhagic fever virus (CCHFV), Crimean-Congo hemorrhagic fever virus 2 (CCHFV-2), Dengue virus (DENV), Dobrava-Belgrade virus (DOBV), Duvenhage virus (DUVV), Eastern equine encephalitis virus (EEEV), Ebola virus (EBOV), Enterovirus A, Enterovirus B, Enterovirus C, Enterovirus D, Epstein-Barr virus (EBV), European bat lyssavirus (EBLV), Ghana virus (GhV), Guanarito virus (GTOV), Hantaan virus (HTNV), Heartland virus (HRTV), Hendra virus (HeV), Henipavirus unclassified, Hepatitis A virus (HAV), Hepatitis B virus (HBV), Hepatitis C virus (HCV), Hepatitis D virus (HDV), Hepatitis E virus (HEV), Herpes simplex virus 1 (HSV1), Herpes simplex virus 2 (HSV2), Human adenovirus A, Human adenovirus B, Human adenovirus C, Human adenovirus D, Human adenovirus E, Human adenovirus F, Human adenovirus G, Human bocavirus (HBoV), Human coronavirus 229E (HCOV_229E), Human coronavirus HKU1 (HCOV_HKU1), Human coronavirus NL63 (HCOV_NL63), Human coronavirus OC43 (HCoV_OC43), Human cytomegalovirus (HCMV), Human immunodeficiency virus 1 (HIV-1), Human immunodeficiency virus 2 (HIV-2), Human metapneumovirus (HMPV), Human papillomavirus 11 (HPV11), Human papillomavirus 16 (HPV16; high-risk), Human papillomavirus 18 (HPV18; high-risk), Human papillomavirus 26 (HPV26), Human papillomavirus 31 (HPV31; high-risk), Human papillomavirus 33 (HPV33; high-risk), Human papillomavirus 35 (HPV35; high-risk), Human papillomavirus 39 (HPV39; high-risk), Human papillomavirus 40 (HPV40), Human papillomavirus 42 (HPV42), Human papillomavirus 43 (HPV43), Human papillomavirus 44 (HPV44), Human papillomavirus 45 (HPV45; high-risk), Human papillomavirus 51 (HPV51; high-risk), Human papillomavirus 52 (HPV52; high-risk), Human papillomavirus 53 (HPV53), Human papillomavirus 54 (HPV54), Human papillomavirus 56 (HPV56; high-risk), Human papillomavirus 58 (HPV58; high-risk), Human papillomavirus 59 (HPV59; high-risk), Human papillomavirus 6 (HPV6), Human papillomavirus 61 (HPV61), Human papillomavirus 66 (HPV66; high-risk), Human papillomavirus 68 (HPV68; high-risk), Human papillomavirus 69 (HPV69), Human papillomavirus 70 (HPV70), Human papillomavirus 73 (HPV73), Human papillomavirus 82 (HPV82), Human parainfluenza virus 1 (HPIV-1), Human parainfluenza virus 2 (HPIV-2), Human parainfluenza virus 3 (HPIV-3), Human parainfluenza virus 4 (HPIV-4), Human parechovirus (HPeV), Human parvovirus B19 (B19V), Human polyomavirus 6 (HPyV6), Human polyomavirus 7 (HPyV7), Human polyomavirus 9 (HPyV9), Human respiratory syncytial virus A (HRSV-A), Human respiratory syncytial virus B (HRSV-B), Influenza A virus, Influenza B virus, Influenza C virus, Isla Vista virus, Itapua virus, Jamestown Canyon virus (JCV), Japanese encephalitis virus (JEV), JC polyomavirus (JCPyV), Junin virus (JUNV), Juquitiba virus, KI polyomavirus (KIPyV), Kyasanur Forest disease virus (KFDV), La Crosse virus (LACV), Lagos bat virus (LBV), Laguna Negra virus (LANV), Langya virus, Lassa virus (LASV), LI polyomavirus (LIPyV), Lloviu virus (LLOV), Lujo virus (LUJV), Luxi virus (LUXV), Lymphocytic choriomeningitis virus (LCMV), Machupo virus (MACV), Mamastrovirus 1 (MAstV1), Mamastrovirus 6 (MAstV6), Mamastrovirus 8 (MAstV8), Mamastrovirus 9 (MAstV9), Maporal virus (MAPV), Marburg virus (MARV), Mayaro virus (MAYV), Measles virus (MV), Menangle virus (MenV), Merkel cell polyomavirus (MCPyV), Middle East respiratory syndrome-related coronavirus (MERS-COV), Mojiang virus (MojV), Mokola virus (MOKV), Monkeypox virus (MPV), Monongahela hantavirus, Muleshoe virus, Mumps virus (MuV), Murray Valley encephalitis virus (MVEV), MW polyomavirus (MWPyV), New Jersey polyomavirus (NJPyV), Nipah virus (NiV), Norovirus, Omsk hemorrhagic fever virus (OHFV), Onyong-nyong virus (ONNV), Oropouche virus (OROV), Paranoa virus, Powassan virus (POWV), Punta Toro virus (PTV), Puumala virus (PUUV), Rabies virus (RABV), Ravn virus (RAVV), Reston virus (RESTV), Rhinovirus A (RV-A), Rhinovirus B (RV-B), Rhinovirus C (RV-C), Rift Valley fever virus (RVFV), Ross River virus (RRV), Rotavirus A (RVA), Rotavirus B (RVB), Rotavirus C (RVC), Rubella virus (RuV), Sabia virus (SBAV), Salivirus A (SaV-A), Sandfly fever Sicilian virus (SFCV), Sangassou virus (SANGV), Sapovirus, Semliki Forest virus (SFV), Seoul virus (SEOV), Severe acute respiratory syndrome coronavirus (SARS-COV), Severe acute respiratory syndrome coronavirus 2 (SARS-COV-2), Severe fever with thrombocytopenia syndrome virus (SFTSV), Simian virus 40 (SV40), Sin nombre virus (SNV), Sindbis virus (SINV), Snowshoe hare virus (SSHV), Sosuga virus (SoRV), St. Louis encephalitis virus (SLEV), STL polyomavirus (STLPyV), Sudan virus (SUDV), Tacheng tick virus 2 (TcTV-2), Tahyna virus (TAHV), Tai Forest virus (TAFV), Tick-borne encephalitis virus (TBEV), Torque teno virus (TTV), Toscana virus (TOSV), Trichodysplasia spinulosa-associated polyomavirus (TSPyV), Tula virus (TULV), Usutu virus (USUV), Varicella-zoster virus (VZV), Variola virus (VARV), Venezuelan equine encephalitis virus (VEEV), West Nile virus (WNV), Western equine encephalitis virus (WEEV), WU polyomavirus (WUPyV), Yellow fever virus (YFV), and Zika virus (ZIKV).

Embodiment 26. A composition comprising a probe set comprising at least one DNA probe comprising at least one sequence of SEQ ID NOs: 1-213,280, or its complement.

Embodiment 27. The composition of any one of embodiments 25-26, comprising at least 5, at least at least 10, at least 50, at least 100, at least 250, at least 500, at least 750, at least 1000, at least 1500, or at least 2000 sequences of SEQ ID NOs: 1-213,280, or its complement.

Embodiment 28. The compositions of embodiments 25-27, further comprising at least one DNA probe comprising at least one sequence comprising at least one of SEQ ID NOs: 213,288-214,878, or its complement.

Embodiment 29. A kit comprising a probe set comprising: (a) at least one DNA probe comprising at least one sequence comprising at least one of SEQ ID NOs: 1-213,280, or its complement; (b) a buffer.

Embodiment 30. The kit of embodiments 29, further comprising at least one DNA probe comprising at least one sequence comprising at least one of SEQ ID NOs: 213,288-214,878, or its complement.

Embodiment 31. The kit of embodiments 29 and 30, wherein the buffer is a wash buffer and/or an elution buffer.

Embodiment 32. The kit of embodiment 29-31, further comprising an RNA depletion buffer, a probe depletion buffer, and/or a probe removal buffer.

Embodiment 33. The kit of any of one embodiments 29-32, further comprising: (a) a ribonuclease; (b) a DNase; and (c) RNA purification beads.

Embodiment 34. The kit of embodiment 33, wherein the ribonuclease is RNase H.

Embodiment 35. The kit of any of one embodiments 29-34, comprising a buffer and nucleic acid purification medium.

Embodiment 36 The kit of embodiment 35, wherein the buffer is an RNA depletion buffer, a probe depletion buffer, and/or a probe removal buffer.

Embodiment 37. The kit of any one of embodiments 28-34, further comprising a nucleic acid destabilizing chemical.

Embodiment 38. The kit of embodiment 35, wherein the nucleic acid destabilizing chemical comprises betaine, DMSO, formamide, glycerol, or a derivative thereof, or a mixture thereof.

Embodiment 39. The kit of any one of embodiments 35-36, wherein the nucleic acid destabilizing chemical comprises formamide.

Embodiment 40. The kit of any one of embodiments 29-39, wherein the at least one DNA probe comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, or 213,280 probes comprising sequences selected from SEQ ID NOs: 1-213,280, or its complement.

Embodiment 41. The kit of any one of embodiments 28-38, wherein the at least one DNA probe comprises 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more, 200000 or more, or 213,280 probes comprising sequences selected from SEQ ID NOs: 1-213,280, or its complement.

Additional objects and advantages will be set forth in part in the description which follows, and in part will be understood from the description, or may be learned by practice. The objects and advantages will be realized and attained by means of the elements and combinations particularly pointed out in the appended claims.

DESCRIPTION OF SEQUENCES

| | SEQ ID | |
|---|---|---|
| Description | NO: | Sequence (3′ to 5′) |
| RN7SK | 213281 | GATGTGAGGGCGATCTGGCTGCGACATCTGTCACCCCATTGATCGCCAGGG |
| | | TTGATTCGGCTGATCTGGCTGGCTAGGCGGGTGTCCCCTTCCTCCCTCACC |
| | | GCTCCATGTGCGTCCCTCCCGAAGCTGCGCGCTCGGTCGAAGAGGACGACC |
| | | ATCCCCGATAGAGGAGGACCGGTCTTCGGTCAAGGGTATACGAGTAGCTGC |
| | | GCTCCCCTGCTAGAACCTCCAAACAAGCTCTCAAGGTCCATTTGTAGGAGA |
| | | ACGTAGGGTAGTCAAGCTTCCAAGACTCCAGACACATCCAAATGAGGCGCT |
| | | GCATGTGGCAGTCTGCCTTTCT |
| RN7SL1 | 213282 | GCCGGGCGCGGTGGCGCGTGCCTGTAGTCCCAGCTACTCGGGAGGCTGAGG |
| | | CTGGAGGATCGCTTGAGTCCAGGAGTTCTGGGCTGTAGTGCGCTATGCCGA |
| | | TCGGGTGTCCGCACTAAGTTCGGCATCAATATGGTGACCTCCCGGGAGCGG |
| | | GGGACCACCAGGTTGCCTAAGGAGGGGTGAACCGGCCCAGGTCGGAAACGG |
| | | AGCAGGTCAAAACTCCCGTGCTGATCAGTAGTGGGATCGCGCCTGTGAATA |
| | | GCCACTGCACTCCAGCCTGGGCAACATAGCGAGACCCCGTCTCT |
| RN7SL2 | 213283 | GCCGGGCGCGGTGGCGCGTGCCTGTAGTCCCAGCTACTCGGGAGGCTGAGG |
| | | TGGGAGGATCGCTTGAGCCCAGGAGTTCTGGGCTGTAGTGCGCTATGCCGA |
| | | TCGGGTGTCCGCACTAAGTTCGGCATCAATATGGTGACCTCCCGGGAGCGG |
| | | GGGACCACCAGGTTGCCTAAGGAGGGGTGAACCGGCCCAGGTCGGAAACGG |
| | | AGCAGGTCAAAACTCCCGTGCTGATCAGTAGTGGGATCGCGCCTGTGAATA |
| | | GCCACTGCACTCCAGCCTGAGCAACATAGCGAGACCCCGTCTCTT |
| RN7SL5P | 213284 | GCCGGGCGCGGTGGCGCGTGCCTGTGGTCCCAGCTACTCGGGAGGCTGAGG |
| | | CTGGAGGATCGCTTGAGTCCAGGAGTTCTGGGCTGTAGTGCGCTATGCCGA |
| | | TCGGGTGTCCGCACTAAGTTCGGCATCAATATGGTGACCTCCCGGGAGCGG |
| | | GGGACCACCAGGTTGCCTAAGGAGGGGTGAACCGGCCCAGGTCGGAAACGG |
| | | AGCAGGTCAAAACTCCCGTGCTGATCAGTAGAAGTCTGTAATGCTACTGGT |
| | | GTCCCCTAATTTTCTTATAGCCACAGTTCCTTTCGCCTGAGCTCATTACAG |
| | | AGACAAATATCCATT |
| RPPH1 | 213285 | GGCGGAGGGAAGCTCATCAGTGGGGCCACGAGCTGAGTGCGTCCTGTCACT |
| | | CCACTCCCATGTCCCTTGGGAAGGTCTGAGACTAGGGCCAGAGGCGGCCCT |
| | | AACAGGGCTCTCCCTGAGCTTCGGGGAGGTGAGTTCCCAGAGAACGGGGCT |
| | | CCGCGCGAGGTCAGACTGGGCAGGAGATGCCGTGGACCCCGCCCTTCGGGG |
| | | AGGGGCCCGGCGGATGCCTCCTTTGCCGGAGCTTGGAACAGACTCACGGCC |
| | | AGCGAAGTGAGTTCAATGGCTGAGGTGAGGTACCCCGCAGGGGACCTCATA |
| | | ACCCAATTCAGACTACTCTCCTCCGCC |
| SNORD3A with | 213286 | AAGACTATACTTTCAGGGATCATTTCTATAGTGTGTTACTAGAGAAGTTTC |
| the ALU region in | | TCTGAACGTGTAGAGCACCGAAAACCACGAGGAAGAGAGGTAGCGTTTTCT |
| bold and italics, in | | CCTGAGCGTGAAGCCGGCTTTCTGGCGTTGCTTGGCTGCAACTGCCGTCAG |
| some embodiments | | CCATTGATGATCGTTCTTCTCTCCGTATTGGGGAGTGAGAGGGAGAGAACG |
| the ALU region | | CGGTCTGAGTGGTTTTTCCTTCTTGATGGCTCAATGACAGAGACTAGCTCG |
| was not used to | | TAAACTCCGGGGCGTTTCTGGGCTGTTCGCTCCTGCTTGGCATGTCGCGAG |
| generate probes | | AAAGGTTTTCGCCTCCTGTTTCAGCGGTGACGGCTCTTGGGTTTTCTCGGG |
| because it is a | | GTGGCTTTTTAATTTTAGTCTTGGCGCGAGGCGGGGGATGCTGTGTGGCAC |
| repetitive region in | | CTCCTATTGTCTCTTTTTGCGTTTTCTCCCATTCTCGCTCCCTCTTTTGTC |
| other areas of the | | GCCGTTTCCCGCCCGCCACTCCCACCCCCAGACGGGGTCTCCGGGTCTCTT |
| genome. | | GTTCTGTCTGCCGGCCCCGGCTGGATTGCAGTGGCGCGATCTCGGCTCCTA |
| | | GCAACATCTGCCTCCCGGGCTCAAGCGAGTCTCCCGCCTAAGCCCTCCCGA |
| | | GTAGCCGGGGCTTAAAGGCGCACACGCCACTCCAGGCTTTTTTTTTTTTTT |
| | | TTTTTTTTTTTTTGGCAGAAACGGGGTGTCAGCATG |
| Reverse | 213287 | AGAAAGGCAGACTGCCACATGCAGCGCCTCATTTGGATGTGTCTGGAGTCT |
| complement of | | TGGAAGCTTGACTACCCTACGTTCTCCTACAAATGGACCTTGAGAGCTTGT |
| RN7SK with probe | | TTGGAGGTTCTAGCAGGGGAGCGCAGCTACTCGTATACCCTTGACCGAAGA |
| sequences in bold | | CCGGTCCTCCTCTATCGGGGATGGTCGTCCTCTTCGACCGAGCGCGCAGCT |
| and italics (and | | TCGGGAGGGACGCACATGGAGCGGTGAGGGAGGAAGGGGACACCCGCCTAG |
| with gaps between | | CCAGCCAGATCAGCCGAATCAACCCTGGCGATCAATGGGGTGACAGATGTC |
| the probes) | | GCAGCCAGATCGCCCTCACATC |
| Probe for RN7SK | 213288 | AGAAAGGCAGACTGCCACATGCAGCGCCTCATTTGGATGTGTCTGGAGTC |
| Probe for RN7SK | 213289 | CCCTACGTTCTCCTACAAATGGACCTTGAGAGCTTGTTTGGAGGTTCTAG |
| Probe for RN7SK | 213290 | ACTCGTATACCCTTGACCGAAGACCGGTCCTCCTCTATCGGGGATGGTCG |
| Probe for RN7SK | 213291 | CGCGCAGCTTCGGGAGGGACGCACATGGAGCGGTGAGGGAGGAAGGGGAC |
| Probe for RN7SK | 213292 | CAGATCAGCCGAATCAACCCTGGCGATCAATGGGGTGACAGATGTCGCAG |
| Probe for RN7SL1 | 213293 | AGAGACGGGGTCTCGCTATGTTGCCCAGGCTGGAGTGCAGTGGCTATTCA |
| Probe for RN7SL1 | 213294 | TACTGATCAGCACGGGAGTTTTGACCTGCTCCGTTTCCGACCTGGGCCGG |
| Probe for RN7SL1 | 213295 | GCAACCTGGTGGTCCCCCGCTCCCGGGAGGTCACCATATTGATGCCGAAC |
| Probe for RN7SL1 | 213296 | GATCGGCATAGCGCACTACAGCCCAGAACTCCTGGACTCAAGCGATCCTC |
| Probe for RN7SL2 | 213297 | AAGAGACGGGGTCTCGCTATGTTGCTCAGGCTGGAGTGCAGTGGCTATTC |
| Probe for RN7SL2 | 213298 | CTACTGATCAGCACGGGAGTTTTGACCTGCTCCGTTTCCGACCTGGGCCG |
| Probe for RN7SL2 | 213299 | GGCAACCTGGTGGTCCCCCGCTCCCGGGAGGTCACCATATTGATGCCGAA |
| Probe for RN7SL2 | 213300 | CGATCGGCATAGCGCACTACAGCCCAGAACTCCTGGGCTCAAGCGATCCT |
| Probe | 213301 | AATGGATATTTGTCTCTGTAATGAGCTCAGGCGAAAGGAACTGTGGCTAT |
| for RN7SL5P | | |
| Probe | 213302 | CACCAGTAGCATTACAGACTTCTACTGATCAGCACGGGAGTTTTGACCTG |
| for RN7SL5P | | |
| Probe | 213303 | GGGCCGGTTCACCCCTCCTTAGGCAACCTGGTGGTCCCCCGCTCCCGGGA |
| for RN7SL5P | | |
| Probe | 213304 | GCCGAACTTAGTGCGGACACCCGATCGGCATAGCGCACTACAGCCCAGAA |
| for RN7SL5P | | |
| Probe | 213305 | GATCCTCCAGCCTCAGCCTCCCGAGTAGCTGGGACCACAGGCACGCGCCA |
| for RN7SL5P | | |
| Probe for RPPH1 | 213306 | GGCGGAGGAGAGTAGTCTGAATTGGGTTATGAGGTCCCCTGCGGGGTACC |
| Probe for RPPH1 | 213307 | AACTCACTTCGCTGGCCGTGAGTCTGTTCCAAGCTCCGGCAAAGGAGGCA |
| Probe for RPPH1 | 213308 | CCCGAAGGGCGGGGTCCACGGCATCTCCTGCCCAGTCTGACCTCGCGCGG |
| Probe for RPPH1 | 213309 | GAACTCACCTCCCCGAAGCTCAGGGAGAGCCCTGTTAGGGCCGCCTCTGG |
| Probe for RPPH1 | 213310 | TTCCCAAGGGACATGGGAGTGGAGTGACAGGACGCACTCAGCTCGTGGCC |
| Probe | 213311 | CCCGGAGACCCCGTCTGGGGGTGGGAGTGGCGGGCGGGAAACGGCGACAA |
| for SNORD3A | | |
| Probe | 213312 | TGGGAGAAAACGCAAAAAGAGACAATAGGAGGTGCCACACAGCATCCCCC |
| for SNORD3A | | |
| Probe | 213313 | TAAAATTAAAAAGCCACCCCGAGAAAACCCAAGAGCCGTCACCGCTGAAA |
| for SNORD3A | | |
| Probe | 213314 | TTTCTCGCGACATGCCAAGCAGGAGCGAACAGCCCAGAAACGCCCCGGAG |
| for SNORD3A | | |
| Probe | 213315 | CTGTCATTGAGCCATCAAGAAGGAAAAACCACTCAGACCGCGTTCTCTCC |
| for SNORD3A | | |
| Probe for | 213316 | ACGGAGAGAAGAACGATCATCAATGGCTGACGGCAGTTGCAGCCAAGCAA |
| SNORD3A | | |
| Probe for | 213317 | TTCACGCTCAGGAGAAAACGCTACCTCTCTTCCTCGTGGTTTTCGGTGCT |
| SNORD3A | | |
| Probe for | 213318 | AAACTTCTCTAGTAACACACTATAGAAATGATCCCTGAAAGTATAGTCTT |
| SNORD3A | | |
| (additional probe | | |
| added at start of | | |
| SNORD3A | | |
| transcript) | | |
| Probe for RN7SL1 | 213319 | CTCAGCCTCCCGAGTAGCTGGGACTACAGGCACGCGCCACCGCGCCCGGC |
| and RN7SL2 | | |
| (additional probe | | |
| added at start of | | |
| RN7SL1 and | | |
| RN7SL2 transcript) | | |

Additional Probes

| | | |
|---|---|---|
| 12S_P1 | 213320 | GTTCGTCCAAGTGCACTTTCCAGTACACTTACCATGTTACGACTTGTCTC |
| 12S_P2 | 213321 | TAGGGGTTTTAGTTAAATGTCCTTTGAAGTATACTTGAGGAGGGTGACGG |
| 12S_P3 | 213322 | TTCAGGGCCCTGTTCAACTAAGCACTCTACTCTCAGTTTACTGCTAAATC |
| 12S_P4 | 213323 | AGTTTCATAAGGGCTATCGTAGTTTTCTGGGGTAGAAAATGTAGCCCATT |
| 12S_P5 | 213324 | GGCTACACCTTGACCTAACGTCTTTACGTGGGTACTTGCGCTTACTTTGT |
| 12S_P6 | 213325 | TTGCTGAAGATGGCGGTATATAGGCTGAGCAAGAGGTGGTGAGGTTGATC |
| 12S_P7 | 213326 | CAGAACAGGCTCCTCTAGAGGGATATGAAGCACCGCCAGGTCCTTTGAGT |
| 12S_P8 | 213327 | GTAGTGTTCTGGCGAGCAGTTTTGTTGATTTAACTGTTGAGGTTTAGGGC |
| 12S_P9 | 213328 | ATCTAATCCCAGTTTGGGTCTTAGCTATTGTGTGTTCAGATATGTTAAAG |
| 12S_P10 | 213329 | ATTTTGTGTCAACTGGAGTTTTTTACAACTCAGGTGAGTTTTAGCTTTAT |
| 12S_P11 | 213330 | CTAAAACACTCTTTACGCCGGCTTCTATTGACTTGGGTTAATCGTGTGAC |
| 12S_P12 | 213331 | GAAATTGACCAACCCTGGGGTTAGTATAGCTTAGTTAAACTTTCGTTTAT |
| 12S_P13 | 213332 | ACTGCTGTTTCCCGTGGGGGTGTGGCTAGGCTAAGCGTTTTGAGCTGCAT |
| 12S_P14 | 213333 | GCTTGTCCCTTTTGATCGTGGTGATTTAGAGGGTGAACTCACTGGAACGG |
| 12S_P15 | 213334 | TAATCTTACTAAGAGCTAATAGAAAGGCTAGGACCAAACCTATTTGTTTA |
| 16S_P1 | 213335 | AAACCCTGTTCTTGGGTGGGTGTGGGTATAATACTAAGTTGAGATGATAT |
| 16S_P2 | 213336 | GCGCTTTGTGAAGTAGGCCTTATTTCTCTTGTCCTTTCGTACAGGGAGGA |
| 16S_P3 | 213337 | AAACCGACCTGGATTACTCCGGTCTGAACTCAGATCACGTAGGACTTTAA |
| 16S_P4 | 213338 | ACCTTTAATAGCGGCTGCACCATCGGGATGTCCTGATCCAACATCGAGGT |
| 16S_P5 | 213339 | TGATATGGACTCTAGAATAGGATTGCGCTGTTATCCCTAGGGTAACTTGT |
| 16S_P6 | 213340 | ATTGGATCAATTGAGTATAGTAGTTCGCTTTGACTGGTGAAGTCTTAGCA |
| 16S_P7 | 213341 | TTGGGTTCTGCTCCGAGGTCGCCCCAACCGAAATTTTTAATGCAGGTTTG |
| 16S_P8 | 213342 | TGGGTTTGTTAGGTACTGTTTGCATTAATAAATTAAAGCTCCATAGGGTC |
| 16S_P9 | 213343 | GTCATGCCCGCCTCTTCACGGGCAGGTCAATTTCACTGGTTAAAAGTAAG |
| 16S_P10 | 213344 | CGTGGAGCCATTCATACAGGTCCCTATTTAAGGAACAAGTGATTATGCTA |
| 16S_P11 | 213345 | GGTACCGCGGCCGTTAAACATGTGTCACTGGGCAGGCGGTGCCTCTAATA |
| 16S_P12 | 213346 | GTGATGTTTTTGGTAAACAGGCGGGGTAAGGTTTGCCGAGTTCCTTTTAC |
| 16S_P13 | 213347 | CTTATGAGCATGCCTGTGTTGGGTTGACAGTGAGGGTAATAATGACTTGT |
| 16S_P14 | 213348 | ATTGGGCTGTTAATTGTCAGTTCAGTGTTTTGATCTGACGCAGGCTTATG |
| 16S_P15 | 213349 | TCATGTTACTTATACTAACATTAGTTCTTCTATAGGGTGATAGATTGGTC |
| 16S_P16 | 213350 | AGTTCAGTTATATGTTTGGGATTTTTTAGGTAGTGGGTGTTGAGCTTGAA |
| 16S_P17 | 213351 | TGGCTGCTTTTAGGCCTACTATGGGTGTTAAATTTTTTACTCTCTCTACA |
| 16S_P18 | 213352 | GTCCAAAGAGCTGTTCCTCTTTGGACTAACAGTTAAATTTACAAGGGGAT |
| 16S_P19 | 213353 | GGCAAATTTAAAGTTGAACTAAGATTCTATCTTGGACAACCAGCTATCAC |
| 16S_P20 | 213354 | TGTCGCCTCTACCTATAAATCTTCCCACTATTTTGCTACATAGACGGGTG |
| 16S_P21 | 213355 | TCTTAGGTAGCTCGTCTGGTTTCGGGGGTCTTAGCTTTGGCTCTCCTTGC |
| 16S_P22 | 213356 | TAATTCATTATGCAGAAGGTATAGGGGTTAGTCCTTGCTATATTATGCTT |
| 16S_P23 | 213357 | TCTTTCCCTTGCGGTACTATATCTATTGCGCCAGGTTTCAATTTCTATCG |
| 16S_P24 | 213358 | GGTAAATGGTTTGGCTAAGGTTGTCTGGTAGTAAGGTGGAGTGGGTTTGG |
| 18S_P1 | 213359 | TAATGATCCTTCCGCAGGTTCACCTACGGAAACCTTGTTACGACTTTTAC |
| 18S_P2 | 213360 | AAGTTCGACCGTCTTCTCAGCGCTCCGCCAGGGCCGTGGGCCGACCCCGG |
| 18S_P3 | 213361 | GGCCTCACTAAACCATCCAATCGGTAGTAGCGACGGGCGGTGTGTACAAA |
| 18S_P4 | 213362 | CAACGCAAGCTTATGACCCGCACTTACTCGGGAATTCCCTCGTTCATGGG |
| 18S_P5 | 213363 | CCGATCCCCATCACGAATGGGGTTCAACGGGTTACCCGCGCCTGCCGGCG |
| 18S_P6 | 213364 | CTGAGCCAGTCAGTGTAGCGCGCGTGCAGCCCCGGACATCTAAGGGCATC |
| 18S_P7 | 213365 | CTCAATCTCGGGTGGCTGAACGCCACTTGTCCCTCTAAGAAGTTGGGGGA |
| 18S_P8 | 213366 | GGTCGCGTAACTAGTTAGCATGCCAGAGTCTCGTTCGTTATCGGAATTAA |
| 18S_P9 | 213367 | CACCAACTAAGAACGGCCATGCACCACCACCCACGGAATCGAGAAAGAGC |
| 18S_P10 | 213368 | CCTGTCCGTGTCCGGGCCGGGTGAGGTTTCCCGTGTTGAGTCAAATTAAG |
| 18S_P11 | 213369 | CTGGTGGTGCCCTTCCGTCAATTCCTTTAAGTTTCAGCTTTGCAACCATA |
| 18S_P12 | 213370 | AAAGACTTTGGTTTCCCGGAAGCTGCCCGGCGGGTCATGGGAATAACGCC |
| 18S_P13 | 213371 | GGCATCGTTTATGGTCGGAACTACGACGGTATCTGATCGTCTTCGAACCT |
| 18S_P14 | 213372 | GATTAATGAAAACATTCTTGGCAAATGCTTTCGCTCTGGTCCGTCTTGCG |
| 18S_P15 | 213373 | CACCTCTAGCGGCGCAATACGAATGCCCCCGGCCGTCCCTCTTAATCATG |
| 18S_P16 | 213374 | ACCAACAAAATAGAACCGCGGTCCTATTCCATTATTCCTAGCTGCGGTAT |
| 18S_P17 | 213375 | CTGCTTTGAACACTCTAATTTTTTCAAAGTAAACGCTTCGGGCCCCGCGG |
| 18S_P18 | 213376 | GCATCGAGGGGGCGCCGAGAGGCAAGGGGCGGGGACGGGCGGTGGCTCGC |
| 18S_P19 | 213377 | CCGCCCGCTCCCAAGATCCAACTACGAGCTTTTTAACTGCAGCAACTTTA |
| 18S_P20 | 213378 | GCTGGAATTACCGCGGCTGCTGGCACCAGACTTGCCCTCCAATGGATCCT |
| 18S_P21 | 213379 | AGTGGACTCATTCCAATTACAGGGCCTCGAAAGAGTCCTGTATTGTTATT |
| 18S_P22 | 213380 | CCCGGGTCGGGAGTGGGTAATTTGCGCGCCTGCTGCCTTCCTTGGATGTG |
| 18S_P23 | 213381 | GCTCCCTCTCCGGAATCGAACCCTGATTCCCCGTCACCCGTGGTCACCAT |
| 18S_P24 | 213382 | TACCATCGAAAGTTGATAGGGCAGACGTTCGAATGGGTCGTCGCCGCCAC |
| 18S_P25 | 213383 | GGCCCGAGGTTATCTAGAGTCACCAAAGCCGCCGGCGCCCGCCCCCCGGC |
| 18S_P26 | 213384 | GCTGACCGGGTTGGTTTTGATCTGATAAATGCACGCATCCCCCCCGCGAA |
| 18S_P27 | 213385 | TCGGCATGTATTAGCTCTAGAATTACCACAGTTATCCAAGTAGGAGAGGA |
| 18S_P28 | 213386 | AACCATAACTGATTTAATGAGCCATTCGCAGTTTCACTGTACCGGCCGTG |
| 18S_P29 | 213387 | ATGGCTTAATCTTTGAGACAAGCATATGCTACTGGCAGGATCAACCAGGT |
| 28S_P1 | 213388 | GACAAACCCTTGTGTCGAGGGCTGACTTTCAATAGATCGCAGCGAGGGAG |
| 28S_P2 | 213389 | CGAAACCCCGACCCAGAAGCAGGTCGTCTACGAATGGTTTAGCGCCAGGT |
| 28S_P3 | 213390 | GGTGCGTGACGGGCGAGGGGGCGGCCGCCTTTCCGGCCGCGCCCCGTTTC |
| 28S_P4 | 213391 | CTCCGCACCGGACCCCGGTCCCGGCGCGCGGCGGGGCACGCGCCCTCCCG |
| 28S_P5 | 213392 | AGGGGGGGGCGGCCCGCCGGCGGGGACAGGCGGGGGACCGGCTATCCGAG |
| 28S_P6 | 213393 | GCGGCGCTGCCGTATCGTTCGCCTGGGCGGGATTCTGACTTAGAGGCGTT |
| 28S_P7 | 213394 | AGATGGTAGCTTCGCCCCATTGGCTCCTCAGCCAAGCACATACACCAAAT |
| 28S_P8 | 213395 | TCCTCTCGTACTGAGCAGGATTACCATGGCAACAACACATCATCAGTAGG |
| 28S_P9 | 213396 | CTCACGACGGTCTAAACCCAGCTCACGTTCCCTATTAGTGGGTGAACAAT |
| 28S_P10 | 213397 | TTCTGCTTCACAATGATAGGAAGAGCCGACATCGAAGGATCAAAAAGCGA |
| 28S_P11 | 213398 | TTGGCCGCCACAAGCCAGTTATCCCTGTGGTAACTTTTCTGACACCTCCT |
| 28S_P12 | 213399 | GGTCAGAAGGATCGTGAGGCCCCGCTTTCACGGTCTGTATTCGTACTGAA |
| 28S_P13 | 213400 | AGCTTTTGCCCTTCTGCTCCACGGGAGGTTTCTGTCCTCCCTGAGCTCGC |
| 28S_P14 | 213401 | TTACCGTTTGACAGGTGTACCGCCCCAGTCAAACTCCCCACCTGGCACTG |
| 28S_P15 | 213402 | GCGCCCGGCCGGGCGGGCGCTTGGCGCCAGAAGCGAGAGCCCCTCGGGCT |
| 28S_P16 | 213403 | CCGGGTCAGTGAAAAAACGATCAGAGTAGTGGTATTTCACCGGCGGCCCG |
| 28S_P17 | 213404 | CGCCCCGGGCCCCTCGCGGGGACACCGGGGGGGCGCCGGGGGCCTCCCAC |
| 28S_P18 | 213405 | CATGTCTCTTCACCGTGCCAGACTAGAGTCAAGCTCAACAGGGTCTTCTT |
| 28S_P19 | 213406 | CCAAGCCCGTTCCCTTGGCTGTGGTTTCGCTGGATAGTAGGTAGGGACAG |
| 28S_P20 | 213407 | TCCATTCATGCGCGTCACTAATTAGATGACGAGGCATTTGGCTACCTTAA |
| 28S_P21 | 213408 | TCCCGCCGTTTACCCGCGCTTCATTGAATTTCTTCACTTTGACATTCAGA |
| 28S_P22 | 213409 | CACATCGCGTCAACACCCGCCGCGGGCCTTCGCGATGCTTTGTTTTAATT |
| 28S_P23 | 213410 | CCTGGTCCGCACCAGTTCTAAGTCGGCTGCTAGGCGCCGGCCGAGGCGAG |
| 28S_P24 | 213411 | CGGCCCCGGGGGGGGACCCGGCGGGGGGGACCGGCCCGCGGCCCCTCCGC |
| 28S_P25 | 213412 | CCGCCGCGCGCCGAGGAGGAGGGGGGAACGGGGGGCGGACGGGGCCGGGG |
| 28S_P26 | 213413 | ACGAACCGCCCCGCCCCGCCGCCCGCCGACCGCCGCCGCCCGACCGCTCC |
| 28S_P27 | 213414 | CGCGCGCGACCGAGACGTGGGGTGGGGGTGGGGGGCGCGCCGCGCCGCCG |
| 28S_P28 | 213415 | GCGGCCGCGACGCCCGCCGCAGCTGGGGCGATCCACGGGAAGGGCCCGGC |
| 28S_P29 | 213416 | GCGCCGCCGCCGGCCCCCCGGGTCCCCGGGGCCCCCCTCGCGGGGACCTG |
| 28S_P30 | 213417 | CCGGCGGCCGCCGCGCGGCCCCTGCCGCCCCGACCCTTCTCCCCCCGCCG |
| 28S_P31 | 213418 | CTCCCCCGGGGAGGGGGGAGGACGGGGAGCGGGGGAGAGAGAGAGAGAGA |
| 28S_P32 | 213419 | AGGGAGCGAGCGGCGCGCGCGGGTGGGGCGGGGGAGGGCCGCGAGGGGGG |
| 28S_P33 | 213420 | GGGGGCGCGCGCCTCGTCCAGCCGCGGCGCGCGCCCAGCCCCGCTTCGCG |
| 28S_P34 | 213421 | CCCAGCCCTTAGAGCCAATCCTTATCCCGAAGTTACGGATCCGGCTTGCC |
| 28S_P35 | 213422 | CATTGTTCCAACATGCCAGAGGCTGTTCACCTTGGAGACCTGCTGCGGAT |
| 28S_P36 | 213423 | CGCGAGATTTACACCCTCTCCCCCGGATTTTCAAGGGCCAGCGAGAGCTC |
| 28S_P37 | 213424 | AACCGCGACGCTTTCCAAGGCACGGGCCCCTCTCTCGGGGCGAACCCATT |
| 28S_P38 | 213425 | CTTCACAAAGAAAAGAGAACTCTCCCCGGGGCTCCCGCCGGCTTCTCCGG |
| 28S_P39 | 213426 | CGCACTGGACGCCTCGCGGCGCCCATCTCCGCCACTCCGGATTCGGGGAT |
| 28S_P40 | 213427 | TTTCGATCGGCCGAGGGCAACGGAGGCCATCGCCCGTCCCTTCGGAACGG |
| 28S_P41 | 213428 | CAGGACCGACTGACCCATGTTCAACTGCTGTTCACATGGAACCCTTCTCC |
| 28S_P42 | 213429 | GTTCTCGTTTGAATATTTGCTACTACCACCAAGATCTGCACCTGCGGCGG |
| 28S_P43 | 213430 | CGCCCTAGGCTTCAAGGCTCACCGCAGCGGCCCTCCTACTCGTCGCGGCG |
| 28S_P44 | 213431 | TCCGGGGGCGGGGAGCGGGGCGTGGGCGGGAGGAGGGGAGGAGGCGTGGG |
| 28S_P45 | 213432 | AGGACCCCACACCCCCGCCGCCGCCGCCGCCGCCGCCCTCCGACGCACAC |
| 28S_P46 | 213433 | GCGCGCCGCCCCCGCCGCTCCCGTCCACTCTCGACTGCCGGCGACGGCCG |
| 28S_P47 | 213434 | CTCCAGCGCCATCCATTTTCAGGGCTAGTTGATTCGGCAGGTGAGTTGTT |
| 28S_P48 | 213435 | GATTCCGACTTCCATGGCCACCGTCCTGCTGTCTATATCAACCAACACCT |
| 28S_P49 | 213436 | GAGCGTCGGCATCGGGCGCCTTAACCCGGCGTTCGGTTCATCCCGCAGCG |
| 28S_P50 | 213437 | AAAAGTGGCCCACTAGGCACTCGCATTCCACGCCCGGCTCCACGCCAGCG |
| 28S_P51 | 213438 | CCATTTAAAGTTTGAGAATAGGTTGAGATCGTTTCGGCCCCAAGACCTCT |
| 28S_P52 | 213439 | CGGATAAAACTGCGTGGCGGGGGTGCGTCGGGTCTGCGAGAGCGCCAGCT |
| 28S_P53 | 213440 | TCGGAGGGAACCAGCTACTAGATGGTTCGATTAGTCTTTCGCCCCTATAC |
| 28S_P54 | 213441 | GATTTGCACGTCAGGACCGCTACGGACCTCCACCAGAGTTTCCTCTGGCT |
| 28S_P55 | 213442 | ATAGTTCACCATCTTTCGGGTCCTAACACGTGCGCTCGTGCTCCACCTCC |
| 28S_P56 | 213443 | AGACGGGCCGGTGGTGCGCCCTCGGCGGACTGGAGAGGCCTCGGGATCCC |
| 28S_P57 | 213444 | CGCGCCGGCCTTCACCTTCATTGCGCCACGGCGGCTTTCGTGCGAGCCCC |
| 28S_P58 | 213445 | TTAGACTCCTTGGTCCGTGTTTCAAGACGGGTCGGGTGGGTAGCCGACGT |
| 28S_P59 | 213446 | GCGCTCGCTCCGCCGTCCCCCTCTTCGGGGGACGCGCGCGTGGCCCCGAG |
| 28S_P60 | 213447 | CCCGACGGCGCGACCCGCCCGGGGCGCACTGGGGACAGTCCGCCCCGCCC |
| 28S_P61 | 213448 | GCACCCCCCCCGTCGCCGGGGCGGGGGCGCGGGGAGGAGGGGTGGGAGAG |
| 28S_P62 | 213449 | AGGGGTGGCCCGGCCCCCCCACGAGGAGACGCCGGCGCGCCCCCGCGGGG |
| 28S_P63 | 213450 | GGGGATTCCCCGCGGGGGTGGGCGCCGGGAGGGGGGAGAGCGCGGCGACG |
| 28S_P64 | 213451 | GCCCCGGGATTCGGCGAGTGCTGCTGCCGGGGGGGCTGTAACACTCGGGG |
| 28S_P65 | 213452 | CCGCCCCCGCCGCCGCCGCCACCGCCGCCGCCGCCGCCGCCCCGACCCGC |
| 28S_P66 | 213453 | AGGACGCGGGGCCGGGGGGCGGAGACGGGGGAGGAGGAGGACGGACGGAC |
| 28S_P67 | 213454 | AGCCACCTTCCCCGCCGGGCCTTCCCAGCCGTCCCGGAGCCGGTCGCGGC |
| 28S_P68 | 213455 | AAATGCGCCCGGCGGCGGCCGGTCGCCGGTCGGGGGACGGTCCCCCGCCG |
| 28S_P69 | 213456 | CCGCCCGCCCACCCCCGCACCCGCCGGAGCCCGCCCCCTCCGGGGAGGAG |
| 28S_P70 | 213457 | GGGAAGGGAGGGCGGGTGGAGGGGTCGGGAGGAACGGGGGGCGGGAAAGA |
| 28S_P71 | 213458 | ACACGGCCGGACCCGCCGCCGGGTTGAATCCTCCGGGCGGACTGCGCGGA |
| 28S_P72 | 213459 | TCTTAACGGTTTCACGCCCTCTTGAACTCTCTCTTCAAAGTTCTTTTCAA |
| 28S_P73 | 213460 | CTTGTTGACTATCGGTCTCGTGCCGGTATTTAGCCTTAGATGGAGTTTAC |
| 28S_P74 | 213461 | GCATTCCCAAGCAACCCGACTCCGGGAAGACCCGGGCGCGCGCCGGCCGC |
| 28S_P75 | 213462 | GTCCACGGGCTGGGCCTCGATCAGAAGGACTTGGGCCCCCCACGAGCGGC |
| 28S_P76 | 213463 | TTCCGTACGCCACATGTCCCGCGCCCCGCGGGGCGGGGATTCGGCGCTGG |
| 28S_P77 | 213464 | CTCGCCGTTACTGAGGGAATCCTGGTTAGTTTCTTTTCCTCCGCTGACTA |
| 28S_P78 | 213465 | GCGGGTCGCCACGTCTGATCTGAGGTCGCGTCTCGGAGGGGGACGGGCCG |
| 5.8S_P1 | 213466 | AAGCGACGCTCAGACAGGCGTAGCCCCGGGAGGAACCCGGGGCCGCAAGT |
| 5.8S_P3 | 213467 | GCAGCTAGCTGCGTTCTTCATCGACGCACGAGCCGAGTGATCCACCGCTA |
| 5S_P1 | 213468 | AAAGCCTACAGCACCCGGTATTCCCAGGCGGTCTCCCATCCAAGTACTAA |
| 5S_P3 | 213469 | TTCCGAGATCAGACGAGATCGGGCGCGTTCAGGGTGGTATGGCCGTAGAC |
| HBA1_P1 | 213470 | GCCGCCCACTCAGACTTTATTCAAAGACCACGGGGGTACGGGTGCAGGAA |
| HBA1_P2 | 213471 | GGGGGAGGCCCAAGGGGCAAGAAGCATGGCCACCGAGGCTCCAGCTTAAC |
| HBA1_P3 | 213472 | GCACGGTGCTCACAGAAGCCAGGAACTTGTCCAGGGAGGCGTGCACCGCA |
| HBA1_P4 | 213473 | GGGAGGTGGGCGGCCAGGGTCACCAGCAGGCAGTGGCTTAGGAGCTTGAA |
| HBA1_P5 | 213474 | CCGAAGCTTGTGCGCGTGCAGGTCGCTCAGGGCGGACAGCGCGTTGGGCA |
| HBA1_P6 | 213475 | CCACGGCGTTGGTCAGCGCGTCGGCCACCTTCTTGCCGTGGCCCTTAACC |
| HBA1_P7 | 213476 | CTCAGGTCGAAGTGCGGGAAGTAGGTCTTGGTGGTGGGGAAGGACAGGAA |
| HBA1_P8 | 213477 | CTCCGCACCATACTCGCCAGCGTGCGCGCCGACCTTACCCCAGGCGGCCT |
| HBA1_P9 | 213478 | CGGCAGGAGACAGCACCATGGTGGGTTCTCTCTGAGTCTGTGGGGACCAG |
| HBA2_P1 | 213479 | GAGGGGAGGAGGGCCCGTTGGGAGGCCCAGCGGGCAGGAGGAACGGCTAC |
| HBA2_P2 | 213480 | ACGGTATTTGGAGGTCAGCACGGTGCTCACAGAAGCCAGGAACTTGTCCA |
| HBA2_P3 | 213481 | CAGGGGTGAACTCGGCGGGGAGGTGGGCGGCCAGGGTCACCAGCAGGCAG |
| HBA2_P4 | 213482 | AAGTTGACCGGGTCCACCCGAAGCTTGTGCGCGTGCAGGTCGCTCAGGGC |
| HBA2_P5 | 213483 | CATGTCGTCCACGTGCGCCACGGCGTTGGTCAGCGCGTCGGCCACCTTCT |
| HBA2_P6 | 213484 | CCTGGGCAGAGCCGTGGCTCAGGTCGAAGTGCGGGAAGTAGGTCTTGGTG |
| HBA2_P7 | 213485 | AACATCCTCTCCAGGGCCTCCGCACCATACTCGCCAGCGTGCGCGCCGAC |
| HBA2_P8 | 213486 | CTTGACGTTGGTCTTGTCGGCAGGAGACAGCACCATGGTGGGTTCTCTCT |
| HBB_P1 | 213487 | GCAATGAAAATAAATGTTTTTTATTAGGCAGAATCCAGATGCTCAAGGCC |
| HBB_P2 | 213488 | CAGTTTAGTAGTTGGACTTAGGGAACAAAGGAACCTTTAATAGAAATTGG |
| HBB_P3 | 213489 | GCTTAGTGATACTTGTGGGCCAGGGCATTAGCCACACCAGCCACCACTTT |
| HBB_P4 | 213490 | CACTGGTGGGGTGAATTCTTTGCCAAAGTGATGGGCCAGCACACAGACCA |
| HBB_P5 | 213491 | GCCTGAAGTTCTCAGGATCCACGTGCAGCTTGTCACAGTGCAGCTCACTC |
| HBB_P6 | 213492 | CCCTTGAGGTTGTCCAGGTGAGCCAGGCCATCACTAAAGGCACCGAGCAC |
| HBB_P7 | 213493 | CTTCACCTTAGGGTTGCCCATAACAGCATCAGGAGTGGACAGATCCCCAA |
| HBB_P8 | 213494 | TCTGGGTCCAAGGGTAGACCACCAGCAGCCTGCCCAGGGCCTCACCACCA |
| HBB_P9 | 213495 | ACCTTGCCCCACAGGGCAGTAACGGCAGACTTCTCCTCAGGAGTCAGATG |
| HBG1_P1 | 213496 | GTGATCTCTCAGCAGAATAGATTTATTATTTGTATTGCTTGCAGAATAAA |
| HBG1_P2 | 213497 | CTCTGAATCATGGGCAGTGAGCTCAGTGGTATCTGGAGGACAGGGCACTG |
| HBG1_P3 | 213498 | ATCTTCTGCCAGGAAGCCTGCACCTCAGGGGTGAATTCTTTGCCGAAATG |
| HBG1_P4 | 213499 | CACCAGCACATTTCCCAGGAGCTTGAAGTTCTCAGGATCCACATGCAGCT |
| HBG1_P5 | 213500 | CACTCAGCTGGGCAAAGGTGCCCTTGAGATCATCCAGGTGCTTTGTGGCA |
| HBG1_P6 | 213501 | AGCACCTTCTTGCCATGTGCCTTGACTTTGGGGTTGCCCATGATGGCAGA |
| HBG1_P7 | 213502 | GCCAAAGCTGTCAAAGAACCTCTGGGTCCATGGGTAGACAACCAGGAGCC |
| HBG1_P8 | 213503 | CTCCAGCATCTTCCACATTCACCTTGCCCCACAGGCTTGTGATAGTAGCC |
| HBG1_P9 | 213504 | AAATGACCCATGGCGTCTGGACTAGGAGCTTATTGATAACCTCAGACGTT |
| HBG2_P1 | 213505 | GTGATCTCTTAGCAGAATAGATTTATTATTTGATTGCTTGCAGAATAAAG |
| HBG2_P2 | 213506 | TCTGCATCATGGGCAGTGAGCTCAGTGGTATCTGGAGGACAGGGCACTGG |
| HBG2_P3 | 213507 | TCTTCTGCCAGGAAGCCTGCACCTCAGGGGTGAATTCTTTGCCGAAATGG |
| HBG2_P4 | 213508 | ACCAGCACATTTCCCAGGAGCTTGAAGTTCTCAGGATCCACATGCAGCTT |
| HBG2_P5 | 213509 | ACTCAGCTGGGCAAAGGTGCCCTTGAGATCATCCAGGTGCTTTATGGCAT |
| HBG2_P6 | 213510 | GCACCTTCTTGCCATGTGCCTTGACTTTGGGGTTGCCCATGATGGCAGAG |
| HBG2_P7 | 213511 | CCAAAGCTGTCAAAGAACCTCTGGGTCCATGGGTAGACAACCAGGAGCCT |
| HBG2_P8 | 213512 | TCCAGCATCTTCCACATTCACCTTGCCCCACAGGCTTGTGATAGTAGCCT |
| HBG2_P9 | 213513 | AATGACCCATGGCGTCTGGACTAGGAGCTTATTGATAACCTCAGACGTTC |
| 5S_GNbac_P1 | 213514 | ATGCCTGGCAGTTCCCTACTCTCGCATGGGGAGACCCCACACTACCATCG |
| 5S_GNbac_P2 | 213515 | ACTTCTGAGTTCGGCATGGGGTCAGGTGGGACCACCGCGCTACGGCCGCC |
| 16S_GNbac_P1 | 213516 | GGTTACCTTGTTACGACTTCACCCCAGTCATGAATCACAAAGTGGTAAGT |
| 16S_GNbac_P2 | 213517 | AAGCTACCTACTTCTTTTGCAACCCACTCCCATGGTGTGACGGGCGGTGT |
| 16S_GNbac_P3 | 213518 | ACGTATTCACCGTGGCATTCTGATCCACGATTACTAGCGATTCCGACTTC |
| 16S_GNbac_P4 | 213519 | AGACTCCAATCCGGACTACGACGCACTTTATGAGGTCCGCTTGCTCTCGC |
| 16S_GNbac_P5 | 213520 | TGTATGCGCCATTGTAGCACGTGTGTAGCCCTGGTCGTAAGGGCCATGAT |
| 16S_GNbac_P6 | 213521 | CCACCTTCCTCCAGTTTATCACTGGCAGTCTCCTTTGAGTTCCCGGCCGG |
| 16S_GNbac_P7 | 213522 | GGATAAGGGTTGCGCTCGTTGCGGGACTTAACCCAACATTTCACAACACG |
| 16S_GNbac_P8 | 213523 | TGCAGCACCTGTCTCACGGTTCCCGAAGGCACATTCTCATCTCTGAAAAC |
| 16S_GNbac_P9 | 213524 | GACCAGGTAAGGTTCTTCGCGTTGCATCGAATTAAACCACATGCTCCACC |
| 16S_GNbac_P10 | 213525 | CGTCAATTCATTTGAGTTTTAACCTTGCGGCCGTACTCCCCAGGCGGTCG |
| 16S_GNbac_P11 | 213526 | TCCGGAAGCCACGCCTCAAGGGCACAACCTCCAAGTCGACATCGTTTACG |
| 16S_GNbac_P12 | 213527 | GTATCTAATCCTGTTTGCTCCCCACGCTTTCGCACTGAGCGTCAGTCTTC |
| 16S_GNbac_P13 | 213528 | TTCGCCACCGGTATTCCTCCAGATCTCTACGCATTTCACCGCTACACCTG |
| 16S_GNbac_P14 | 213529 | CTACGAGACTCAAGCTTGCCAGTATCAGATGCAGTTCCCAGGTTGAGCCC |
| 16S_GNbac_P15 | 213530 | GACTTAACAAACCGCCTGCGTGCGCTTTACGCCCAGTAATTCCGATTAAC |
| 16S_GNbac_P16 | 213531 | ATTACCGCGGCTGCTGGCACGGAGTTAGCCGGTGCTTCTTCTGCGGGTAA |
| 16S_GNbac_P17 | 213532 | GTATTAACTTTACTCCCTTCCTCCCCGCTGAAAGTACTTTACAACCCGAA |
| 16S_GNbac_P18 | 213533 | CGCGGCATGGCTGCATCAGGCTTGCGCCCATTGTGCAGTATTCCCCACTG |
| 16S_GNbac_P19 | 213534 | GTCTGGACCGTGTCTCAGTTCCAGTGTGGCTGGTCATCCTCTCAGACCAG |
| 16S_GNbac_P20 | 213535 | TAGGTGAGCCGTTACCCCACCTACTAGCTAATCCCATCTGGGCACATCCG |
| 16S_GNbac_P21 | 213536 | AAGGTCCCCCTCTTTGGTCTTGCGACGTTATGCGGTATTAGCTACCGTTT |
| 16S_GNbac_P22 | 213537 | CTCCATCAGGCAGTTTCCCAGACATTACTCACCCGTCCGCCACTCGTCAG |
| 23S_GNbac_P1 | 213538 | AAGGTTAAGCCTCACGGTTCATTAGTACCGGTTAGCTCAACGCATCGCTG |
| 23S_GNbac_P2 | 213539 | CCTATCAACGTCGTCGTCTTCAACGTTCCTTCAGGACCCTTAAAGGGTCA |
| 23S_GNbac_P3 | 213540 | GGGGCAAGTTTCGTGCTTAGATGCTTTCAGCACTTATCTCTTCCGCATTT |
| 23S_GNbac_P4 | 213541 | CCATTGGCATGACAACCCGAACACCAGTGATGCGTCCACTCCGGTCCTCT |
| 23S_GNbac_P5 | 213542 | CCCCCTCAGTTCTCCAGCGCCCACGGCAGATAGGGACCGAACTGTCTCAC |
| 23S_GNbac_P6 | 213543 | GCTCGCGTACCACTTTAAATGGCGAACAGCCATACCCTTGGGACCTACTT |
| 23S_GNbac_P7 | 213544 | ATGAGCCGACATCGAGGTGCCAAACACCGCCGTCGATATGAACTCTTGGG |
| 23S_GNbac_P8 | 213545 | ATCCCCGGAGTACCTTTTATCCGTTGAGCGATGGCCCTTCCATTCAGAAC |
| 23S_GNbac_P9 | 213546 | ACCTGCTTTCGCACCTGCTCGCGCCGTCACGCTCGCAGTCAAGCTGGCTT |
| 23S_GNbac_P10 | 213547 | CCTCCTGATGTCCGACCAGGATTAGCCAACCTTCGTGCTCCTCCGTTACT |
| 23S_GNbac_P11 | 213548 | GCCCCAGTCAAACTACCCACCAGACACTGTCCGCAACCCGGATTACGGGT |
| 23S_GNbac_P12 | 213549 | AAACATTAAAGGGTGGTATTTCAAGGTCGGCTCCATGCAGACTGGCGTCC |
| 23S_GNbac_P13 | 213550 | CCACCTATCCTACACATCAAGGCTCAATGTTCAGTGTCAAGCTATAGTAA |
| 23S_GNbac_P14 | 213551 | TTCCGTCTTGCCGCGGGTACACTGCATCTTCACAGCGAGTTCAATTTCAC |
| 23S_GNbac_P15 | 213552 | GACAGCCTGGCCATCATTACGCCATTCGTGCAGGTCGGAACTTACCCGAC |
| 23S_GNbac_P16 | 213553 | CTTAGGACCGTTATAGTTACGGCCGCCGTTTACCGGGGCTTCGATCAAGA |
| 23S_GNbac_P17 | 213554 | ACCCCATCAATTAACCTTCCGGCACCGGGCAGGCGTCACACCGTATACGT |
| 23S_GNbac_P18 | 213555 | CACAGTGCTGTGTTTTTAATAAACAGTTGCAGCCAGCTGGTATCTTCGAC |
| 23S_GNbac_P19 | 213556 | CCGCGAGGGACCTCACCTACATATCAGCGTGCCTTCTCCCGAAGTTACGG |
| 23S_GNbac_P20 | 213557 | TTCCTTCACCCGAGTTCTCTCAAGCGCCTTGGTATTCTCTACCTGACCAC |
| 23S_GNbac_P21 | 213558 | GTACGATTTGATGTTACCTGATGCTTAGAGGCTTTTCCTGGAAGCAGGGC |
| 23S_GNbac_P22 | 213559 | ACCGTAGTGCCTCGTCATCACGCCTCAGCCTTGATTTTCCGGATTTGCCT |
| 23S_GNbac_P23 | 213560 | ACGCTTAAACCGGGACAACCGTCGCCCGGCCAACATAGCCTTCTCCGTCC |
| 23S_GNbac_P24 | 213561 | ACCAAGTACAGGAATATTAACCTGTTTCCCATCGACTACGCCTTTCGGCC |
| 23S_GNbac_P25 | 213562 | ACTCACCCTGCCCCGATTAACGTTGGACAGGAACCCTTGGTCTTCCGGCG |
| 23S_GNbac_P26 | 213563 | CGCTTTATCGTTACTTATGTCAGCATTCGCACTTCTGATACCTCCAGCAT |
| 23S_GNbac_P27 | 213564 | TTCGCAGGCTTACAGAACGCTCCCCTACCCAACAACGCATAAGCGTCGCT |
| 23S_GNbac_P28 | 213565 | CATGGTTTAGCCCCGTTACATCTTCCGCGCAGGCCGACTCGACCAGTGAG |
| 23S_GNbac_P29 | 213566 | TAAATGATGGCTGCTTCTAAGCCAACATCCTGGCTGTCTGGGCCTTCCCA |
| 23S_GNbac_P30 | 213567 | AACCATGACTTTGGGACCTTAGCTGGCGGTCTGGGTTGTTTCCCTCTTCA |
| 23S_GNbac_P31 | 213568 | CCCGCCGTGTGTCTCCCGTGATAACATTCTCCGGTATTCGCAGTTTGCAT |
| 23S_GNbac_P32 | 213569 | GGATGACCCCCTTGCCGAAACAGTGCTCTACCCCCGGAGATGAATTCACG |
| 23S_GNbac_P33 | 213570 | AGCTTTCGGGGAGAACCAGCTATCTCCCGGTTTGATTGGCCTTTCACCCC |
| 23S_GNbac_P34 | 213571 | CGCTAATTTTTCAACATTAGTCGGTTCGGTCCTCCAGTTAGTGTTACCCA |
| 23S_GNbac_P35 | 213572 | ATGGCTAGATCACCGGGTTTCGGGTCTATACCCTGCAACTTAACGCCCAG |
| 23S_GNbac_P36 | 213573 | CCTTCGGCTCCCCTATTCGGTTAACCTTGCTACAGAATATAAGTCGCTGA |
| 23S_GNbac_P37 | 213574 | GTACGCAGTCACACGCCTAAGCGTGCTCCCACTGCTTGTACGTACACGGT |
| 23S_GNbac_P38 | 213575 | ACTCCCCTCGCCGGGGTTCTTTTCGCCTTTCCCTCACGGTACTGGTTCAC |
| 23S_GNbac_P39 | 213576 | AGTATTTAGCCTTGGAGGATGGTCCCCCCATATTCAGACAGGATACCACG |
| 23S_GNbac_P40 | 213577 | ATCGAGCTCACAGCATGTGCATTTTTGTGTACGGGGCTGTCACCCTGTAT |
| 23S_GNbac_P41 | 213578 | ACGCTTCCACTAACACACACACTGATTCAGGCTCTGGGCTGCTCCCCGTT |
| 23S_GNbac_P42 | 213579 | GGGGAATCTCGGTTGATTTCTTTTCCTCGGGGTACTTAGATGTTTCAGTT |
| 23S_GNbac_P43 | 213580 | ATTAACCTATGGATTCAGTTAATGATAGTGTGTCGAAACACACTGGGTTT |
| 23S_GNbac_P44 | 213581 | GCCGGTTATAACGGTTCATATCACCTTACCGACGCTTATCGCAGATTAGC |
| 5S_GPbac_P1 | 213582 | GCTTGGCGGCGTCCTACTCTCACAGGGGGAAACCCCCGACTACCATCGGC |
| 5S_GPbac_P2 | 213583 | TTCCGTGTTCGGTATGGGAACGGGTGTGACCTCTTCGCTATCGCCACCAA |
| 16S_GPbac_P1 | 213584 | TAGAAAGGAGGTGATCCAGCCGCACCTTCCGATACGGCTACCTTGTTACG |
| 16S_GPbac_P2 | 213585 | TCTGTCCCACCTTCGGCGGCTGGCTCCTAAAAGGTTACCTCACCGACTTC |
| 16S_GPbac_P3 | 213586 | TCGTGGTGTGACGGGCGGTGTGTACAAGGCCCGGGAACGTATTCACCGCG |
| 16S_GPbac_P4 | 213587 | ATTACTAGCGATTCCAGCTTCACGCAGTCGAGTTGCAGACTGCGATCCGA |
| 16S_GPbac_P5 | 213588 | GTGGGATTGGCTTAACCTCGCGGTTTCGCTGCCCTTTGTTCTGTCCATTG |
| 16S_GPbac_P6 | 213589 | CCAGGTCATAAGGGGCATGATGATTTGACGTCATCCCCACCTTCCTCCGG |
| 16S_GPbac_P7 | 213590 | CACCTTAGAGTGCCCAACTGAATGCTGGCAACTAAGATCAAGGGTTGCGC |
| 16S_GPbac_P8 | 213591 | ACCCAACATCTCACGACACGAGCTGACGACAACCATGCACCACCTGTCAC |
| 16S_GPbac_P9 | 213592 | GACGTCCTATCTCTAGGATTGTCAGAGGATGTCAAGACCTGGTAAGGTTC |
| 16S_GPbac_P10 | 213593 | ATTAAACCACATGCTCCACCGCTTGTGCGGGCCCCCGTCAATTCCTTTGA |
| 16S_GPbac_P11 | 213594 | CCGTACTCCCCAGGCGGAGTGCTTAATGCGTTAGCTGCAGCACTAAGGGG |
| 16S_GPbac_P12 | 213595 | ACTTAGCACTCATCGTTTACGGCGTGGACTACCAGGGTATCTAATCCTGT |
| 16S_GPbac_P13 | 213596 | TCGCTCCTCAGCGTCAGTTACAGACCAGAGAGTCGCCTTCGCCACTGGTG |
| 16S_GPbac_P14 | 213597 | ACGCATTTCACCGCTACACGTGGAATTCCACTCTCCTCTTCTGCACTCAA |
| 16S_GPbac_P15 | 213598 | ATGACCCTCCCCGGTTGAGCCGGGGGCTTTCACATCAGACTTAAGAAACC |
| 16S_GPbac_P16 | 213599 | ACGCCCAATAATTCCGGACAACGCTTGCCACCTACGTATTACCGCGGCTG |
| 16S_GPbac_P17 | 213600 | CCGTGGCTTTCTGGTTAGGTACCGTCAAGGTACCGCCCTATTCGAACGGT |
| 16S_GPbac_P18 | 213601 | ACAACAGAGCTTTACGATCCGAAAACCTTCATCACTCACGCGGCGTTGCT |
| 16S_GPbac_P19 | 213602 | CCATTGCGGAAGATTCCCTACTGCTGCCTCCCGTAGGAGTCTGGGCCGTG |
| 16S_GPbac_P20 | 213603 | GGCCGATCACCCTCTCAGGTCGGCTACGCATCGTCGCCTTGGTGAGCCGT |
| 16S_GPbac_P21 | 213604 | CTAATGCGCCGCGGGTCCATCTGTAAGTGGTAGCCGAAGCCACCTTTTAT |
| 16S_GPbac_P22 | 213605 | TTCAAACAACCATCCGGTATTAGCCCCGGTTTCCCGGAGTTATCCCAGTC |
| 16S_GPbac_P23 | 213606 | CCACGTGTTACTCACCCGTCCGCCGCTAACATCAGGGAGCAAGCTCCCAT |
| 16S_GPbac_P24 | 213607 | GCATGTATTAGGCACGCCGCCAGCGTTCGTCCTGAGCCAGGATCAAACTC |
| 23S_GPbac_P1 | 213608 | TGGTTAAGTCCTCGATCGATTAGTATCTGTCAGCTCCATGTGTCGCCACA |
| 23S_GPbac_P2 | 213609 | TATCAACCTGATCATCTTTCAGGGATCTTACTTCCTTGCGGAATGGGAAA |
| 23S_GPbac_P3 | 213610 | GGCTTCATGCTTAGATGCTTTCAGCACTTATCCCGTCCGCACATAGCTAC |
| 23S_GPbac_P4 | 213611 | GCAGAACAACTGGTACACCAGCGGTGCGTCCATCCCGGTCCTCTCGTACT |
| 23S_GPbac_P5 | 213612 | CAAATTTCCTGCGCCCGCGACGGATAGGGACCGAACTGTCTCACGACGTT |
| 23S_GPbac_P6 | 213613 | GTACCGCTTTAATGGGCGAACAGCCCAACCCTTGGGACTGACTACAGCCC |
| 23S_GPbac_P7 | 213614 | CGACATCGAGGTGCCAAACCTCCCCGTCGATGTGGACTCTTGGGGGAGAT |
| 23S_GPbac_P8 | 213615 | GGGGTAGCTTTTATCCGTTGAGCGATGGCCCTTCCATGCGGAACCACCGG |
| 23S_GPbac_P9 | 213616 | TTTCGTCCCTGCTCGACTTGTAGGTCTCGCAGTCAAGCTCCCTTGTGCCT |
| 23S_GPbac_P10 | 213617 | GATTTCCAACCATTCTGAGGGAACCTTTGGGCGCCTCCGTTACCTTTTAG |
| 23S_GPbac_P11 | 213618 | GTCAAACTGCCCACCTGACACTGTCTCCCCGCCCGATAAGGGCGGCGGGT |
| 23S_GPbac_P12 | 213619 | GCCAGGGTAGTATCCCACCGATGCCTCCACCGAAGCTGGCGCTCCGGTTT |
| 23S_GPbac_P13 | 213620 | ATCCTGTACAAGCTGTACCAACATTCAATATCAGGCTGCAGTAAAGCTCC |
| 23S_GPbac_P14 | 213621 | CCTGTCGCGGGTAACCTGCATCTTCACAGGTACTATAATTTCACCGAGTC |
| 23S_GPbac_P15 | 213622 | GCCCAGATCGTTGCGCCTTTCGTGCGGGTCGGAACTTACCCGACAAGGAA |
| 23S_GPbac_P16 | 213623 | ACCGTTATAGTTACGGCCGCCGTTTACTGGGGCTTCAATTCGCACCTTCG |
| 23S_GPbac_P17 | 213624 | CCTCTTAACCTTCCAGCACCGGGCAGGCGTCAGCCCCTATACTTCGCCTT |
| 23S_GPbac_P18 | 213625 | CCTGTGTTTTTGCTAAACAGTCGCCTGGGCCTATTCACTGCGGCTCTCTC |
| 23S_GPbac_P19 | 213626 | CAGAGCACCCCTTCTCCCGAAGTTACGGGGTCATTTTGCCGAGTTCCTTA |
| 23S_GPbac_P20 | 213627 | ATCACCTTAGGATTCTCTCCTCGCCTACCTGTGTCGGTTTGCGGTACGGG |
| 23S_GPbac_P21 | 213628 | TAGAGGCTTTTCTTGGCAGTGTGGAATCAGGAACTTCGCTACTATATTTC |
| 23S_GPbac_P22 | 213629 | TCAGCCTTATGGGAAACGGATTTGCCTATTTCCCAGCCTAACTGCTTGGA |
| 23S_GPbac_P23 | 213630 | CCGCGCTTACCCTATCCTCCTGCGTCCCCCCATTGCTCAAATGGTGAGGA |
| 23S_GPbac_P24 | 213631 | TCAACCTGTTGTCCATCGCCTACGCCTTTCGGCCTCGGCTTAGGTCCCGA |
| 23S_GPbac_P25 | 213632 | CGAGCCTTCCTCAGGAAACCTTAGGCATTCGGTGGAGGGGATTCTCACCC |
| 23S_GPbac_P26 | 213633 | TACCGGCATTCTCACTTCTAAGCGCTCCACCAGTCCTTCCGGTCTGGCTT |
| 23S_GPbac_P27 | 213634 | GCTCTCCTACCACTGTTCGAAGAACAGTCCGCAGCTTCGGTGATACGTTT |
| 23S_GPbac_P28 | 213635 | TCGGCGCAGAGTCACTCGACCAGTGAGCTATTACGCACTCTTTAAATGGT |
| 23S_GPbac_P29 | 213636 | AACATCCTGGTTGTCTAAGCAACTCCACATCCTTTTCCACTTAACGTATA |
| 23S_GPbac_P30 | 213637 | TGGCGGTCTGGGCTGTTTCCCTTTCGACTACGGATCTTATCACTCGCAGT |
| 23S_GPbac_P31 | 213638 | AAGTCATTGGCATTCGGAGTTTGACTGAATTCGGTAACCCGGTAGGGGCC |
| 23S_GPbac_P32 | 213639 | GCTCTACCTCCAAGACTCTTACCTTGAGGCTAGCCCTAAAGCTATTTCGG |
| 23S_GPbac_P33 | 213640 | TCCAGGTTCGATTGGCATTTCACCCCTACCCACACCTCATCCCCGCACTT |
| 23S_GPbac_P34 | 213641 | TTCGGGCCTCCATTCAGTGTTACCTGAACTTCACCCTGGACATGGGTAGA |
| 23S_GPbac_P35 | 213642 | TCTACGACCACGTACTCATGCGCCCTATTCAGACTCGCTTTCGCTGCGGC |
| 23S_GPbac_P36 | 213643 | TAACCTTGCACGGGATCGTAACTCGCCGGTTCATTCTACAAAAGGCACGC |
| 23S_GPbac_P37 | 213644 | GGCTCTGACTACTTGTAGGCACACGGTTTCAGGATCTCTTTCACTCCCCT |
| 23S_GPbac_P38 | 213645 | ACCTTTCCCTCACGGTACTGGTTCACTATCGGTCACTAGGGAGTATTTAG |
| 23S_GPbac_P39 | 213646 | CTCCCGGATTCCGACGGAATTTCACGTGTTCCGCCGTACTCAGGATCCAC |
| 23S_GPbac_P40 | 213647 | GTTTTGACTACAGGGCTGTTACCTCCTATGGCGGGCCTTTCCAGACCTCT |
| 23S_GPbac_P41 | 213648 | CTTTGTAACTCCGTACAGAGTGTCCTACAACCCCAAGAGGCAAGCCTCTT |
| 23S_GPbac_P42 | 213649 | CGTTTCGCTCGCCGCTACTCAGGGAATCGCATTTGCTTTCTCTTCCTCCG |
| 23S_GPbac_P43 | 213650 | CAGTTCCCCGGGTCTGCCTTCTCATATCCTATGAATTCAGATATGGATAC |
| 23S_GPbac_P44 | 213651 | GGTGGGTTTCCCCATTCGGAAATCTCCGGATCAAAGCTTGCTTACAGCTC |
| 23S_GPbac_P45 | 213652 | TGTTCGTCCCGTCCTTCATCGGCTCCTAGTGCCAAGGCATCCACCGTGCG |
| 16S:A1 | 213653 | AAACTAGATTCGAATATAACAAAACATTACATCCTCATCCAATCCCTTTT |
| 16S:A2 | 213654 | GCGGTGTGTGCAAGGAGCAGGGACGTATTCACCGCGCGATTGTGACACGC |
| 16S:A3 | 213655 | GCCTTTCGGCGTCGGAACCCATTGTCTCAGCCATTGTAGCCCGCGTGTTG |
| 16S:A4 | 213656 | GCATACGGACCTACCGTCGTCCACTCCTTCCTCCTATTTATCATAGGCGG |
| 16S:A5 | 213657 | CGGCATCCAAAAAAGGATCCGCTGGTAACTAAGAGCGTGGGTCTCGCTCG |
| 16S:A6 | 213658 | CAACCTGGCTATCATACAGCTGTCGCCTCTGGTGAGATGTCCGGCGTTGA |
| 16S:A7 | 213659 | AGGCTCCACGCGTTGTGGTGCTCCCCCGCCAATTCCTTTAAGTTTCAGTC |
| 16S:A8 | 213660 | CCAGGCGGCGGACTTAACAGCTTCCCTTCGGCACTGGGACAGCTCAAAGC |
| 16S:A9 | 213661 | TCCGCATCGTTTACAGCTAGGACTACCCGGGTATCTAATCCGGTTCGCGC |
| 16S:A10 | 213662 | TTCCCACAGTTAAGCTGCAGGATTTCACCAGAGACTTATTAAACCGGCTA |
| 16S:A12 | 213663 | CTCTTATTCCAAAAGCTCTTTACACTAATGAAAAGCCATCCCGTTAAGAA |
| 16S:A13 | 213664 | CCCCCGTCGCGATTTCTCACATTGCGGAGGTTTCGCGCCTGCTGCACCCC |
| 16S:A14 | 213665 | TTGTCTCAGGTTCCATCTCCGGGCTCTTGCTCTCACAACCCGTACCGATC |
| 16S:A16 | 213666 | CATTACCTAACCAACTACCTAATCGGCCGCAGACCCATCCTTAGGCGAAA |
| 16S:A17 | 213667 | AAACCATTACAGGAATAATTGCCTATCCAGTATTATCCCCAGTTTCCCAG |
| 16S:A18 | 213668 | AAGGGTAGGTTATCCACGTGTTACTGAGCCGTACGCCACGAGCCTAAACT |
| 23S:A1 | 213669 | ACCTAGCGCGTAGCTGCCCGGCACTGCCTTATCAGACAACCGGTCGACCA |
| 23S:A2 | 213670 | CGTTCCTCTCGTACTGGAGCCACCTTCCCCTCAGACTACTAACACATCCA |
| 23S:A3 | 213671 | CCTGTCTCACGACGGTCTAAACCCAGCTCACGTTCCCCTTTAATGGGCGA |
| 23S:A4 | 213672 | GGTGCTGCTGCACACCCAGGATGGAAAGAACCGACATCGAAGTAGCAAGC |
| 23S:A5 | 213673 | GGCTCTTGCCTGCGACCACCCAGTTATCCCCGAGGTAGTTTTTCTGTCAT |
| 23S:A6 | 213674 | AGGAGGACTCTGAGGTTCGCTAGGCCCGGCTTTCGCCTCTGGATTTCTTG |
| 23S:A7 | 213675 | CAAAGTAAGTTAGAAACACAGTCATAAGAAAGTGGTGTCTCAAGAACGAA |
| 23S:A8 | 213676 | GACTTATAATCGAATTCTCCCACTTACACTGCATACCTATAACCAAGCTT |
| 23S:A9 | 213677 | GTAAAACTCTACGGGGTCTTCGCTTCCCAATGGAAGACTCTGGCTTGTGC |
| 23S:A10 | 213678 | TCACTAAGTTCTAGCTAGGGACAGTGGGGACCTCGTTCTACCATTCATGC |
| 23S:A11 | 213679 | CGACAAGGCATTTCGCTACCTTAAGAGGGTTATAGTTACCCCCGCCGTTT |
| 23S:A12 | 213680 | AACTGAACTCCAGCTTCACGTGCCAGCACTGGGCAGGTGTCGCCCTCTGT |
| 23S:A13 | 213681 | CTAGCAGAGAGCTATGTTTTTATTAAACAGTCGGGCCCCCCTAGTCACTG |
| 23S:A14 | 213682 | TTAAAACGCCTTAGCCTACTCAGCTAGGGGCACCTGTGACGGATCTCGGT |
| 23S:A15 | 213683 | ACAAAACTAACTCCCTTTTCAAGGACTCCATGAATCAGTTAAACCAGTAC |
| 23S:A16 | 213684 | ATAATGCCTACACCTGGTTCTCGCTATTACACCTCTCCCCAGGCTTAAAC |
| 23S:A17 | 213685 | CAATCCTACAAAACATATCTCGAAGTGTCAGAAATTAGCCCTCAACGTCA |
| 23S:A18 | 213686 | CTTTGCTGCTACTACTACCAGGATCCACATACCTGCAAGGTCCAAAGGAA |
| 23S:A19 | 213687 | CAACCCACACAGGTCGCCACTCTACACAATCACCAAAAAAAAGGTGTTCC |
| 23S:A20 | 213688 | GGATTAATTCCCGTCCATTTTAGGTGCCTCTGACCTCGATGGGTGATCTG |
| 23S:A21 | 213689 | AGGGTGGCTGCTTCTAAGCCCACCTTCCCATTGTCTTGGGCCAAAGACTC |
| 23S:A22 | 213690 | GTATTTAGGGGCCTTAACCATAGTCTGAGTTGTTTCTCTTTCGGGACACA |
| 23S:A23 | 213691 | CCTCACTCCAACCTTCTACGACGGTGACGAGTTCGGAGTTTTACAGTACG |
| 23S:A24 | 213692 | CCCTAAACGTCCAATTAGTGCTCTACCCCGCCACCAACCTCCAGTCAGGC |
| 23S:A25 | 213693 | AATAGATCGACCGGCTTCGGGTTTCAATGCTGTGATTCCAGGCCCTATTA |
| 23S:A26 | 213694 | ACAACGCTGCGGGCATATCGGTTTCCCTACGACTACAAGGATAAAAACCT |
| 23S:A27 | 213695 | ACAAAGAACTCCCTGGCCCGTGTTTCAAGACGGACGATGCAACACTAGTC |
| 23S:A28 | 213696 | ACAATGTTACCACTGATTCTTTCGGAAGAATTCATTCCTTACGCGCCACA |
| 23S:A29 | 213697 | CTGGTTTCAGGTACTTTTCACCCCCCTATAGGGGTACTTTTCAGCATTCC |
| 23S:A30 | 213698 | CTCTATCGGTCTTGAGACGTATTTAGAATTGGAAGTTGATGCCTCCCACA |
| 23S:A31 | 213699 | ATCACCCTCTACGGTTCTAAAATTCCAAATAAAATTCGATTTATCCCACG |
| 23S:A32 | 213700 | TCTATACACCACATCTCCCTAATATTACTAAAAGGGATTCAGTTTGTTCT |
| 23S:A33 | 213701 | GCCGTTACTAACGACATCGCATATTGCTTTCTTTTCCTCCGCCTACTAAG |
| 23S:A34 | 213702 | GGGTTCCCAATCCTACACGGATCAACACAAAAAAAATGTGCTAGGAAGTC |
| 5S:A1 | 213703 | ACTACTGGGATCGAAACGAGACCAGGTATAACCCCCATGCTATGACCGCA |
| MM_16S_P10 | 213704 | GCGTATGCCTGGAGAATTGGAATTCTTGTTACTCATACTAACAGTGTTGC |
| MM_16S_P11 | 213705 | GATTAACCCAATTTTAAGTTTAGGAAGTTGGTGTAAATTATGGAATTAAT |
| MM_16S_P12 | 213706 | AGCTTGAACGCTTTCTTTATTGGTGGCTGCTTTTAGGCCTACAATGGTTA |
| MM_16S_P13 | 213707 | ATTATTCACTATTAAAGGTTTTTTCCGTTCCAGAAGAGCTGTCCCTCTTT |
| MM_16S_P14 | 213708 | CTTACTTTTTGATTTTGTTGTTTTTTTAGCAAGTTTAAAATTGAACTTAA |
| MM_16S_P15 | 213709 | AACCAGCTATCACCAAGCTCGTTAGGCTTTTCACCTCTACCTAAAAATCT |
| MM_16S_P7 | 213710 | AATACTTGTAATGCTAGAGGTGATGTTTTTGGTAAACAGGCGGGGTTCTT |
| MM_16S_P8 | 213711 | TTTATCTTTTTGGATCTTTCCTTTAGGCATTCCGGTGTTGGGTTAACAGA |
| MM_16S_P9 | 213712 | TTATTTATAGTGTGATTATTGCCTATAGTCTGATTAACTAACAATGGTTA |
| RN_16S_P4 | 213713 | AGTGATTGTAGTTGTTTATTCACTATTTAAGGTTTTTTCCTTTTCCTAAA |
| RN_16S_P5 | 213714 | TGGCTATATTTTAAGTTTACATTTTGATTTGTTGTTCTGATGGTAAGCTT |
| RN_16S_P6 | 213715 | TTTTTTTAATCTTTCCTTAAAGCACGCCTGTGTTGGGCTAACGAGTTAGG |
| RN_16S_P7 | 213716 | TGTTGGGTTAGTACCTATGATTCGATAATTGACAATGGTTATCCGGGTTG |
| RN_16S_P8 | 213717 | AGGAGAATTGGTTCTTGTTACTCATATTAACAGTATTTCATCTATGGATC |
| RN_16S_P9 | 213718 | TTTGTGATATAGGAATTTATTGAGGTTTGTGGAATTAGTGTGTGTAAGTA |
| MM_28S_P1 | 213719 | GCCGGGGAGTGGGTCTTCCGTACGCCACATTTCCCACGCCGCGACGCGCG |
| MM_28S_P10 | 213720 | ACCTCGGGCCCCCGGGGGGGGCCCTTCACCTTCATTGCGCCACGGCGGCT |
| MM_28S_P14 | 213721 | TCGCGTCCAGAGTCGCCGCCGCCGCCGGCCCCCCGAGTGTCCGGGCCCCC |
| MM_28S_P15 | 213722 | CGCTGGTTCCTCCCGCTCCGGAACCCCCGCGGGGTTGGACCCGCCGCCCC |
| MM_28S_P16 | 213723 | CGCCGACCCCCGACCCGCCCCCCGACGGGAAGAAGGAGGGGGGAAGAGAG |
| MM_28S_P17 | 213724 | GGGACGACGGGGCCCCGCGGGGAAGAGGGGAGGGCGGGCCCGGGCGGAAA |
| MM_28S_P18 | 213725 | GGCGCCGCGCGGAAAACCGCGGCCCGGGGGGCGGACCCGGCGGGGGAACA |
| MM_28S_P19 | 213726 | CCCCCACACGCGCGGGACACGCCCGCCCGCCCCCGCCACGCACCTCGGGA |
| MM_28S_P2 | 213727 | CACCCGCTTTGGGCTGCATTCCCAAGCAACCCGACTCCGGGAAGACCCGA |
| MM_28S_P20 | 213728 | TGGAGCGAGGCCCCGCGGGGAGGGGACCCGCGCCGGCACCCGCCGGGCTC |
| MM_28S_P21 | 213729 | CGAGGCCGGCGTGCCCCGACCCCGACGCGAGGACGGGGCCGGGCGCCGGG |
| MM_28S_P22 | 213730 | TCCCCGGAGCGGGTCGCGCCCGCCCGCACGCGCGGGACGGACGCTTGGCG |
| MM_28S_P23 | 213731 | TCCACACGAACGTGCGTTCAACGTGACGGGCGAGAGGGCGGCCCCCTTTC |
| MM_28S_P24 | 213732 | TCCCAAGACGAACGGCTCTCCGCACCGGACCCCGGTCCCGACGCCCGGCG |
| MM_28S_P25 | 213733 | CCGCCGCGGGGACGACGCGGGGACCCCGCCGAGCGGGGACGGACGGGGAC |
| MM_28S_P3 | 213734 | GCACCGCCACGGTGGAAGTGCGCCCGGCGGCGGCCGGTCGCCGGCCGGGG |
| MM_28S_P6 | 213735 | CCCACCGGGCCCCGAGAGAGGCGACGGAGGGGGGTGGGAGAGCGGTCGCG |
| MM_28S_P7 | 213736 | CCCGGCCCCCACCCCCACGCCCGCCCGGGAGGCGGACGGGGGGAGAGGGA |
| MM_28S_P8 | 213737 | TATCTGGCTTCCTCGGCCCCGGGATTCGGCGAAAGCGCGGCCGGAGGGCT |
| MM_28S_P9 | 213738 | CGCCGCCGACCCCGTGCGCTCGGCTTCGTCGGGAGACGCGTGACCGACGG |
| RN_28S_P12 | 213739 | GCGCCCCCCCGCACCCGCCCCGTCCCCCCCGCGGACGGGGAAGAAGGGAG |
| RN_28S_P14 | 213740 | CGAACCCCGGGAACCCCCGACCCCGCGGAGGGGGAAGGGGGAGGACGAGG |
| RN_28S_P16 | 213741 | CACCCGGGGGGGCGACGAGGCGGGGACCCGCCGGACGGGGACGGACGGGG |
| RN_28S_P17 | 213742 | GCCAACCGAGGCTCCTTCGGCGCTGCCGTATCGTTCCGCTTGGGCGGATT |
| RN_28S_P4 | 213743 | CCCGGGCCCCCGGACCCCCGAGAGGGACGACGGAGGCGACGGGGGGTGGG |
| RN_28S_P5 | 213744 | TGGGAGGGGCGGCCCGGCCCCCGCGACCGCCCCCCTTTCCGCCACCCCAC |
| RN_28S_P6 | 213745 | GGGAGAGGCCGGGGGGAGAGCGCGGCGACGGGTATCCGGCTCCCTCGGCC |
| RN_28S_P7 | 213746 | CGCTGCTGCCGGGGGGCTGTAACACTCGGGGGGGGGTGGTCCGGCGCCCA |
| RN_28S_P8 | 213747 | CGCCGCCGACCCCGTGCGCTCGGCTTCGCTCCCCCCCACCCCGAGAAGGG |
| | 213748 | CTCATCCCCACCCTTTTCAACGGATGTGGGTTCGGTCCTCCACTGCCTCT |
| | 213749 | AGCCGGGGCTTCTTAGTCAGGTACCGTCATTTTTTCTTCCCTGCTGATAG |
| | 213750 | TAGATGATCAACCTACCGGGTTAGAGTAGCCATCACACAAGGGTAGTATC |
| | 213751 | CAGATGGCGGCATTGTCACTGCTCCGTCTCCACGTCACTCCTGAAGGTAG |
| | 213752 | GGGAAGCAGGGTGGACCACCACCCAAGGCTAAATACTACCTGATGACCGA |
| | 213753 | ACTAAACTTCACTCCGCATCACGTCTTCCCATTGCCGCACGGTTTTTCCA |
| | 213754 | GTTCCTCCGCTTGTGCGGGCCCCCGTCAATTCCTTTGAGTTTCACCGTTG |
| | 213755 | GCCCCAGACAACCATCGCTGGGGTTGAGCTACCTCACTGCGTCCCTCCGC |
| | 213756 | CTTTCGTGCGGGTCGGAACTTACCCGACAAGGAATTTCGCTACCTTAGGA |
| | 213757 | CAGGCGTCAGCTCGTATACGTCATCTTTCGATTTAGCACAAACCTGTGTT |
| | 213758 | GGCTTCATGCTTAGATGCTTTCAGCACTTATCCCGTCCGCACATAGCTAC |
| | 213759 | ATTACCGCGGCTGCTGGCACGTAGTTAGCCGGGGCTTCTTAGTCAGGTAC |
| | 213760 | TTCACGCAAGATTTCTCGTGTCCCGCGCTACTCAGGATACCACTACGCTT |
| | 213761 | ATCTAAAGTCTTCTCGTTTAAAATACTGGGCTGTTACCATCTGTGGCGGA |
| | 213762 | GGGCTCTGACTTCTTGTAGGCATACGGTTTCAGGTTCTCTTTCACTCCGC |
| | 213763 | GCTATGGATCGTCGGTTTGGTGGGCCGTTACCCCGCCAACTGCCTAATCC |
| | 213764 | ATGACTTCAGCATGGGCGGTCATAACGCGGTACCAGAATATCAACTGGTT |
| | 213765 | TTTCAGTTCAGGCGGTTCCCCTCATATACCTATGTATTCAGTATATGATG |
| | 213766 | CGAAAGGGGAGACGGCACGGGCCCGGAGGTTAGCGCCCCAGGCCTCGGTT |
| | 213767 | TTTCGTCCCTGCTCGACTTGTAGGTCTCGCAGTCAAGCTCCCTTGTGCCT |
| | 213768 | CTCTTATCGATGACATCTCCTCTTAACCTTCCAGCACCGGGCAGGTGTCA |
| | 213769 | TCGTCCCTGACAACAGAGCTTTACGATCCGAAAACCTTCTTCACTCACGC |
| | 213770 | ACCCAACATCTCACGACACGAGCTGACGACAACCATGCACCACCTGTCAC |
| | 213771 | GTCCTCTCGTACTAAGGACAGAGCTCCTCAAATATCCTGCGCCCACGACA |
| | 213772 | TTATAGTTACGGCCGCCGTTTACCGGGGCTTCAATTCAGAGCTCTCACTC |
| | 213773 | CGTTTCTACGAGTTAGAACTCAAATAATCAAAGGGCCGTATTTCAACAGC |
| | 213774 | CACCAGTGTCGGTTTAGGGTACGGGCGGACCCGCCACCTCGCTCACGAAG |
| | 213775 | CGTCCATCCCGGTCCTCTCGTACTAGGGACAGCTCCTCTCAAATATCCTG |
| | 213776 | AGCTGACGCTCATGTTTCCAAGTCTCCCGCCTATCCTGTACATAGATTTC |
| | 213777 | CTCTTTTAATGAGTGGCTGCTTCTAAGCCAACATCCTGGTTGTCTAAGCA |
| | 213778 | ACAGCTTTTCTCGCCATCTTCCATCCCAGACTTCGGTACTAACTTCCCTC |
| | 213779 | CATAGACCTGTGTTTTTGCTAAACAGTTGCTTGAGCCTATTCTCTGCGGC |
| | 213780 | TCACGGTACTGGTTCACTATCGCTCACTCGTTTATATTTAGCCTTGGCGG |
| | 213781 | ACTCACCCTGCCCCGATTAACGTTGGACAGGAACCCTTGGTCTTCCGGCG |
| | 213782 | GGCTACAGTAAAGCTCCATGGGGTCTTTCCGTCTTGTCGCGGGTAACCGG |
| | 213783 | GTACGATTTGATGTTACCTGATGCTTAGAGGCTTTTCCTGGAAGCAGGGC |
| | 213784 | AAGTCATTGGCATTCGGAGTTTGACTGAATTCGGTAACCCGGTAGGGGCC |
| | 213785 | GGTTACCTTGTTACGACTTCACCCCAGTCATGAATCACAAAGTGGTAAGT |
| | 213786 | CCCTTCTCCCGTTGGCCTTAGAATCTTCTTCCTACCTACCTGTGTCGGTT |
| | 213787 | TACCTTCACTAAGGTTCTTTCCGACGCTAGCCCTAAAGCTATTTCGGGGA |
| | 213788 | CCCCCCTGCTTCCCACAGGGTTTCACGTGTCCCGTGGTACTCTGGATCAC |
| | 213789 | GACCGGCCTTCCCATGCCGTTCGGTTAACAGATTAAGTCTTAAAAGCAGT |
| | 213790 | TTCCTTTGACCCCCCCCCCCCCCCTCCCTATCCCCCCCCGCCCCCCCCCA |
| | 213791 | CCCCCTCAGTTCTCCAGCGCCCACGGCAGATAGGGACCGAACTGTCTCAC |
| | 213792 | CTTTGGGAGGCAACCGCCCCAGTTAAACTACCCGCCAGGCACTCTCCCCG |
| | 213793 | ACATGATCGGTTCACACACTCACCACCACACAAGACCTCAAAGAGACCCC |
| | 213794 | CCAGCACCGGGCAGGTGTCACCCCCTATACTTCGTCTTGCGACTTCGCAG |
| | 213795 | GTACCGCTTTAATGGGCGAACAGCCCAACCCTTGGGACTGACTACAGCCC |
| | 213796 | CCATTGCGGAAGATTCCCTACTGCTGCCTCCCGTAGGAGTCTGGGCCGTG |
| | 213797 | TTCTCTGCGGCTCATGTTTCCATGAGCACCCCTTATCCCTAAGTTACGGG |
| | 213798 | TTTGACTCATATCACACCTCACTGCTTAGACGTGCACTTCCAATCGCACG |
| | 213799 | CCGGTTTGCCCTCTTCCGCGTTCGCTCGCCACTACTTACGGAATCTCGTT |
| | 213800 | TACCTGATCGACTTGTCAGTCTCCCAGTCAAGCGCCCTTATGCCATTACA |
| | 213801 | TCCCAAGCTTCGGTGTATGATTTAGCCCCGTTAAATTTTCGGCGCAGGGT |
| | 213802 | CCTAGTCTTTTCAGTGCTCTACAAGCCGTGGTCATGGTTCGAGGCTGTAC |
| | 213803 | TCGGGGTGCTTTTCACCTTTCCTTCACAGTACTCGTACGCTATCGGTCTC |
| | 213804 | GGTCTGGGCTCTTTCCCTTTCGACTGCCCAACTTATCTCGTGCAGTCTGA |
| | 213805 | GCACTCCACAGCTCCTTCCGGTACTGCTTCTTCGCGTTAAGAATGCTCCT |
| | 213806 | GACTGCGAACCGTGAGCATTCGGAGTTCGTCAGGACTCGATAGGCGGTGA |
| | 213807 | GTAAACAGTCGCTTGGGTCTATTCTCTGCGGCCCATTCCTGGGCACTCCT |
| | 213808 | CCCACTTTCGTGCCTGCTCGACGTGTCTGTCTCGCAGTCAAGCCACCTTG |
| | 213809 | TTTCCCTGCGGCTCCGGGACTTTATCCCTTAACCTTGCCAGTATGCACAA |
| | 213810 | GGGCGCCTTCGCTTCGTAGCAGCTTTTCTCGCCAGCGTGAATTCAGCAGC |
| | 213811 | TTCCGCCTGACCTTAGCTCCCGACTAACCCTGAGCGGACGAACCTTCCTC |
| | 213812 | CTCTCAGGTCGGCTACTGATCGTCGGCTTGGTAGGCCGTTACCCCACCAA |
| | 213813 | CTTCCTCCGGCTACTTAGATGTTTCAGTTCACCGGGTTCCCCTCCATACG |
| | 213814 | TACCTGATCGACTTGTTAGTCTCCCAGTCAAGCGCCCTTATGCCATTACA |
| | 213815 | GCAACCGCCCCAGTTAAACTACCCGCCAGGCACTGTCCCTGAACAGGATG |
| | 213816 | TTCCTCGTGTCTCGCCGTACTCAGGATCCCATTAGGCTTCGATCGGATTT |
| | 213817 | ACGGATCGTCGCCTTGGTAGGCCTTTACCCCACCAACTAGCTAATGCACC |
| | 213818 | TGTCGGTTTGGGGTACGGGCGGCAACGCGCCTGACGCCGGGGCTTTTCTC |
| | 213819 | CGGTTTCCGTTCGCGCTGAGGGAACCTTTGGGCGCCTCCGTTACATTTTG |
| | 213820 | TTATAGTTACGGCCGCCGTTTACTGGGGCTTCAATTCAATGCTCTCACAT |
| | 213821 | TGTAGCATGCGTGAAGCCCTGGACGTAAGGGGCATGATGATCTGACGTCA |
| | 213822 | AGCACCGGGCAGGTGTCAGCACCTATACGTCAGCTCTCGCTTTCGCAGAT |
| | 213823 | GCTGATAGGACGCGACCCCATCCCACGCCGATAGAATCTTTCCCACAATC |
| | 213824 | GTTTCAGGTTCTATTTCACTCCCCTCCCGGGGTGCTTTTCACCTTTCCCT |
| | 213825 | CGGCTCCCATTCCGTGTCACCCCTGCGCTCACCTACCACGGCTACGCTCC |
| | 213826 | TAGAGGCTTTTCTTGGCAGTGTGGAATCAGGAACTTCGCTACTATATTTC |
| | 213827 | GGGGAATCTCGGTTGATTTCTTTTCCTCGGGGTACTTAGATGTTTCAGTT |
| | 213828 | CATACCAGAGGTTCGTCCACCCAGGTCCTCTCGTACTATGGGCAGGCCTC |
| | 213829 | CGCGGGTCCATCTTATACCACCGGAGTTTTTCACACTGAGCCATGCAGCT |
| | 213830 | CTCCCGCAACCCCGGCCACGCAACCCCCGACGGGTATCGCGCGCGGCCGG |
| | 213831 | TTCTCTGCGGCTCCATCTCTGGAGCACCCCTTCTCCCGAAGTTACGGGGT |
| | 213832 | GAACATCCGGCATTACCACCCGTTTCCAGGAGCTATTCCGGAGCATGGGG |
| | 213833 | AGGTCCCGGGGTCTTTTCGTCCTTCTGCGCTTAACGAGCATCTTTACTCG |
| | 213834 | GCTTCGGTGGCATGTTTTAGCCCCGGACATTTTCGGCGCAGGACCTCTCG |
| | 213835 | GCTTCAAAGCCTCCGACCTATCCTACACATCACGTGCCCAGATTCAATGA |
| | 213836 | TACTTTATTTCGCTCCACATCACGGCTTCGTCTCATGCACAGCGGATTTG |
| | 213837 | CATGGGGTCTTTCCGTCCTGTCGCGGGTAACCTGCATCTTCACAGGTACT |
| | 213838 | GACCTTCCTCTCAGAACCCCTACTGATCGTTGCCTTGGTGGGCCGTTACC |
| | 213839 | ATGTTTCAGTTCCCCGGGTTCCCCTCCATACGTTATGGATTGGCGTATGG |
| | 213840 | TTAACGCTTTCGCTTGGCCGCTTACTGTATATCGCAAACAGCGAGTATTC |
| | 213841 | CCACGGAAAACCACCTCCGCGGCCGGCTCCCATTCCGTGTCACCCCTGCG |
| | 213842 | TCGTAACTCGCCGGTTCATTCTACAAAAGGCACGCTCTCACCCATTAACG |
| | 213843 | AGGATGCGACGAGCCGACATCGAGGTGCCAAACCTCCCCGCCGATATGGA |
| | 213844 | TCCCCGGAGTACCTTTTATCCTTTGAGCGATGTCCCTTCCATACGGAAAC |
| | 213845 | CGGCTTCCCTACTTTAATTTCGGTCCCTTACGCCCGGGTCAACCAACGCC |
| | 213846 | CTGCTTCCAAGCCAACATCCTAGCTGTCTTAGCAGTCAGACTTCGTTAGT |
| | 213847 | GCTACTCATACCGGCATTCTCACTTCTATGCGTTCCAGCGCTCCTCACGG |
| | 213848 | GCCTTCGGTGTCTGCCTTATACCCGATTATTATCCATGCCCGGACCCTCG |
| | 213849 | CCGGCTTTCCCAAAACCGTTCCACTAACATTGCAGAATCTTAAATGCAGT |
| | 213850 | TACCTGTGTCGGTTTGCGGTACGGGCACCTTAGTATACACATAAGCTTTT |
| | 213851 | TGTTACGCACTCTTTCAAGGGTGGCTGCTTCTGAGCCAACCTCCTGGCTG |
| | 213852 | CTGGAGACCTTGGATATTCGGCCACAAGGATTCTCACCTTGTTCTCGCTA |
| | 213853 | CAGTAACCCGCAAGGCTGCACCTAAATGCATTTCGGGGAGTACGAGCTAT |
| | 213854 | AAACCTTGGATATTCGGCCTAGAGGATTCTCACCTCTATCTCGCTACTCA |
| | 213855 | CGCTTGTGCGGGCCCCCGTCAATTTCTTTGAGTTTTAGCCTTGCGACCGT |
| | 213856 | ACCGGGACACGTGATCCCACAACACCGGCAACGCAACCCCCGACGGGTAT |
| | 213857 | GCTTTTCTCGCCTTCAGCCAAGTGTGCTTCCCTACTCTAATTTCGGTCCC |
| | 213858 | CACTACTCACGGAGTATCCCTTCCTGCAGGTACTGAGATGTTTCACTTCC |
| | 213859 | GATTGGAATTTCTCCGCTACCCACAGTTCATCCGCTACCATTTCAACGGG |
| | 213860 | TTCCACGAGTCCCGCGCTACTCGGGAGACACCATCCATGGTGCACGCGCA |
| | 213861 | GTCTTTTCGTCCCATCGCGGGTAATCGGCATCTTCACCGATACTACAATT |
| | 213862 | CCGTACATCATCTCGATGGCATTCGGAGTTTGATATTCTTTGGTAAGCTT |
| | 213863 | GGGCTTGGCTACCCGGCTATAGACTTGGCAGTCTAACCGGTGCACCAGCG |
| | 213864 | ACTTTCGTTACTGCTCGACCCGTCAGTCTCGCAGTTAGGCTCGCTTCTGC |
| | 213865 | CTACTGTTTCTCCGCGTATACAACGCTCCCCTACCCAATCCATTACTGGA |
| | 213866 | ACTTATAGTCAGCGCCCCTTCTCCCGAAGTTACGGGGCCATTTTGCCGAG |
| | 213867 | CTTCCAAGCCAACATCCTAGCTGTCTTAGCAATCTGACTTCGTTAGTTCA |
| | 213868 | CCTCGGCAACTGGCGTTACCGATTCTCAGCCTCCCACCTATCCTGTACAT |
| | 213869 | CCATAACGGCTCCCATCATCACACCTCGCCATGCATGCCATGCGGATTTG |
| | 213870 | CGTGCAGGTCGGAACTTACCCGACAAGGAATTTCGCTACCTTAGGACCGT |
| | 213871 | CATCCAAACACTTTTCAACGTGTCCTGGTTCGGTCCTCCAGTGCGTTTTA |
| | 213872 | GCCCTAAAGCTATTTCGGGGAGAACCAGCTATATCCGGGTTCGATTGGAA |
| | 213873 | CAGTAAAGCTCTACGGGGTCTCTCCGTCCAGTCGCGGGTAATGGGCATCT |
| | 213874 | GGAACCTTTGGGCGCCTCCGTTACGCTTTAGGAGGCGACCGCCCCAGTCA |
| | 213875 | CCCGCCGTGTGTCTCCCGTGATAACATTCTCCGGTATTCGCAGTTTGCAT |
| | 213876 | CAGGTGTCAGCCCCTATACTTCATCTTTCGATTTGGCAGAGACCTGTGTT |
| | 213877 | GACTCTTCCCAGAGTCTTCTTCTATTCCCTTGGCTGCTTTATCGCAGTCC |
| | 213878 | GGCAACCCAACAACCCACACACCATCATCTTCAGCTACAGGACTATCACC |
| | 213879 | AGCACCGGGCAGGTGTCAGGCTATATACCTCATGTTTCCATTTCGCATAG |
| | 213880 | TTGCATACTATTAAGTTCAGCTCGGAAGGTGGATTTGCCTGCCTTCCTCA |
| | 213881 | CCGGCGGATTTGCCAACCGGACACCCTACACCCTTGGACCAGGTCAATTC |
| | 213882 | GCCGGTTATAACGGTTCATATCACCTTACCGACGCTTATCGCAGATTAGC |
| | 213883 | CTGATACAACCAGTATCGCTCCGTCCATTTGCGCAGCACCAGTAATCATG |
| | 213884 | TCTTTGAATGTATGGCTGCTTCTGAGCCAACATCCTAGTTGTCTTCGAGA |
| | 213885 | TGGATTCTCGCCCTCTTGTACTCATTTCGACTACGGGACTGTTACCCTCT |
| | 213886 | CAGTATCAACTGCAATTTTACGGTTGAGCCGCAAACTTTCACAACTGACT |
| | 213887 | TTCTCTGCGGCTTACCTTCGTAAGCACCCCTTCTCCCGAAGTTACGGGGT |
| | 213888 | ATTACTAGCGATTCCAGCTTCACGCAGTCGAGTTGCAGACTGCGATCCGA |
| | 213889 | CATAGACCTGTGTTTTTGCTAAACAGTTGCTTGAGCCTATTCTCTGCGGC |
| | 213890 | TATAAGTCGAGGCTGCACCTAAATGCATTTCGGGGAGTACGAGCTATCTC |
| | 213891 | TCAACCTGTTGTCCATCGCCTACGCCTTTCGGCCTCGGCTTAGGTCCCGA |
| | 213892 | GGGGTAGCTTTTATCCGTTGAGCGATGGCCCTTCCATGCGGAACCACCGG |
| | 213893 | ATTAACCTATGGATTCAGTTAATGATAGTGTGTCGAAACACACTGGGTTT |
| | 213894 | CCTCTTAACCTTCCAGCACCGGGCAGGCGTCAGCCCCTATACTTCGCCTT |
| | 213895 | AAAAAGCAAGCTCTCTCAAGTTCCGTTCGACTTGCATGTGTTAGGCGCGC |
| | 213896 | GGGCCCGTGTCTCAGTGCCCATGTGGGGGACCCTCCTCAGGCCGGCTATC |
| | 213897 | GACTTAACAAACCGCCTGCGTGCGCTTTACGCCCAGTAATTCCGATTAAC |
| | 213898 | CAACCTGTTGTCCATCGGCTACGCTTTTCAGCCTCACCTTAGGTCCCGAC |
| | 213899 | CACACACCACCACCACCCGAAAGCGGAGGCGGGGCGCGGGCAGATTGGTT |
| | 213900 | CCGTTCGACTTGCATGTGTTAAGCACGCCGCCAGCGTTCATCCTGAGCCA |
| | 213901 | GGCACCCTCTACGGCCAGGCCTTCAAGCCTGTTCCCCTGGCAAGCCGTTT |
| | 213902 | GCCCTTCAAAAGCGTCCCTGTGTTTAAATCTTCGGAGGTTACGGAATTTC |
| | 213903 | TCGTGGTGTGACGGGCGGTGTGTACAAGGCCCGGGAACGTATTCACCGCG |
| | 213904 | TCCCGGGGTTCTTTTCACCGTTCCTTCACAGTACTATGCGCTATCGGTCA |
| | 213905 | GACTGTTCGAGGTTAGACATCAAACGAGAACAGAGCGGTATTTCACCTTG |
| | 213906 | CACCTTAGAGTGCCCAACTGAATGCTGGCAACTAAGATCAAGGGTTGCGC |
| | 213907 | TATGGCACTTAAGCCGACACCTCACGGCACGAGCTGACGACAACCATGCA |
| | 213908 | TCTCGTCCATTGACCAATATTCCTCACTGCTGCCTCCCGTAGGAGTTTGG |
| | 213909 | TTTTCACCTTTCCCTCACGGTACTGGTTCGCTATCGGTCTCTCGGGAGTA |
| | 213910 | TTCCCCATTCAGAGATCTCCGGATCAATGGATATTTGCTCCTCCCCGAAG |
| | 213911 | TGAGCCAACATCCTGGTTGTCTGCGTATCTTCACATCGTTTTCCACTTAA |
| | 213912 | TCGGAGTTTGATATTCTTCGGTAGGCTTTGACGCCCCCTAGGAAATTCAG |
| | 213913 | CCTTCGGCTCCCCTATTCGGTTAACCTTGCTACAGAATATAAGTCGCTGA |
| | 213914 | GTCTGGACCGTGTCTCAGTTCCAGTGTGGCTGGTCATCCTCTCAGACCAG |
| | 213915 | TTATCCGTTCCGTACATAGCTGCCCAGCCGTGCCATTGGCATGACAACTG |
| | 213916 | TTCACAGTACTATGCGCTATCGGTCACTAAGGAGTATTTAGCCTTGCGGG |
| | 213917 | GACTCACCCGGGGACGACGAACGTGGCCCCGGAACCCTTGGTCATCCAGC |
| | 213918 | GGCAACTTCAACCTGCACATGGATAGATCACCCGGTTTCGGGTCTACGTA |
| | 213919 | ACCACGAATTCCGCCTGCCTCAACTGCACTCAAGATATCCAGTATCAACT |
| | 213920 | ACCACGCATTGCTGCATCCCAAGCTTCGGTTACATGCTTAGCCCCGTTAC |
| | 213921 | CCAGAGCTTTTCTCGCCTCCGTCCAAGCATGCTTCCCTACTAAATTTCAG |
| | 213922 | GCTGCACCTAAATGCATTTCGGAGAGAACCAGCTATCACGGAATTTGATT |
| | 213923 | CCTGGTTCGGGCCTCCAGTGAGTTTTACCTCACCTTCACCCTGCTCATGG |
| | 213924 | ACTCACCCGGGGACGACGAACGTGGCCCCGGAACCCTTGGTCATCCAGCG |
| | 213925 | AACATCCTGGTTGTCTGTGCAATTCCACATCCTTCTCCACTTAACGTGAA |
| | 213926 | CTACGACTTCTCCCCATACAGAACGCTCTCCTACCATACATTAGATGTAT |
| | 213927 | CACACTTAGCCCCGGACAACCATCACCGGGGATGAGCTACCTCACTGCGT |
| | 213928 | GGGCGACCCTCCAACAGCGGCGGAACACATTTCGACTACGGGACTCTCAC |
| | 213929 | CTCCGGTGCTTAACCTTGCCAGTGAGCGCAACTCGCCGGACCGTTCTACA |
| | 213930 | TTCGCAGGCTTACAGAACGCTCCCCTACCCAACAACGCATAAGCGTCGCT |
| | 213931 | CCGTCAAGCCATGGGAGCCGGGTGTACCTAAAGTCGGTAACCGCAAGGAG |
| | 213932 | TTACCTACACCATCACCTACACGCTTACACCAACAATCCACTAAGCGGCA |
| | 213933 | GCGTACACCTGCAGCCTATCTACCTCGTAGTCTTCAAGGGGTCTTACCTG |
| | 213934 | GCCGTCGCCCGTTAGTACCGGTCGGCTCCACCCCTCGCGGGGCTTCCACC |
| | 213935 | CACAGTGCTGTGTTTTTAATAAACAGTTGCAGCCAGCTGGTATCTTCGAC |
| | 213936 | CTGTTATCCCCAGGGTAGCTTTTATCCGTTGAGCGACGGCATTTCCACTC |
| | 213937 | ACTTAGATGCTTTCAGCACTTATCCAATCCCGACTTAGATACCCGGCAAT |
| | 213938 | GCTTGCGCTAACCTCTCCTCTTAACCTTCCAGCACCGGGCAGGCGTCAGC |
| | 213939 | ACCTATCCTGTACATGTGGTACAGATACTCAATATCAAACTGCAGTAAAG |
| | 213940 | CTCCACCAGACTAAAACGAGGCTAGCCCTAAAGCTATTTCGAGGAGAACC |
| | 213941 | CCCGGCTTACCTTGGGCGGACGAACCTTCCCCAAGAAACCTTAGATTTTC |
| | 213942 | GCAGAACAACTGGTACACCAGCGGTGCGTCCATCCCGGTCCTCTCGTACT |
| | 213943 | GACCAGGTCGATTCCATTGCCTGGCCCGGCTACCTTCCTGCGTCACACCT |
| | 213944 | CTCTGAGACTTCAAATGTGTCCCTGTGCTTAACTCTTTTGGTGGTGACGG |
| | 213945 | ACCTCGCGGTACGCCTTCGACGCTGACTGGAATGCTCCCCTACCGATCAT |
| | 213946 | CGTCCATCCTGAGGGAACCTTTGGGCGCCTCCGATACCCTTTCGGAGGCG |
| | 213947 | CACCTATCGGTCTCTCCTTAGGTCCCGACTAACCCAGGGCGGACGAGCCT |
| | 213948 | CGCTCGCCGCTACTAAGGAAATCGATGTTTCTTTCTCTTCCTCCGGCTAC |
| | 213949 | CGCGAGTCCATCTTCAAGCGATAAAATCTTTGATATCAAAACCATGTGGT |
| | 213950 | TGACTGGAGTTTGTCCAGCCGGGTTTCCCCATTCAGAGATCTGCGGATCA |
| | 213951 | CCTACTTAGCTACCCGGCTATGCCCCTGGCGGAACAACCGGTGCACCAGC |
| | 213952 | ACGCTTAAACCGGGACAACCGTCGCCCGGCCAACATAGCCTTCTCCGTCC |
| | 213953 | GATTTGCCTGGGATAATCAACATCTACACCCTTTAACGGACTATTCCGTC |
| | 213954 | CTAATGCGCCGCGGGTCCATCTGTAAGTGGTAGCCGAAGCCACCTTTTAT |
| | 213955 | GGATCTTAGCACTCGCAGTCTGACTGCCGACCATAAATCAATGGCATTCG |
| | 213956 | ACCTATCCTGTACATGTGGTACAGGTACTCAATATCAAACTGCAGTAAAG |
| | 213957 | TCACCGGGGATGAGCTACCTCACTGCGTCCCTCCGCAGCTTGCCTACTAC |
| | 213958 | GCCATGCAGATTCTCACTGCATTCGCGCTACTCATTCCGGCATTCTCACT |
| | 213959 | CTTCACCTCACATACGACGCTCCCCTACCCCTGACAATTACTTGTCAAGC |
| | 213960 | CCCTACTGATCGTCGCCTTGGTGGGCCGTTACCCCGCCAACAAGCTAATC |
| | 213961 | ACGCATTCGGAGTTTGTCAAGACTTGATAGGCGGTGAAGCCCTCGCATCT |
| | 213962 | ACATTTTAGGAGGCGACCGCCCCAGTCAAACTGCCCGTCAGACACTGTCT |
| | 213963 | GGTGGGTTTCCCCATTCGGAAATCTCCGGATCAAAGCTTGCTTACAGCTC |
| | 213964 | CTCATCCCCACCCTTTTCAACGGATGTGGGTTCGGTCCTCCATTGCCTTT |
| | 213965 | AGGTCACTTGGTTTCGGGTCTACATCTACGTACTTAACCGCCCTTTTCAG |
| | 213966 | ACACACTCACCACACCACCACAACATCAAAGACATCACAATGGCAGGCTC |
| | 213967 | TGACAACTGGTGCACCAGAGGTGCGTCCATCCCGGTCCTCTCGTACTAGG |
| | 213968 | TCTGCCTCTGCACATTGCTCCTCTACCGCGCATCTTCTTCAGACGCACCC |
| | 213969 | CTTTTCTCGACAGTACGGGATCACCAACTTCACCAATTAAGGCTACGCAT |
| | 213970 | CCCTCATGTCACTATTTATTCATGACATGATGACACGCTGTTAACGTGCC |
| | 213971 | GTACGCAGTCACACGCCTAAGCGTGCTCCCACTGCTTGTACGTACACGGT |
| | 213972 | GGCGACCACCCCAGTCAAACTACCCACCAAGCAATGTCCGCGCATAGCGC |
| | 213973 | GACTTAGTCCCAATCACGAGCCTCACCTTAGACGGCTCCATCCCACAAGG |
| | 213974 | GCGCTTATGCGGTATTAGCAGTCATTTCTAACTGTTATCCCCCTGTATAA |
| | 213975 | CGCTTTCACTGCGGCTACGTGTCTCGTGACACTCAACCTCGCCAGTGACG |
| | 213976 | ATGCTTTTCGCTTACAGGACTATAACCTTCTTTGGTGTGCCTTCCCATAC |
| | 213977 | CGACTAACCCAGGGCGGACGAGCCTTCCCCTGGAAACCTTAGTCTTACGG |
| | 213978 | TAGGACCCGACTAACCCTGATCCGATTAGCGTTGATCAGGAAACCTTAGT |
| | 213979 | ACAGCTTTTCTCGTCTCTTTCCAAACTGACTTCCGCTTACGCGTCCCTTA |
| | 213980 | TAAGACTTGCTCTCGCTGCGGCTTCAGACCTTAAGTCCTTAACCTTGCCA |
| | 213981 | CTCTCAAACCAGCTATGGATCGTCGGCTTGGTAGGCCATTACCCCACCAA |
| | 213982 | GGAATTTCTCCCCTATCCACACGTCATCTCCACCCTTTTCAACGGATGTG |
| | 213983 | CCGGTCCATGGTCGGTACGGGAATATCCACCCGTTCATCCATTCGACTAC |
| | 213984 | CCCCCGACCGGTTTCACGGCCGCAGGTTAGAATTCCAGAAACCTAAGGGC |
| | 213985 | AAGTTTCGGTGGCTACGGAATTTCAACCGTATGTGCATCGACTACGCCTC |
| | 213986 | TGCGCTCCCTTTACACCCAGTAAATCCGGATAACGCTTGCCCCCTACGTA |
| | 213987 | ATTTCGCCTACGGGACTGTCACCCTCTATGGTCCACCTTTCCAGGTGAGT |
| | 213988 | GCTTCGGTGGCATGTTTTAGCCCCGGACATTTTCGGCGCAGGACCTCTCG |
| | 213989 | GACATGTCTCCACATCATTCAGTTGCAATTCAAGCCCGGGTAAGGTTCCT |
| | 213990 | CGATAACTGGCACACCAGAGGTGCGTCCTTCCCGGTCCTCTCGTACTAGG |
| | 213991 | AACGCTTATCGGTGCGGACCTCCATCCCGTGTTACCGGGACTTCATCCTG |
| | 213992 | CCACTCCGTCGATGTGAACTCTTGGGAGTGATAAGCCTGTTATCCCCAGG |
| | 213993 | GCCGCCTTTTCAACGGAGGTCGGTTCGGCCCTCCATGGAGTTTTACCTCC |
| | 213994 | ACCGTTATAGTTACGGCCGCCGTTTACTGGGGCTTCAATTCGCACCTTCG |
| | 213995 | AGGTGTTCTCATGTGGGTTTCCCCATTCAGAGATCTGCGGGTCAATGGAT |
| | 213996 | AGCCTGTTCCCCTGGCAAGCCGTTTTATGACTCCCGCCCGGTCCGTCGGA |
| | 213997 | GCTGACCTACTACGAGGGGGGATCCCAACGCGCCCGCGCCGCGACCCCCC |
| | 213998 | GTTATCCCCCTGTATGAGGCAGGTTACCCACGCGTTACTCACCCGTCCGC |
| | 213999 | CGGACATCTTCGGCGCACAATCACTCGACCAGTGAGCTATTACGCACTCT |
| | 214000 | TGCTTGATGCCCGATTATTATCCACGCCAAACTCCTCGACTAGTGAGCTG |
| | 214001 | CTCCATTCGGAAATCTGCGGATCAAAGCCTACTTACGGCTCCCCGCAGCT |
| | 214002 | GCTGTTGGTCCGGATTGTTCTCCTTTAGGACATGGACCTTAGCACCCATG |
| | 214003 | TGCTGGCACGGAGTTAGCCGTCACTTCCTTGTTGAGTACCGTCATTATCT |
| | 214004 | GCTATCGGTCAGACAGGTATGCTTAGACTTACCCAACGGTCTGGGCTGAT |
| | 214005 | TATTCCTCACTGCTGCCTCCCGTAGGAGTTTGGACCGTGTCTCAGTTCCA |
| | 214006 | TCCCGCTGGCCTTAGAATTCTCTTCCTGTCCACCTGTGTCGGTTTGCGGT |
| | 214007 | CGACTATTGTCCTCGGCTTAGGTCCCGACTTACCCTGAGAGGACGAGCCT |
| | 214008 | GGTCCTTTTCACCTTTCCTTCACAGTACTATGCGCTATCGGTCACTAAGT |
| | 214009 | TCGGCTACTGATCGTCGCCTTGGTAGGCCGTTGCCCTGCCAACTAGCTAA |
| | 214010 | CTTGGGAGTATGTTTACACGCACTATTACCGTTTTCCGAGGAAATTGGTA |
| | 214011 | CACACAACCCCTACCAGGTATCACATGCACACGGTTTAGCCTCATCCACG |
| | 214012 | CCACGGCTTCGGTGTTGTGTTTTAGCCCCGGACATTTTCGGCGCAGGGCC |
| | 214013 | CCACCTTCCTCCAGTTTATCACTGGCAGTCTCCTTTGAGTTCCCGGCCGG |
| | 214014 | AGCTTTCGGGGAGAACCAGCTATCTCCCGGTTTGATTGGCCTTTCACCCC |
| | 214015 | CGAGCCTTCCTCAGGAAACCTTAGGCATTCGGTGGAGGGGATTCTCACCC |
| | 214016 | CCCAGGGCTAGATCATCCCGCTTCGGGTCCAGGACAAGCGACTGAAAACG |
| | 214017 | AAAATCATGGGAAATCTCATCTTGAGGGGGGCTTCGCACTTAGATGCTTT |
| | 214018 | ATCCTGTACAAGCTGTACCAACATTCAATATCAGGCTGCAGTAAAGCTCC |
| | 214019 | TTAGCAGGTGGTCCGGATTCTTCTCCTCTCGGGCACGGACCTTAGCACCC |
| | 214020 | GTCCGTTTACGGTACGGGTACCTCAAGGATAAGTTTAGCGGGTTTTCTAG |
| | 214021 | CACTGGCGTGCTGCCTTCTCTGCCTCCCACCTATCCTGTACATGAAATAC |
| | 214022 | TGCGGTATTAGCAGTCATTTCTAACTGTTATCCCCCTGTATAAGGCAGGT |
| | 214023 | GCTATCGGTCAGACAGGTATGCTTAGACTTACACCACGGTCGGTGCGGAT |
| | 214024 | TTTACTCCTTTCGGATGGGATATCTCATCTTGAGGGGGGCTTCACGCTTA |
| | 214025 | TGGCCGGTCGCCCTCTCAGGCCGGCTACCCGTCGAAGCCTTGGTGAGCCG |
| | 214026 | AAGCCTGTTCCCCTGGCAAGCCGTTTTATGACTCCCGCCCGGCCCGTCGG |
| | 214027 | AAGGTTAAGCCTCACGGTTCATTAGTACCGGTTAGCTCAACGCATCGCTG |
| | 214028 | GACATCATACTAACGCGCCCTATTAAGACTCGGTTTCCCTACGGCTCCGT |
| | 214029 | TGTGTTTTTGTTAAACAGTTGCCTGGACCGATTCTCTGCGCCTCAAGTCG |
| | 214030 | GCCCCAGTCAAACTACCCACCAGACACTGTCCGCAACCCGGATTACGGGT |
| | 214031 | GCGTCACACCTGTTAATGCGCTTGCCTTACCGGTTCAGGTCCCGCGCTCC |
| | 214032 | GCGATGGCCCTTCCATGCGGAACCACCGGATCACTAAGCCCGACTTTCGT |
| | 214033 | AAGCTCCATGGGGTCTTTCCGTCTAGTCGCGGGTAACCGGCATCTTCACC |
| | 214034 | CGCTAGCCCTAAAGCTATTTCGGAGAGAACCAGCTATCTCCAAGTTCGTT |
| | 214035 | TCCCATCCGCACTTCGCTTCCCTGCTATGCCGTTGGCACGACAACAGTTG |
| | 214036 | TTTCACTCCCCTCCCGGGGTCCTTTTCACCTTTCCTTCACAGTACTCTGC |
| | 214037 | CGTCCTCGGCTTAGGCCCCGACTTACCCTGGGCGGATGAACCTTCCCCAG |
| | 214038 | CGACATCGAGGTGCCAAACCTCCCCGTCGATGTGGACTCTTGGGGGAGAT |
| | 214039 | TACCTGATCGACTTGTCAGTCTCCCAGTCAAGCGCCCTTATGCCATTACA |
| | 214040 | CTTCCAAGCCAACATCCTAGCTGTCTTAGCAATCTGACTTCGTTAGTTCA |
| | 214041 | ACGCCTTAACCATGTGAAGGGTAGATTTTCTGACCCCTTCGGCCTGAACG |
| | 214042 | CTCAAGGATTAAGTTTAGCGGATTTTCTCGGGAGTATGTTTACACGCACT |
| | 214043 | CCCCATCCATCACCGATAAATCTTTAATCTCTTTCAGATGTCTTCTAGAG |
| | 214044 | ATACTTTGGGACCTTAGCTGTGGGTCTGGGCTGTTTCCCTTTTGACAATG |
| | 214045 | CGCCCATAGGCGGTGCCGGCCCATGACGGCCGGCGGGTTCCCCCATTCGG |
| | 214046 | AAAATCATGGGAAATCTCATCTTGAGGTGGGCTTCGCACTTAGATGCTTT |
| | 214047 | ACAACTTGATACCCGATTATTATCCACGCCCGACTCCTCGACTAGTGAGC |
| | 214048 | CTGAGTTTGATAAGCTTCGCTAACCTCTCGGCCGCTAGGCTATTCAGTGC |
| | 214049 | GCCCAGATCGTTGCGCCTTTCGTGCGGGTCGGAACTTACCCGACAAGGAA |
| | 214050 | TTATAGTTACGGCCGCCGTTCACTGGGGCTTCGGATCACTGCTTCAGATC |
| | 214051 | GGCATTGTCCCACCGCCGGGTCACGGCGGCTGGTTAGAAACCCAATACTG |
| | 214052 | GTCCACACATTTAGCCCCAGACAACCATCGCTGGGGTTGAGCTACCTCAC |
| | 214053 | TCTCACGACGTTCTGAACCCAGCTCGCGTGCCGCTTTAATGGGCGAACAG |
| | 214054 | ATGCGACGAGCCGACATCGAGGTGCCAAACCTCCCCGTCGATGTGAACTC |
| | 214055 | CCTGTGTCGGTTTAGGGTACGGGCAGTTTGAACCTCGCGCCGATGCTTTT |
| | 214056 | CGATATTGCAAGGGTGGTATCCCAACAGCGCCTCCTCAGAGACTGGCGTC |
| | 214057 | CCCCCGACCGGATTCACGGCCGCAGGTTAGAATTTCAGCACCTCAAGAGT |
| | 214058 | TCAGATGGCGGCATTGTCACTACTGCGTCTCCACATCACTCCTGGAGGTA |
| | 214059 | CTTTTCGTCCCATCGCGGGTAATCGGCATCTTCACCGATACTACAATTTC |
| | 214060 | ACAACGAATTCCGCCAACTTCCCGCGCACTCAAGCCCTCCAGTTCGCGCT |
| | 214061 | CCCGAAGTTACGGGGCCAATTTGCCGAGTTCCTTAACAACCCTTCTCCCG |
| | 214062 | TCAAGGGGGTTTACTTCTTTCGAATGGGATATCTCATCTTAAGGGGGGCT |
| | 214063 | CTTCACAGTACTATACGCTATCGGTCACTGGGTAGTATTTAGGGTTGGAG |
| | 214064 | ATTCCGTCAGACGGCCGGACTGTCACTTCTCCGTCACCACATCGCTCTCT |
| | 214065 | CGGTACTGGTTCACTATCGGTCACTAGGGAGTATTTAGGGTTGGGAGATG |
| | 214066 | AGCTGATGGTCCGGATTCTTCTCCTTTAGGACATGGACCTTAGCACCCAT |
| | 214067 | CGTATTACCGCGGCTGCTGGCACGGAATTAGCCGGTCCTTATTCATAAGG |
| | 214068 | ACGGGTTAGCCTCGCCACGCACCACTGACTCGCAGACTCATTTTTCGATA |
| | 214069 | ACGGCGTGGACTACCAGGGTATCTAATCCTGTTCGCTCCCCACGCTTTCG |
| | 214070 | TGCGCATTCGGAGTTTATCAAGACTTGATAGGCGGTGAAGCCCTCGCATC |
| | 214071 | CTGTTGTCCATCGGCTACGACTCTCGTCCTCACCTTAGGCCCCGACTTAC |
| | 214072 | GGCTCACGCCTCACCTTCGACGCGGAGTGGAATGCTCCCCTACCGATGTT |
| | 214073 | GATGTTTCAGTTCAGGCGGTTCCCTCGATATACCTATTTTTAAGTTCAGT |
| | 214074 | CATTGTCTAAGATTCCCCACTGCTGCCTCCCGTAGGAGTCTGGGCCGTGT |
| | 214075 | TCACAGTACTATGCGCTATCGGTCACTAAGTGGTATTTAGCCTTAGGGGG |
| | 214076 | GTAGTATTTAGGCTTGGAGGATGGTCCCTCCTGCTTCCCACAGGGTTTCA |
| | 214077 | TTGGGACCTTAGCTGCGGGTCTGGGCTCTTTCCCTTTTGACTATCCAACT |
| | 214078 | CAGCTTGGTGGCGCAGAACTAAGCATTTGACTCAGTCCTCACCTCACTGC |
| | 214079 | ACCAAGTACAGGAATATTAACCTGTTTCCCATCGACTACGCCTTTCGGCC |
| | 214080 | AAGCCCGCTTGTGCGATTACACTCGACACCCGATTGCCAACCGGGCCGAG |
| | 214081 | CCTTAAATACGCACAACCATCGGCGCACTGCAGCTACCTGTCTGCGTCAC |
| | 214082 | CTACCCAGCGATGCCTTTGGCAAGACAACTGGTACACCAGCGGTAAGTCC |
| | 214083 | CCTGTGTCGGTTTACGGTACGGGCGCATGGCAAACAATAGCGGCTTTTCT |
| | 214084 | CCGCGCTTACCCTATCCTCCTGCGTCCCCCCATTGCTCAAATGGTGAGGA |
| | 214085 | GGCTCTCTGTACTGTCAGGTTTCAGCAAGGACTAACTCTTAATCTGCCCC |
| | 214086 | GGATCACCGGATTCGGGCCGTAAGGCCCCCATCATCGCGCCTCGCCCCGA |
| | 214087 | TGGTCTCCGCTCGTTCAGACAAGGTTTCACGTGTCTCGTCCTACTCTGGA |
| | 214088 | CAATCCCACTTTATGCCACCGGATCACTAAGTCCTACTTTCGTACCTGCT |
| | 214089 | GTCACCAAGTAGTATTTAGCCTTGGGGGGTGGGCCCCCCGTCTTCCCACC |
| | 214090 | ATCCCCGGAGTACCTTTTATCCGTTGAGCGATGGCCCTTCCATTCAGAAC |
| | 214091 | TACCTCTCACGGTGACCATCCGACGCGGCACCTAAATGCCTTTCGGGGAG |
| | 214092 | CCGTACTCCCCAGGCGGAGTGCTTAATGCGTTAGCTGCAGCACTAAGGGG |
| | 214093 | ATCACCAGTTTTACCCTAGGGCGCTCCTTGCGGTTACGCACTTCAGGTAC |
| | 214094 | GGAGGGCACCTTTAGAAGCCTCCGTTACGCTTTTGGAGGCGACCACCCCA |
| | 214095 | CTGGAGACCTTGGATATTCGGCCACAAGGATTCTCACCTTGTTCTCGCTA |
| | 214096 | GGGCTTTCACCCTCTTTGGCTGGCTTTCCCAAAACCATTCTGCTAGGATC |
| | 214097 | GTGGGATTGGCTTAACCTCGCGGTTTCGCTGCCCTTTGTTCTGTCCATTG |
| | 214098 | ATGCTACGCAGAGAAGTCCGGATATCAATGCCAGACTAGAGTAAAGCTCC |
| | 214099 | TCCGTATACTCTCAGGTTCGACTCTCCCCGCGGATTTGCCTACGGGAATC |
| | 214100 | CTGGACCTATTCTCTGCGCCTCACATTGCTGTGAGGACCCTTTATCCCGA |
| | 214101 | TTAGCAGGTGGTCCGGATTCTTCTCCTCTCGGGCACGGACCTTAGCACCC |
| | 214102 | GCCTGTACACCTGCATCCTATCAACGTCATAGTCTTTGACGACCCTGAGA |
| | 214103 | AGACTCCAATCCGGACTACGACGCACTTTATGAGGTCCGCTTGCTCTCGC |
| | 214104 | GGTTTGCCCTCCTGCCTCTTCGCTCGCCGCTACTGAGGCAATCGCTCTTG |
| | 214105 | ACCTTTCCCTCACGGTACTGGTACGCTATCGGTCAGACAGGTATGCTTAG |
| | 214106 | CCGGTCCTCTCGTACTAGGGACAGCTCCCATCAAATATCCTGCGCCCACG |
| | 214107 | CCATTGGCATGACAACCCGAACACCAGTGATGCGTCCACTCCGGTCCTCT |
| | 214108 | ATGTGCTTGTAAGCACAGAGTTTCAGGTTCTTTTCACTCCCCTCCCGGGG |
| | 214109 | CCCTTCTCCCGAAGTTACGGGGTAATTTTGCCGAGTTCCTTAACAACCCT |
| | 214110 | CCTGAGTCGGTTTAGGGTACGGGCGCGTTATGCCCTCACGTCGAGGCTTT |
| | 214111 | ATCTGGGCTGTTTCCCTTTCGACAATGAAACTTATCTCACACTGTCTGAC |
| | 214112 | CGTATTTCAAGGATGGCTCCACAAACACTGGCGTGCCTGCTTCAAAGCCT |
| | 214113 | GGTCATTGCCTGCTTGCGGCTGACCATGGCTTATCGCAGCTGACCACGTC |
| | 214114 | CCTGGCGCGGGTAACCAGCATCTTCACTGGTACTTCAATTTCACCGGGTG |
| | 214115 | GTAACTCACAAGGCTGCACCTAAATGCATTTCGGGGAGTACGAGCTATCT |
| | 214116 | GTCGGTTTGGGGTACGGGCGGCCATAGCCCTCACGCCGAGGCTTTTCTCG |
| | 214117 | CACCGTCTATGGTCCCATTTTCCAAAGGGTTCTACTCATGAAATGTCTTG |
| | 214118 | CCGGCAACGCAACCCCCGACGGGTATCACGCGCAACCGGTTTGGTCTGAT |
| | 214119 | TTATCCTTCTGTGTCACTGCTTCATTCCATCGGTAGTGCAGGAATCTACA |
| | 214120 | CAGAGCACCCCTTCTCCCGAAGTTACGGGGTCATTTTGCCGAGTTCCTTA |
| | 214121 | ATACTATCAGGTTCGATTCTCATGGTGGATTTGCCTGCCAAGATCAACAT |
| | 214122 | CTTACGGGGCTTTCACCCTCTCTGGCCGGCTTTCCCAAAACCGTTCTGCT |
| | 214123 | GACCGGCCTTCCCATGCCGTTCGGTTAACAACTTAAGTCCTAAATGCGGT |
| | 214124 | CGTTTATCCGATCCGTACGTAGTTGCCCAGCTATGCTCCTGGCGGAACAA |
| | 214125 | GTATCTAATCCTGTTTGATACCCACACTTTCGAGCATCAGCGTCAGTTAC |
| | 214126 | GGTGCTTGTAAACACAAGGTTTCAGGTTCTTTTTCACTCCCCGTCAGGGG |
| | 214127 | GTAGGCGCACGGTTTCAGGAACTCTTTCACTCCCCTCCCGGGGTGCTTTT |
| | 214128 | ACTTCTGAGTTCGGCATGGGGTCAGGTGGGACCACCGCGCTACGGCCGCC |
| | 214129 | TTCCGTGTTCGGTATGGGAACGGGTGTGACCTCTTCGCTATCGCCACCAA |
| | 214130 | TCGCCTTAGGACCCGACTCACCCGGGGACGTTAACCGTGGCCCCGGAACC |
| | 214131 | CACTCACCCACAACCATGGGCTCCCCATCATGCCTCAACCTTCACGCCCA |
| | 214132 | CTCCGAGACTTCATATGTGTCCCTGTGTTTAACTCTTTTGGTGGTGACGG |
| | 214133 | AAAATTCCCTACTGCTGCCTCCCGTAGGAGTTTGGGCCGTGTCTCAGTCC |
| | 214134 | GACCAGGTAAGGTTCTTCGCGTTGCATCGAATTAAACCACATGCTCCACC |
| | 214135 | CGAAGTTTGATAGGGTTCGGTAAGCTTTGTGGCCCCCTAGCCCATTCAGT |
| | 214136 | AGGCTTGCGCCGCCGCTTCGCCCCGATGGGGACGCTCTCCTACCCAGCGT |
| | 214137 | CGAACAGAGCGGTATTTCACCTTACGGCTCCGCGCGATCTGGCGACCGCG |
| | 214138 | ACCGTTCTACAAAAAGTACGCGGTTGTACTCGTATGGTACTTCCACAGTT |
| | 214139 | CGTTTCGCTCGCCGCTACTCAGGGAATCGCATTTGCTTTCTCTTCCTCCG |
| | 214140 | GCTACTTGGGACAACACGATCGGAAGACGGCTCACGTCCAGGTACGGGGC |
| | 214141 | AAGGTCCCCCTCTTTGGTCTTGCGACGTTATGCGGTATTAGCTACCGTTT |
| | 214142 | GTTCTGAACCCAGCTCGCGTACCACTTTAATCGGCGAACAGCCGAACCCT |
| | 214143 | TGATTCAAAGCCTCCGGCCTATCCTACACATCAATCACCCAAATTCAATG |
| | 214144 | GTCTTTTCGTCCCATCGCGGGTAATCGGCATCTTCACCGATACTACAATT |
| | 214145 | CCCCCCCCCCCCTTCCCCCCTCTCCTCCCCCTTCCCCCTTTCGCGCCCCC |
| | 214146 | CAGGTGTCACCCCATATACGTCATCTTTCGATTTAGCATAGAGCTGTGTT |
| | 214147 | CTCCACCAGACTAAAACGAGGCTAGCCCTAAAGCTATTTCGAGGAGAACC |
| | 214148 | TTCCGTCAGCCGGCAGGACTGTCACTTCTCCGTCTCCACGTCACTCCATG |
| | 214149 | CGCTAATTTTTCAACATTAGTCGGTTCGGTCCTCCAGTTAGTGTTACCCA |
| | 214150 | CTTGGCAGTGTGACATCACTAACTTCGCTACTAAACTTCGCTCCCCATCA |
| | 214151 | CCCGTTAAATTTTCGGCGCAGAGTCACTCGACCAGTGAGCTATTACGCAC |
| | 214152 | CCCGGAGTACCTTTTATCCTTTGAGCGATGTCCCTTCCATGCGGAAACAC |
| | 214153 | TTCTCTGCGGCTCCATCGCTGCAGCACCCCTTCTCCCGAAGTTACGGGGT |
| | 214154 | AAGCTACCTACTTCTTTTGCAACCCACTCCCATGGTGTGACGGGCGGTGT |
| | 214155 | GCACAGCCATGTGTTTTTGTTAAACAGTTGCCTGGACCTATTCTCTGCGC |
| | 214156 | GCCAACATCCTGGTTGTCTGTGCAATTCCACATCCTTTTCCACTTAACTA |
| | 214157 | GGTCACCCGGTTTCGGGCCCATTATATGCAACTTAACGCCCTTTTCAAAC |
| | 214158 | TTATAGTTACGGCCGCCGTTCACTGGGGCTTCGATTCAATGCTTGCACAT |
| | 214159 | GTTTATCTGAGATTGGTAATCCGGGATGGACCCCTCAATCAAACAGTGCT |
| | 214160 | CGAAGTTACGGGGTCATTTTGCCGAGTTCCTTGACAATGCTTCTTCCGCC |
| | 214161 | GTCCACACACGCGTGTGTCCCTCATCAGTTCTCACCCTCCATGCCCCCCG |
| | 214162 | CCGGCCCGTCGGGGCCGGGACACACGCTCCCGCAACCCCGGCCACGCAAC |
| | 214163 | CCGGTACATTTTCGGCGCAGGGTCACTCGACTAGTGAGCTATTACGCACT |
| | 214164 | CTCGAACTTCTTGTAAGCACACGGTTTCAGGTTCTCTTTCACTCCCCTTC |
| | 214165 | TTTCAGTTCAGGCGGTTCCCCCCGTATCCCTATGGATTCAGAATACGGTG |
| | 214166 | TCCGTTACATTTTGGGAGGCGACCGCCCCAGTCAAACTGCCTACCTGACA |
| | 214167 | CCGCTCCTTCCATCAAGGTTCCACGTGTCTCGATGTACTCTGGATCCTGC |
| | 214168 | CCACGTGTTACTCACCCGTCCGCCGCTAACATCAGGGAGCAAGCTCCCAT |
| | 214169 | GACTCCGTACTGTCAGGTTCGGCTCAACGGGTGGATTTGCCTGCCCATCT |
| | 214170 | ACGTGTCCGGCGGTACTCTGGATACAGATGGCTGTTCAGGCTTTTCGTGT |
| | 214171 | TGGGCTGTTTCCCTTTGGACAATGAAACTTATCTCCCACTGTCTGACTCC |
| | 214172 | ACATAGCTACCCAGCCATGCCCTTGGCAGAACAACTGGTACACCAGCGGT |
| | 214173 | CAGAGGTCAGTCCAACACGGTCCTCTCGTACTAGTGTCAGAGCCACGCAA |
| | 214174 | GTTTGATAGGGTTCAGTAACTTCTCAGCCCCTAGCCCATTCAGTGCTTTA |
| | 214175 | CGGCACCGGGCAGGCGTCACACCCTATACGTCCACTGTTCGTGTTGGCAG |
| | 214176 | AACCCAATAAATCCGGATAACGCTTGCCCCCTACGTATTACCGCGGCTGC |
| | 214177 | CCATACATCAATTATCTGGCATTCTGAGTTTGATAGGGTTCAGTAACCTC |
| | 214178 | CCTCCGTTACACTTTGGGAGGCGACCGCCCCAGTCAAACTGCCCGCCAAG |
| | 214179 | CTGTTATCCCCGAGGTAGCTTTTATCCGTTAAGCGACGGCTTTTCCACTC |
| | 214180 | TAGCCCATTCAGTGCTTTACCTCCGGTAATCTAAATCAACGCTAGCCCTA |
| | 214181 | TCCACAGCTCCTTACGGTACTGCTTCGTCCCGCATGCAATGCTCCTCTAC |
| | 214182 | CCATCGCGGGTAATCGGCATCTTCACCGATACTACAATTTCACCGAGCTC |
| | 214183 | CTGGACCTATTCTCTGCGCCCAACTCTCGTTGGGACCCTTTATCCCGAAG |
| | 214184 | CTTTTACCTTTACACTCTACGATTGATTTCCAACCAATCTGAGCCAACCT |
| | 214185 | TTATAGTTACGGCCGCCGTTTACCGGGGCTTCAATTCAAAGCTTCATATT |
| | 214186 | GCCATTAAGATTCTCACTTAATTCTCGCTACTTATTCCGGCATTCTCACT |
| | 214187 | GGCCGATCACCCTCTCAGGTCGGCTACGCATCGTCGCCTTGGTGAGCCGT |
| | 214188 | CTTCTCCCGCTGGCCTTAGAATCTTCTTCCTATCTACCTGTGTCGGTTTG |
| | 214189 | TTCCTTCACCCGAGTTCTCTCAAGCGCCTTGGTATTCTCTACCTGACCAC |
| | 214190 | GCTAGTCCTAAAACTATTTCGGGGAGAACCAGCTATCTCCGGGTTCGATT |
| | 214191 | CCTCCGGCCGGTTTCACGGCCGCAAGTTAGAATTCCAGCACTACAAGAGT |
| | 214192 | TGTTCGTCCCGTCCTTCATCGGCTCCTAGTGCCAAGGCATCCACCGTGCG |
| | 214193 | GCCAGGCCTTCAAGCCTGTTCCCCTGGCTAGCCGCTTTATGACTCCCGCC |
| | 214194 | CTTTCTTTTCCTCCGGCTACTTAGATGTTTCAGTTCACCGGGTTCCCTTC |
| | 214195 | ATGATTCTCACATAATTCTCGCTACTCATTCCGGCATTCTCACTCGTATG |
| | 214196 | CGGGCACGGACCTTAGCACCCATGCCCTTACTGCCGGACTGCAGACCGTG |
| | 214197 | GTGAGTTTCCTCATTCAGAGATCTCCGGATCAATGCTTATTTGCAGCTCC |
| | 214198 | TAAATGCAGTCCGAACCCCGGAGTGCACGCACTCCGGTTTGGGCTCTTTC |
| | 214199 | GCCCAAGGGTAGATCACTTGGTTTCGCGTCTACTCCTTCCGACTATACGC |
| | 214200 | AGCTTAGCGGATTTTCTCGGGAGTCTGATTACCGGCGCTATTGGATTCCA |
| | 214201 | CTCGCAGTCAAGCTCCCTTCTGCCTTTGCACTCTCCGAATGATTTCCAAC |
| | 214202 | GTCTAGTCCCACGTACTTGTGCGCCCTGTTCAGACTCGCTTTCGCTCCGC |
| | 214203 | TTCTCCGCTATCCACACCTCATCGCCACCCTTTTCAACGGATGTGCGTTC |
| | 214204 | GCCGGCTCCCATTCCGTGTCACCCCTGCGCTCACCTACCACGGCTACGCT |
| | 214205 | TCCCGGGGTCCTTTTCACCTTTCCTTCACAGTACTATGCGCTATCGGTCA |
| | 214206 | CCAACATCCTGGTTGTCTGTGCAATTCCACATCCTTTTCCACTTAAATCC |
| | 214207 | GCTGGCGCCGCGGCTTCGAAGCCTCCCGCCTATGCTACACAATCCGCACC |
| | 214208 | ACGCCCAATAATTCCGGACAACGCTTGCCACCTACGTATTACCGCGGCTG |
| | 214209 | CCCTACCAGGTATCACATGCACACGGTTTAGCCTCATCCACGTTCGTTCG |
| | 214210 | AGCACCGGGCAGGTGTCAGGCTGTATACGTGATCTTTCAATTTGGCACAG |
| | 214211 | CTCCCCATCATGCCTCAACCTTCACGCCCAGCGGATTTACCTACCAGACA |
| | 214212 | CTTCAACTTAACCTCGCACGTAAACGTAACTCGCCGGTTCATTCTACAAA |
| | 214213 | AGAGTAGCCATAACACAAGGGTAGTATCCCAACAACGCCTCAGTCGAAAC |
| | 214214 | GCTCGCGTACCACTTTAAATGGCGAACAGCCATACCCTTGGGACCTACTT |
| | 214215 | CATAGACCTGTGTTTTTGCTAAACAGTTGCTTGAGCCTATTCTCTGCGGC |
| | 214216 | ACACACAACCCCTACCAAGTATCACATGCACACGGTTTAGCCTCATCCAC |
| | 214217 | TCTACGACCACGTACTCATGCGCCCTATTCAGACTCGCTTTCGCTGCGGC |
| | 214218 | CATTCGGATATCTCTGGATCAAGGCTTACTTACAGCTCCCCAAAGCATGT |
| | 214219 | GCTCTCCTACCACTGTTCGAAGAACAGTCCGCAGCTTCGGTGATACGTTT |
| | 214220 | TCTTTTCGTCCCATCGCGGGTAATCGGCATCTTCACCGATACTACAATTT |
| | 214221 | TGTACCCCCCATTGTAACACGTGTGTAGCCCCGGACGTAAGGGCCGTGCT |
| | 214222 | TCCCCGGAGTACCTTTTATCCTTTGAGCGATGTCCCTTCCATACGGAAAC |
| | 214223 | CGTTGAGCGATGGCCCTTCCTTTCGGTACCACCGGATCACTAAGCCCGAC |
| | 214224 | TTCAAGGGGTCTTACTCGTTATACGATGGGATATCTAATCTTGGAGTCGG |
| | 214225 | CCTCCTGATGTCCGACCAGGATTAGCCAACCTTCGTGCTCCTCCGTTACT |
| | 214226 | ACCTTGGTCTTACGGCGGGAGGGAATCTCACCCTCCTTATCGTTACTTAT |
| | 214227 | CGTGCCCCGCCCTACTCAGGATACTGCTAGCCACGATCAACTTTTAGGTA |
| | 214228 | CACCCTCAGTTCATCCGGAAGCTTTTCAACGCTTATCGGTTCGGTCCTCC |
| | 214229 | TCTACCTCCATGAGACTAATACGAGGCTAGCCCTAAAGCTATTTCGAGGA |
| | 214230 | TACCTGTGTCGGTTTGCGGTACGGGCACCTTAGCATACACTAGAACTTTT |
| | 214231 | AGCGGTTCCACAGCTTGTAAACATATGGTTTCAGGTTCTCTTTCACTCCC |
| | 214232 | TTATAGTTACGGCCGCCGTTCACTGGGGCTTCGGGTCAAAGCTTGCACTC |
| | 214233 | TTATAGTTACGGCCGCCGTTTACTGGGGCTTCGGTTCGATGCTTCGATTG |
| | 214234 | GCCTTACGGGGTGGTCCCCGCTCATTCCCACAAGGTTTCTCGTGTCTCGT |
| | 214235 | CCGGAGTTTTTCACACTGAGCCATGCAGCTCTGTGCGCTTATGCGGTATT |
| | 214236 | CTTCTCCCGTTGGCCTTAGAATCTTCTTCCTACCTACCTGTGTCGGTTTG |
| | 214237 | TGCCGCTTTAATGGGCGAACAGCCCAACCCTTGGGACCGACTACAGCCCC |
| | 214238 | GGAGTTCTTCGTGATATCTAAGCATTTCACCGCTACACCACGAATTCCGC |
| | 214239 | AGTGATGGGCAGGTTGGATACGCGTTACTCACCCGTGCGCCGGTCGACGC |
| | 214240 | TCACGGTACTCGTACGCTATCGGTCAGACAGGTATACTCAGGCTTACCCG |
| | 214241 | ACGCATTCGGAGTTTGTCAAGACTTGATAGGCGGTGAAGCCCTCGCATCT |
| | 214242 | CATCATCTGTATGGCATTCGGAGTTTGATATCCCTTAGTAAGCTTTGACG |
| | 214243 | TTCTCCGCTATCCACACCTCATCGCCACCCTTTTCAACGGATGTGCGTTC |
| | 214244 | AAGCACTTTGGTTTGGGCTGTTCCCCGTTCGCTCGCCGCTACTTAGGGAA |
| | 214245 | CACTTATGCCCGATTATTATCCACGCCAAACTCCTCGACTAGTGAGCTGT |
| | 214246 | CTTAGGACCCGACTCACCCAGGGCAGACAAACTTGACCCTGGAACCCTTG |
| | 214247 | CTCATCAGTTCTCACCCCCAATGTCCCCCGGATTTACCTGAGGGACGGGC |
| | 214248 | CCCATGGTGCACGCACCATGGTTTGGGCTCTTCCGCGTTCGCTCGCCGCT |
| | 214249 | GCTAGTCCTAAAACTATTTCGGGGAGAACCAGCTATCTCCGGGTTCGATT |
| | 214250 | ACCCCATCAATTAACCTTCCGGCACCGGGCAGGCGTCACACCGTATACGT |
| | 214251 | CATTCCGGCATTCTCACTCGAATACAATCCACCGCTGCTTCCGCTACGAC |
| | 214252 | GTTTCAGTTCGCCGGGTACCTCTCTTGCAGGCCATGTATTCACCTGCAGA |
| | 214253 | ACCTGAGGCTACTCGCCTCGACTACCTGTGTCGGTTTGCGGTACGGGTAG |
| | 214254 | AAGGCTAGCCCTAAAGCTATTTCGAGGAGAACCAGCTATCTCCGGGTTCG |
| | 214255 | ATTATTATTTTCTCCTCCTACGGGTACTGAGATGTTTCACTTCCCCGCGT |
| | 214256 | GCTTGCGCTAACCTCTCCTCTTAACCTTCCAGCACCGGGCAGGCGTCAGC |
| | 214257 | CAGAGGTCTGTCCAACACGGTCCTCTCGTACTAGTGTCAGAGCCACGCAA |
| | 214258 | ATCCTCTCAGACCAGTTACGGATCGTCGCCTTGGTAGGCCTTTACCCCAC |
| | 214259 | TCACGCAGAATTCCTCGTGCTCCGCGCTACTCAGGATACCACTAGGCTTC |
| | 214260 | CGCGTCTTCGGTGGCGTGCTTGAGCCCCGCTACATTGTCGGCGCGGAACC |
| | 214261 | TACTTATGCCCGATTATTATCCACGCCAAACTCCTCGACTAGTGAGCTGT |
| | 214262 | ACCGTAGTGCCTCGTCATCACGCCTCAGCCTTGATTTTCCGGATTTGCCT |
| | 214263 | AGCTGACGCCTGTATTTCCCAGTCTCCCACCTATCCTGTACATGAAATAC |
| | 214264 | GGCGTTGCTGATCCGCGATTACTAGCGACTCCGCCTTCACGGAGCCGGGT |
| | 214265 | GGGTGCCGCATGGGTTAAGCTTAGCGGATTTTCTCGGGAGTATGGTTACC |
| | 214266 | TCTTCAGCCCCAGGATGCGATGAGCCGACATCGAGGTGCCAAACTTCCTC |
| | 214267 | CGCCGGCACCGGATCACTATCTCCGACTTTCGTCCCTGCTCGATCCGTCG |
| | 214268 | CACACTATCCGTCTCCGTCACTCCTTCGCTCCATATACGGGTGCAGGAAT |
| | 214269 | ACTGTCAGGTTCGACTCTTCCTGCGGATTTGCCTGCAGGAATCAACATCT |
| | 214270 | TCTTTCGGCGAGGGGGTTTCCCACCCCCTTTATCGTTACTTATACCTACA |
| | 214271 | CTTTTCAGTGCTCTACAGGACACATCCATCACCTGAGGCTGTACCTCAAT |
| | 214272 | ATGACCCTCCCCGGTTGAGCCGGGGGCTTTCACATCAGACTTAAGAAACC |
| | 214273 | TTTCACAACTGACTTAAATATCCATCTACGCTCCCTTTAAACCCAATAAA |
| | 214274 | CTACTTATTTTCGGTCCCTTACGCCCGGGTCAACCAACGCCCGGGTCCAG |
| | 214275 | GTATTTAGGCTTACCGGGTGGTCCCGGCAGATTCACAGCAGATTCCACGA |
| | 214276 | CTTCAACCTGGACATGGATAGGTCACCCGGTTTCGGGTCTGCACACACTG |
| | 214277 | TCCGGAAGCCACGCCTCAAGGGCACAACCTCCAAGTCGACATCGTTTACG |
| | 214278 | GGTCACCCGGTTTCGGGCCCATTGTATGCAACTTAACGCCCTTTTCAAAC |
| | 214279 | GGCTACACATTTTAAAATGCTTAACCTTGCCGGAAAAAGTAACTCGTAGG |
| | 214280 | CAAATTTCCTGCGCCCGCGACGGATAGGGACCGAACTGTCTCACGACGTT |
| | 214281 | GCCAGGGTAGTATCCCACCGATGCCTCCACCGAAGCTGGCGCTCCGGTTT |
| | 214282 | TTCACTGAAGGGTAACACCCCATAACAGGTGCCAGGTTTCCCCATTCGGA |
| | 214283 | TCCAGCTAATCAGACGCGGGTCCATCTTATACCACCGGAGTTTTTCACAC |
| | 214284 | CTTTATGAATATGCTTAGCGGATTTTCTTGGGAGCCTGATTACGTCCATT |
| | 214285 | CATCAGGTAGTATTCAGGCTTACCAGGTGGTCCTGGCAGATTCACACGAA |
| | 214286 | CATGCACCACGGATTTGCCTATGATGCGCGCTGCGTGCTTGACCACGGAA |
| | 214287 | GACAGCCTGGCCATCATTACGCCATTCGTGCAGGTCGGAACTTACCCGAC |
| | 214288 | TCACTGCTTTAAGCAGCTCCGACCGCTTGTAGGCGCACGGTTTCAGGAAC |
| | 214289 | GCTCCCAACACCACGCGGCGATACCAACCCGAAGGAAGGAACCACCACGA |
| | 214290 | GACTTCCCATTCCATTCCACTAAACCTTTACAATACCGTTTTCTGTCCGA |
| | 214291 | ACTTAACGACCCGTCTGCGCTCCCTTTAAACCCAATAAATCCGGATAACG |
| | 214292 | GGGGTGGGTTTCATACTTAGATGCTTTCAGCAGTTATCCGCTCCGCACTT |
| | 214293 | GAAATCCTCGGATCAAAGCCCTGCTGGCGGCTCCCCGAGGCATATCGCAG |
| | 214294 | CTTTCATGGCCCCTACTGATCATCGCCTTGGTAGGCCATTACCCTACCAA |
| | 214295 | CTGTTATCCCCAGGGTAACTTTTATCCGTTGAGCGATGGCATTTCCACTC |
| | 214296 | CCTACCCTCAGCTCATCCAGAAGCTTTTCAACGCTTATTGGTGCGGTCCT |
| | 214297 | ACCAAGAAGGTGCTCCGACCGCTTGTAGGCACATGGTTTCAGGAACTATT |
| | 214298 | CTTCTCCCGTTGGCCTTAGAATCTTCTTCCTACCTACCTGTGTCGGTTTG |
| | 214299 | CCTGGCCAAGGGTAGATCACTTGGTTTCGCGTCTGCCACTGCCGACTATA |
| | 214300 | GGGGGTCTCCCTTATGCCGAAGGCACGGGAGCAATTTGCCGAGTTCCTTG |
| | 214301 | CATGGTTTAGCCCCGTTACATCTTCCGCGCAGGCCGACTCGACCAGTGAG |
| | 214302 | ATCCGCCGCCTTTTCAACGGAGGTCGGTTCGGTCCTCCATGGAATTTTAC |
| | 214303 | CCAAAGTCAATGCTAAGCTGTAGTAAAGGTTCACGGGGTCTTTTCGTCCC |
| | 214304 | AAAGTTCGGTGGTTACGGAATTTCTACCGTATGTGCATCGACTACGCCGT |
| | 214305 | CAGGTGTCAGCCCCTATACTTCATCTTTCGATTTAGCAGAGACCTGTGTT |
| | 214306 | ACTTAAAGCCAGCGCCCCTTCTCCCGAAGTTACGGGGCCATTTTGCCGAG |
| | 214307 | ACTTAGATGCTTTCAGCACTTATCCGATCCAGACTTAGATACCCGGCAAT |
| | 214308 | CTACAGGATTTAGTTTAGCGGATTTTCTTGGCAGCATGATTACATGCACT |
| | 214309 | CCTTAACCTTCCGGCACTGGGCAGGTGTCAGCCCGTATACGTCGTATCTC |
| | 214310 | TGAGCCAACATCCTAGTTGTCTTCGAAATCCCACATCCTTTTCCACTTAA |
| | 214311 | CAGGATGTGACGAGCCGACATCGAGGTGCCAAACCCCTCCGTCGATATGA |
| | 214312 | GGTTTTGCCGGTCCATGGTCGGTACGGGAATATCCACCCGTTCATCCATT |
| | 214313 | CTTTACGCTATCGGTCATTGGGTAGTATTTAGGCTTGGAGGGTGGTCCCC |
| | 214314 | GCATGGATTAAGTTTAGCGGATTTTCTAGGAAGTATGATTACCTACGCTA |
| | 214315 | ACTGTCCATCCTCTGGTTTCACAGAGCTATGTTAGAATTTCAGTAACCGA |
| | 214316 | ACCTCGCGGTACGCCTTCGACGCCGACTGGAATGCTCCCCTACCGATCAT |
| | 214317 | CTCTTGCGATGAGCTCTCCTCTTAACCTTCCAGCACCGGGCAGGTGTCAG |
| | 214318 | AGCTGACGCCTTGGCTTCCCAGTCTCCCACCTATCCTGTACATGTAATAC |
| | 214319 | GAATGAATGGCTGCTTCCAAGCCAACATCCTAGCTGTCACTGGGACCAGA |
| | 214320 | TGAGCCAACATCCTGGTTGTCTACGTATCTTCACATCGTTTTCCACTTAA |
| | 214321 | TGAGGGCACCTTTAGAAGCCTCCGTTACGCTTTTGGAGGCGACCACCCCA |
| | 214322 | TTAAATCGACCGAAGTTTCAATAAAGTAATTCCCGTTCGACTTGCATGTG |
| | 214323 | AGTCGGGTTGCAGACTCCAATCCGAACTGAGAGAGGCTTTAGGGATTAGC |
| | 214324 | CCTGTGTCGGTTTACGGTACGGGTATGGTATGAACAATAGCGGCTTTTCT |
| | 214325 | CTCCCGGATTCCGACGGAATTTCACGTGTTCCGCCGTACTCAGGATCCAC |
| | 214326 | AAACATTAAAGGGTGGTATTTCAAGGTCGGCTCCATGCAGACTGGCGTCC |
| | 214327 | CCTGAGTATATTCAACCCGACTACGTGTGTCCGTTTACGGTACGGGTACC |
| | 214328 | ACCACGAATTCCGCCTGCCTCAACTGCACTCAAGATATCCAGTATCAACT |
| | 214329 | AGTGAGCTATTACGCACTCTTTTAATGAGTGGCTGCTTCTAAGCCAACAT |
| | 214330 | GGCTCACGCCCCGCCTTCAACGCCGAGTGGAATGCTCCCCTACCGATGAT |
| | 214331 | AGGGCACCTTTAGAAGCCTCCGTTACACTTTTGGAGGCGACCACCCCAGT |
| | 214332 | CTCTGCCATCGCCATCGCCGTTCGGCTTAGACTTAGGACCCGACTGACCC |
| | 214333 | GCCGAGTTCCTTAACAAGGGTTCTCCCGCTCGTCTTAGGATTCTCTCCTC |
| | 214334 | CTCCCCCCCCCCCCTTCCCCTCCGCGGCCACCTTTCCCCCCCCCTCCCCA |
| | 214335 | CCCATATACACGGGTTAGAATCCAAACAAATGAAGGGTCGTATTTCAACA |
| | 214336 | CCCGCATCAGCGGGTTAGAACTCAAATAATCAAAGGGCCGTATTTCAACA |
| | 214337 | CTTCACAGTACTATACGCTATCGGTCACTGGGTAGTATTTAGGGTTGGAG |
| | 214338 | CATTCCCACTTAATACCACCGGATCACTAAGCCCTACTTTCGTACCTGCT |
| | 214339 | CTTCCGTCGCCCCGCGGTGGTTTCACTGCTCCGTCTCCACGTCGCCCCAT |
| | 214340 | GCGGGTAACCTGCATCTTCACAGGTACTAAAATTTCACCGAGTCTCTCGT |
| | 214341 | AAAAGTACGCGGTTGAGCTAATAATGCTCTTCCACAGCTTGTAAACACAG |
| | 214342 | CGGTACGGGAATATCAACCCGTTCATCCATTCGACTACGCCTGTCGGCCT |
| | 214343 | CCTCATCTACCTGTGTCGGTTTGCGGTACGGGCGCCTTAGTATACCTCAT |
| | 214344 | GTAGTATTTAGCCTTGGAGGGTGGTCCCTCCTGCTTCCCACAGGGTTTCA |
| | 214345 | TTCCGTCAGGTGGCGGCACTTACGTTCCTTCGTCTCTCCATCGAGGTATA |
| | 214346 | CTTCAAAGTCTCCGGCCTATCCTACACATCAATTACCCAAATTCAATGTT |
| | 214347 | CTCTCAGGGCTCTTACTAACTGAACGTTATGGGAAATCTCATCTTGAGGG |
| | 214348 | AAGTCCTCGAGCGATTAGTATTGGTCCGCTTCACGTCTCACAACGCTTCC |
| | 214349 | ACGCCTTTCGTGCAGGTCGGAACTTACCCGACAAGGAATTTCGCTACCTT |
| | 214350 | CCTGATCGACTTGTATGTCTCCCAGTCAAGCGCCCTTATGCCATTACACT |
| | 214351 | CGTTTTCCACTTAGCATGTATTAGGGACCTTAGCTGTGGGTCTGGGCTGT |
| | 214352 | TAGTCAAGTATCGTCTCTCTTCTTCCTTGCTGATAGACCTTTACATACCG |
| | 214353 | GACACATGGTTTTCTGCAACTGCCGGCCGGCCCGTCGGAGCCGGCGCACG |
| | 214354 | TTTCTCGTGTCTCGTGGTACTCTGGATCCCGCCTTGCCGCTCCCGGTTTC |
| | 214355 | CTAATGAGATGTTTCAGTTCACAGCGTTTACCTCCAACTAGACTATGAAT |
| | 214356 | ATCCTTTCCCACTTAGCACGCGCTTGGGGACCTTAGACGACGATCTGGGC |
| | 214357 | GTTTCACGTGTCTGGCCGTACTCTGGAACTCGCTCAGCTCTTGTCGTTTT |
| | 214358 | ATGGTTATAGTTACCACCGCCGTTTACCGGGGCTTGAATTCACCGCTTCG |
| | 214359 | CCGCACGGAATGGCCGTCTCGTCTCGGGGGGGGCTTCCCGCTTAGATGCT |
| | 214360 | TGCTCGACTTGTCTGTCTCGCAGTCAAGCTCCCTTATACCTTTACACTCT |
| | 214361 | ATGCATTGCCAGAAGCTTTTCCTGGAAGCCGTCATCATGTGCTTCGCTAC |
| | 214362 | TCTTGCGGCGAGCAGGTTTCTCACCTGCTTTATCGTTACTTATACCTACA |
| | 214363 | CGCGCACGCAACCCCCGACGGGTATCACGCGCACGCGGTTTGGTCTGATC |
| | 214364 | CGCTTTATCGTTACTTATGTCAGCATTCGCACTTCTGATACCTCCAGCAT |
| | 214365 | GACAGTGCCCAAATCATTACGCCTTTCGTGCGGGTCGGAACTTACCCGAC |
| | 214366 | TCCCATCTATCCTGTGCATGCAACACCGAAACCCAATATTAGGCTACAGT |
| | 214367 | CCCGGGTCATGCCCTTTCAGAGTGTCCCTCTGCTTAAAACTTTCGGTGGT |
| | 214368 | GGGATCCCATTCCCGGCTTCCGCTCTCTGCACGTGTCCCCACAGTTCTGT |
| | 214369 | CACCTCGCCATACACGCCGCACGGATTTGCCTATGCGACTGGCTGCGTGC |
| | 214370 | TCGCTCCTCAGCGTCAGTTACAGACCAGAGAGTCGCCTTCGCCACTGGTG |
| | 214371 | TATCGAACCATAACGGCTCCCATCATCACACCTCGCCATGCATGCCATGC |
| | 214372 | TTCACCGGGGCTTCAATTCGGAGCTTGCACCCCTCCTCTTGACCTTCCGG |
| | 214373 | CTGCAGGATTAAGTTTAGCGGATTTTCTCGGCAGCATGCTTACGCGCACT |
| | 214374 | TCTCCTACCATACCTATAAAGGTATCCACAGCTTCGGTAATATGTTTTAG |
| | 214375 | GGGCGCGTCATGCCCTCACGTCGAGGCTTTTCTCGGCAGCATAGGATCAC |
| | 214376 | CTCCGACGGATTGTAGGCGCACGGTTTCAGGAACTCTTTCACTCCCCTCC |
| | 214377 | CACTCGACTAGTGAGCTATTACGCACTCTTTGAATGAATAGCTGCTTCTA |
| | 214378 | ACTCCCCTCGCCGGGGTTCTTTTCGCCTTTCCCTCACGGTACTGGTTCAC |
| | 214379 | CCCTCCCGGGGTTCTTTTCACCTTTCCCTCACGGTACTATGCGCTATCGG |
| | 214380 | CTGGTCCTCTCGTACTAGGAGCAGATCCTCTCAAATTTCCTTCGCCCGCG |
| | 214381 | ACTTTCGTTACTGCTCGGGCCGTCACCCTCGCAGTTAGGCTAGCTTTTGC |
| | 214382 | TGTAATAGCCACGTAATTTAAAACTGAAATTGAGAGAGACTTACCCAGAG |
| | 214383 | GGTGGTCTACCGGGAGACTTACCCTCATGTGAGGTGGGAATACTCATCTT |
| | 214384 | TGGCGGTCTGGGCTGTTTCCCTTTCGACTACGGATCTTATCACTCGCAGT |
| | 214385 | TCTCCACATCACTCTTATAGGTAGTACAGGAATATTAACCTGTTCTGCCA |
| | 214386 | CCATTCTGAGGGTACCTTTGGGCGCCTCCGTTACTCTTTCGGAGGCGACC |
| | 214387 | GATGGCAGGACTGTCACTTCTCCGTCTCCACATCGCTCCATAAAGTAGTA |
| | 214388 | TCGGCGCAGAGTCACTCGACCAGTGAGCTATTACGCACTCTTTAAATGGT |
| | 214389 | CGCGGCATGGCTGCATCAGGCTTGCGCCCATTGTGCAGTATTCCCCACTG |
| | 214390 | CGGACATCCTTAATGACATTCGCAGTTTGATTGTATTCAGTACCCCGGGA |
| | 214391 | TACCGGCATTCTCACTTCTAAGCGCTCCACCAGTCCTTCCGGTCTGGCTT |
| | 214392 | TTCGGGCCTCCATTCAGTGTTACCTGAACTTCACCCTGGACATGGGTAGA |
| | 214393 | CGGAGGCGACCGCCCCAGTCAAACTCCCCGCCTGGCATTGTCCCACCGCC |
| | 214394 | ACCTTTTAGGAGGCGACCGCCCCAGTCAAACTGCCCGTCAGACACTGTCT |
| | 214395 | ACAGCCCAGCCTTCCGTTGTGCGTACTTCACTACACAACAGCCTCACTGC |
| | 214396 | TCATACCACCGGAGTTTTTACCCCTGCACCATGCGGTGCTGTGGTCTTAT |
| | 214397 | CACTCACCCGAAGGCTTGCTCCCAAACAAAAGAGGTTTACAACCCGAAGG |
| | 214398 | CGTCAATTCATTTGAGTTTTAACCTTGCGGCCGTACTCCCCAGGCGGTCG |
| | 214399 | ACTTTCGTTCCTGCTCGACTTGTCAGTCTCGCAGTCAGGCTGGCTTGTGC |
| | 214400 | CCACCAGGGAGGCTCCGACGGTTTGTGGGCGCACGGTTTCAGGAACTGTT |
| | 214401 | ACTGGCGTGCACGTCTCTTTGTCTCCCACCTATCCTGTACATGTATGACC |
| | 214402 | TGATAGCGTGAGGTCCGAAGATCCCCCACTTTCTCCCTCAGGACGTATGC |
| | 214403 | AAATCTTTAATCTCTTTCAGATGTCTTCTAGAGACGTCATTGGGTATTAG |
| | 214404 | CACCGGGGCCCCAAGACCCACACACACCAACAAACCCGAAGGCTTAGTGG |
| | 214405 | TACTTTTCCAATTTTTTTTTTTTTTTTTTTTTTTTTTTTCTTCCAATAAA |
| | 214406 | CTCTGCCTATCCTTCTGTGTCACTGCATCCGGTTGCTCGGCGGTATCGGA |
| | 214407 | ATGCCTGGCAGTTCCCTACTCTCGCATGGGGAGACCCCACACTACCATCG |
| | 214408 | AACATCCTGGTTGTCTAAGCAACTCCACATCCTTTTCCACTTAACGTATA |
| | 214409 | CTCCGGCCGGGCCCGCCAGGACCCGGACACACGCTCCCTCAACACCACGC |
| | 214410 | TTCTCTGCGGCTCTTTCGAGCACTCCTTATTCCGAAGTTACGGAGTCAAT |
| | 214411 | GGCACAGCCCTGTGTTTTTGTTAAACAGTTGCCTGGACCGATTCTCTGCG |
| | 214412 | TGCTCCCCACGCTTTCGAGCCTCAACGTCAGTTACTGTCCAGTAAGCCGC |
| | 214413 | ATGCGTCCCACGGATTTGCCTATGGGACGGGCTGCGTGCTTGACCACGGA |
| | 214414 | CCCAGACAACCATCGCTGGGGTTGAGCTACCTCCCTGCGTCCCTCCGCAG |
| | 214415 | ACGCCGTTAGGCCTCACCTTAGCTCCCGACTGACCTGGAGCGGACGAACC |
| | 214416 | GCCTTTAGCCTTAACCTTGCCAGCCGGCGTAACTCGCCGGACCGTTCTAC |
| | 214417 | TGGCCGTTCAACCTCTCAGTCCGGCTACTGATCGTCGCCATGGTGAGCCG |
| | 214418 | CGCTTTCGCTCGCCACTACTCACGGAGTATCCCTTCCTGCAGGTACTGAG |
| | 214419 | AGGACCCGACTCACCCGGGGACGACGAACGTGGCCCCGGAACCCTTGGTC |
| | 214420 | CATTGCGGAAGATTCCCCACTGCTGCCTCCCGTAGGAGTCTGGACCGTGT |
| | 214421 | GCATGTATTAGGCACGCCGCCAGCGTTCGTCCTGAGCCAGGATCAAACTC |
| | 214422 | CCCGTTACCCATCATCGCCATGGTAGGCCTTTACCCTACCATCTAGCTAA |
| | 214423 | GCCCTCACCCGATTAGTAACAGTCAGCTCCATGTGTTGCCACACTTCCAC |
| | 214424 | ACCCCAAGTCATCCCCCGGTTTTCAACCCAGGTGGGTTCGGTCCTCCACG |
| | 214425 | CGCCTTAGGACCCGACTAACCCAGGGCGGATAAACCTAGCCCTGGAACCC |
| | 214426 | TTCCGTCTTGCCGCGGGTACACTGCATCTTCACAGCGAGTTCAATTTCAC |
| | 214427 | GTACGGGTAACACAGAAATATGCTTAGCGGGTTTTCTTGGGAGCCGGTTT |
| | 214428 | AAGCTCCATGGGGTCTTTCCGTCTTGTCGCGGGTAACCGGCATCTTCACC |
| | 214429 | AACTTTATTCCCTTATAGAAGCAGTTTACAACCCATAGGGCCGTCTTCGT |
| | 214430 | GGGCGGGATTCGCACCCGCCTCTCGCTACTCATGTCTGCATTCTCACTCC |
| | 214431 | ATACTATCAGGTTCGGATCTCATGGTGGATTTGCCTGCCATGATCGACTC |
| | 214432 | ACGCCGTCGGGCATATAAAGCCCTCCGACAGTTTGTAAACACAGGGTTTC |
| | 214433 | GCCTATCGACCACGTGTTCTGCATGGGGTCTTCAGCGGCTCGGGGCCGCA |
| | 214434 | GGATAAGGGTTGCGCTCGTTGCGGGACTTAACCCAACATTTCACAACACG |
| | 214435 | GCCCCCGAGCCTTGGCAGTGCTCTACACGGCGTGAGGTTCATCCGAGGCT |
| | 214436 | TTCCTTAACCAAGAATCTCTCAACGCCTTAGTATGTTCTACCCGACCACG |
| | 214437 | TTTCCCTGCGGCTCCGGGACTTTATCCCTTAACCTTGCCAGTATGCACAA |
| | 214438 | TACTGTCAGGTTCGACTCTTGCACCGGATTTGCCTGGCACAATCAACATC |
| | 214439 | GCCTTCCCATGCCATTCTGCTAGATACCTTCCATACCGTGCGCTGTCCGA |
| | 214440 | ATGAGCCGACATCGAGGTGCCAAACACCGCCGTCGATATGAACTCTTGGG |
| | 214441 | TTCGGCTCAAAGTCCGGATTTGCCTGGACCTCTCATCACCTACACTCTTC |
| | 214442 | ACGCATTTCACCGCTACACGTGGAATTCCACTCTCCTCTTCTGCACTCAA |
| | 214443 | TTTCCGTTTCGCCTACGGGGCTCTCACCCTCTCTGGCCGGTCTTTCCAGA |
| | 214444 | GCCCCGGACAACCATCGCCGGGGATGAGCTACCTCCCTGCGTCCCTCCGC |
| | 214445 | TGTCGCGGGTAACCGGCATCTTCACCGGTACTACAATTTCGCCGGGCGGG |
| | 214446 | AAGCCCTCGATCTATTAGTACACACTTGCTGAATGGATCGCTCCACTTAC |
| | 214447 | CCTTGGCAACAGTTCTCTCGCTCACCTCGGGATACTCTCCCTGCCCACCT |
| | 214448 | TCTCCGCCAAAGCCAAAGCCTTGGTTTCCCAGAGTCCCATCTATCCTGTG |
| | 214449 | AGGAGTATTCAGGCTTACCAGGTGGTCCTGGCAGATTCACACGAGATTTC |
| | 214450 | CAGGATGTGACGAGCCGACATCGAGGTGCCAAACCACTCCGTCGATATGA |
| | 214451 | CAACCTGTTGTCCATCGGCTACGCTTTTCAGCCTCACCTTAGGTCCCGAC |
| | 214452 | TCAGATGGCGGCACTGCCACGACTCCGTCTCCACGTCACTCCCCAAGGTA |
| | 214453 | CTACGGGGCCATCACCCTCTGCGGCCCGGCATTCAATCCGGTTCGCCTCA |
| | 214454 | CCAGGTCATAAGGGGCATGATGATTTGACGTCATCCCCACCTTCCTCCGG |
| | 214455 | CCTTTAATCATGTGAACATGCGGACTCATGATGCCATCTTGTATTAATCT |
| | 214456 | TTTTCACACCTGACTTAAGATCCCGCCTTAAGCTTCCCTTTACACCCAGT |
| | 214457 | CCTACCCTCAGCTCATCCAGAAGCTTTTCAACGCTTATTGGTGCGGTCCT |
| | 214458 | GTCACACTGAGTATTTAGGCTTACCGGGTGGTCCCGGCAGATTCACAGCA |
| | 214459 | CCAGGATAACTTACGTACACCATTCGACGCCGTGAGTATGCTCCCCTACC |
| | 214460 | AGAGAACCAGCTATCTCCAAGTTCGTTTGGAATTTCTCCGCTACCCACAA |
| | 214461 | CCCGAAGTTACGGGGTAATTTTGCCGAGTTCCTTAACAACCCTTCTCCCG |
| | 214462 | GGCTCACGCCCCACCTTCGACGCGGAGTGGAATGCTCCCCTACCGATGTT |
| | 214463 | GTATCTAATCCTGTTTGCTCCCCACGCTTTCGCACTGAGCGTCAGTCTTC |
| | 214464 | CGCGAGTCCATCCTGAAGCGAATAAATCCTTTTCCCTCAGCACCATGCGG |
| | 214465 | TTATCGCAGCTTATCACGTCTTTCTTCGGCTCTTAGTGCCAAGGCATCCA |
| | 214466 | CGGCAAAGATTCTCACTTTGCTCTCGCTACTCATGCCGGCATTCTCTCTC |
| | 214467 | CCGGCAGACCGATCAAGAAAAAACCCACAACCCCGCACGCGCAACCCCTG |
| | 214468 | GGGCTGTTTCCCTTTTGACTATGAGACTTATCTCACATAGTCTGACTGCT |
| | 214469 | CCCCACTGCTGCCTCCCGTAGGAGTCTGGACCGTGTCTCAGTTCCAGTGT |
| | 214470 | TTGTGACTATTCTCTGCGGCCTGCTCTCGCAGGCACCCCTTATCCCGAAG |
| | 214471 | TTACCTCCACTTCAACCTGGACATGGGTAGGTCACCCGGTTTCGGGTCGA |
| | 214472 | TCGCAAGGTTATCCCCAAGTGAAGGGCAGGTTGGATACGCGTTACTCACC |
| | 214473 | CGCGATCGGCAGACCATGCGCGTTCAGGTACGGGGCCCTCACCCTCTGCG |
| | 214474 | GCCTTTCACTCCTACACTCGGCTCATCCAGAAGCTTTTCAACGCTTATTG |
| | 214475 | AGTTTGATAAGGTTCAGTAACCTCTCGGCCCCTAGCCAATTCAGTGCTTT |
| | 214476 | GGCTGCAACACGGTGACGTGAAGCGAATCCCAAAAACCATCTCTCAGTTC |
| | 214477 | CCGGTCTCTCGACTAGTGAGCTGTTACGCACTCTTTGAATGAATGGCTGC |
| | 214478 | GGATCACTAACTCCAACTTTCGTTACTGCTCGAACTGTCGCTCTCGCAGT |
| | 214479 | CTCGCGTACCGCTTTAATGGGCGAACAGCCCAACCCTTGGGACCGACTAC |
| | 214480 | CGGCTACGCCTTTCGGCCTCACCTTAGCTCCCGACTAACTTGGAGCGGAC |
| | 214481 | ACCTTTCCCTCACGGTACTGGTTCACTATCGGTCACTAGGGAGTATTTAG |
| | 214482 | ATACTGTCAGGTTCGACTCTTGCACCGGATTTGCCTGGCGCAATCAGCAT |
| | 214483 | TGTCATGCTCTATGGTCTTTCTTTCCAGAAAGTTCTTCTCCGATGTCTTC |
| | 214484 | ATCACCTTAGGATTCTCTCCTCGCCTACCTGTGTCGGTTTGCGGTACGGG |
| | 214485 | ACGTATTCACCGTGGCATTCTGATCCACGATTACTAGCGATTCCGACTTC |
| | 214486 | TAGAGCATTTTCTTGGAAGCAGGATTACCCACACTATTGGTTTACTCCGA |
| | 214487 | CATTGACCAATATTCCTCACTGCTGCCTCCCGTAGGAGTTTGGGCCGTGT |
| | 214488 | ATCCGCCGCCTTTTCAACGGAGGTCGGTTCGGTCCTCCATGGAATTTTAC |
| | 214489 | CCTGTGTCGGTTTACGGTACGGGCGCATGGCAAACGATAGCGGCTTTTCT |
| | 214490 | GCCCAAGGGTAGATCACTTGGTTTCGCGTCTACTCCTTCCGACTATACGC |
| | 214491 | GGCGGATTTTCCCAAATCCTTCGACTATCAAGTTCTTTGGTAACTCAAAT |
| | 214492 | CTTTCGGGGAGTACGAGCTATCTCCGAGTTTGATTGGCCTTTCACTCCTA |
| | 214493 | CTCTAGTTAGCCTGCTGCGTCCCTCCTTCACTCAATACTCTAGTACAGGA |
| | 214494 | CGCCGTCGATGTGAACTCTTGGGCGAGATCAGCCTGTTATCCCCAGGGTA |
| | 214495 | AGTCGTTTCCAACTGTTGTCCCCCACTCCAGGGCAGGTTACTCACGCGTT |
| | 214496 | GCATGCTTAAAGTTCGGCGGCTACGGAATTTCAACCGTATGTGCATCGAC |
| | 214497 | ATTACCGCGGCTGCTGGCACGGAATTAGCCGGTCCTTATTCTTATGGTAC |
| | 214498 | CGCACAGCCCTGTGTTTTTGTTAAACAGTTGCCTGGACCTATTCTCTGCG |
| | 214499 | CATAATTTTATTTTCTTCTCCTACGGGTACTGAGATGTTTCACTTCCCCG |
| | 214500 | ACCTTGGGCGGACGAACCTTCCCCAAGAAACCTTAGATTTTCGGCCATTA |
| | 214501 | TACTATCAGGTTCGGCTCTCAAGGTGGATTTGCCTGCCTCGATCTGCGCC |
| | 214502 | CTGTACATGCAATACCAAGCTCCAGTACCAAACTGGAGTAAAGCTCCATG |
| | 214503 | TGCTTGACCACGGAAAACCACCTCCGCGGCCGGCTCCCATTCCGTGTCAC |
| | 214504 | CAGTAACCCGCAAGGCTGCACCTAAATGCATTTCGGGGAGTACGAGCTAT |
| | 214505 | AAGCCAACATCCTGGTTGTCTACGCAATTGCACATCCTTTTCCACTTAAC |
| | 214506 | CACATCTTACGACGGCAGTCTCGACAGAGTCCCCAGCATCACCTGATGGT |
| | 214507 | TTATAGTTACGGCCGCCGTTCACTGGGGCTTCGATTCAATGCTTGCACAT |
| | 214508 | CATCTTTACTCGTACTGCAATTTCGCCGAGCTCCTGGTCGAGACAGTGGG |
| | 214509 | ACACCGAGCCATGCAGCTCTGTGCGCTTATGCGGTATTAGCAGTCATTTC |
| | 214510 | AGGTCCCGCGCTCCCCACCACCGTCCCCGTCAAAGACGGGGTTCGGGATG |
| | 214511 | ATCGAGCTCACAGCATGTGCATTTTTGTGTACGGGGCTGTCACCCTGTAT |
| | 214512 | GGAATTTCTCCCCTAGCCACAAGTCATCCGCTAACTTTTCAACGGTAGTC |
| | 214513 | GCTCTACCTCCAAGACTCTTACCTTGAGGCTAGCCCTAAAGCTATTTCGG |
| | 214514 | TTATAGTTACGGCCGCCGTTTACTGGGGCTTCAATTCAATGCTTCTCTTG |
| | 214515 | CTTCAACCTGGACATGGATAGGTCACCCGGTTTCGGGTCTGCACACACTG |
| | 214516 | GAGGCTAGCCCTAAAGCTATTTCGAGGAGAACCAGCTATCTCCGGGTTCG |
| | 214517 | TGGGCTGTTTCCCTTTCGACTACGGATCTTAGCACTCGCAGTCTGACTGC |
| | 214518 | CTCCGGCCTATCCTACACATCGATTGCCCAAATTCAATGTAAAGCTATAG |
| | 214519 | CCACTTCACCTAACAACAATGCAAAAAGGGCGTGCCACTGGTAGATGACA |
| | 214520 | ACCCTCAGGTCATCCAGAAGCTTTTCAACGCTTATTGGTTCGGTCCTCCA |
| | 214521 | AGTATCCCTTCCTGCAGGTACTGAGATGTTTCACTTCCCTGCGTACCCCC |
| | 214522 | ACTTGGTATCCCTTCGGCTCCGCACCTTAAGTGCTTAACCTCGCCAGTAT |
| | 214523 | TCGGATACGTGTGTCGTCACACTTAACCTTGCCGGCAAAGGCAACTCGTA |
| | 214524 | GGATCACTAACTCCAACTTTCGTTACTGCTCGAACTGTCGCTCTCGCAGT |
| | 214525 | CGAACGCCTTAGTATTTTCAACCTGACTACCTGTGTCGGTTTGGGGTACG |
| | 214526 | TTCTGCTTCTGCCCGTACACGTTGCTCCCCTACCCAGAAGTTTCCTTCTG |
| | 214527 | TCACGGTACTAGTTCGCTATCGGTCAGACAGGTATATCTAGGCTTACCCC |
| | 214528 | ACTTCTTACAAAGCTCCGACCGCTTGTAGGCGCATGGTTTCAGGGACTAT |
| | 214529 | TCTTTAAAGGATGGCTGCTTCTGAGCCAACCTCCTAGTTGTCTGGGCATC |
| | 214530 | CCCCATTGGGGCCCACAACACCGCACACACAACCCCTACCAAGTATCACA |
| | 214531 | CTCAACTTCAACCTGCTCATGGCTAGATCACCCGGTTTCGGGTCTGCAAC |
| | 214532 | GCATACGCCACACGGCTTATGCTCGCCACCCGCCACTGACTCGCAGACTC |
| | 214533 | GTTCGTCTATATGCCCGCACCTCACTGCGCCATGCCGGCAGACATGACCA |
| | 214534 | ATCTGGGCTGTTTCCCTTTTGACAATGACATTTATCTGACACTGTCTGAC |
| | 214535 | CTATTAGTAGCAGTCAGCTCCATGTGTTACCACACTTCCACCCCTGCCCT |
| | 214536 | TTTCACAACTGACTTAAACATCCATCTACGCTCCCTTTAAACCCAATAAA |
| | 214537 | CCGTTGAATTTTCGGCGCAGAGTCACTCGACCAGTGAGCTATTACGCACT |
| | 214538 | TCCTTAACGAGAGTTCGCTCGCTCACCTGAGGCTACTCGCCTCGACTACC |
| | 214539 | CCACTCCGTCGATGTGAACTCTTGGGAGTGATAAGCCTGTTATCCCCAGG |
| | 214540 | CAACAGGATGAAGTTTAGCGGATTTTCTCGGGAGTATGATTACATGCGCT |
| | 214541 | GACGGGCTGCGTGCTTGACCACGGAAAACCACCTCCGCGGCCGGCTACCC |
| | 214542 | CGGATTTGCCTATGATGCGCGCTGCGTGCTTGACCACGGAAAACCACCTC |
| | 214543 | CTGAGTTTGATAAGCTTCGCTAACCTCTCGGCCGCTAGGCTATTCAGTGC |
| | 214544 | TGCAGCACCTGTCTCACGGTTCCCGAAGGCACATTCTCATCTCTGAAAAC |
| | 214545 | AGGCTAGCCCTAAAGCTATTTCGGGGAGAACCAGCTATCTCCGAGTTCGA |
| | 214546 | GACGTCCTATCTCTAGGATTGTCAGAGGATGTCAAGACCTGGTAAGGTTC |
| | 214547 | GTTTTGACTACAGGGCTGTTACCTCCTATGGCGGGCCTTTCCAGACCTCT |
| | 214548 | CTGGGGCTTCAATTCAGATCTTCGCTAACGCTAAACCCTCCTCTTAACCT |
| | 214549 | CCTTAGTATATTCAACCCGACTACGTGTGTCCGTTTACGGTACGGGTACC |
| | 214550 | CTATACATCATCTTACGATTTAGCAGAGAGCTGTGTTTTTGATAAACAGT |
| | 214551 | CTAACAATGTCCCCCGACTCGATTCAGAGCCGCAGGTTAGAATTCCAATA |
| | 214552 | TTTGGCCTCTTCCGCGTTCGCTCGCCACTACTTACGGAATCTCAGTTGAT |
| | 214553 | CCCGCCAACTGGCTAATCAGACGCGGGTCCATCTTATACCACCGGAGTTT |
| | 214554 | GCTACTTGGGACACGCGATCGGAAGACGGCAAGCGTCCAGGTACGGGGCT |
| | 214555 | CATCACCGGGGATGAGCTACCTCACTGCGTCCCTCCGCAGCTTGCCTACT |
| | 214556 | ACAACTTAATACCCGATTATTATCCACGCCAGACTCCTCGACTAGTGAGC |
| | 214557 | CTCTCAGACCAGTTACGGATCGTCGCCTTGGTAGGCCTTTACCCCACCAA |
| | 214558 | TCACGTAGTCTGACTGCTGATCATCAATTAGCCGGCATTCAGAGTTTGAT |
| | 214559 | TAGGTCACCCGGTTTCGGGTGTACTGCATGCAACTTTACGCCCTTTTCAG |
| | 214560 | TACTTTAGTTCGCTCCACATCACGGCTTCGTCTCATGCACAGCGGATTTG |
| | 214561 | CTTACGGGGCTTTCACCCTCTCTGGCAGGCTTTCCCAAAAACCTTTCTGC |
| | 214562 | GGCCGGGCTTTCGATCCCGTTCTTCTATCCTCTCTCTTGCCATATCATGG |
| | 214563 | ACGGCTTCTACTCGTATACAACGCTCCCCTACCACTATAGTTTCCTACAA |
| | 214564 | ATCGAGTTTTCTTTCTCTTCCTCCGGCTACTTAGATGTTTCAGTTCACCG |
| | 214565 | GCTTTACATACCGAAATACTTCTTCACTCACGCGGCGTCGCTGCATCAGG |
| | 214566 | TCCCTTCTGCCTTTGCACTCTTCTAATGGTTTCCGACCATTATGAGGGAA |
| | 214567 | CTCCATCAGGCAGTTTCCCAGACATTACTCACCCGTCCGCCACTCGTCAG |
| | 214568 | TGCCAAACCTCCCCGTCGATGTGAACTCTTGGGGGAGATAAGCCTGTTAT |
| | 214569 | GCCTGGACCTATTCTCTGCGCCTCACATTACTGTGAGGACCCTTTATCCC |
| | 214570 | ACCTTTACACCTGCATCCTATCAACGTCGTAGTCTACAACGACCCTCAGA |
| | 214571 | GTATTCATTAACGCTAGAAGCTTTTCTTGGCAGAGTGACATCACTAGCTT |
| | 214572 | GCTGTTGGTCCGGATTGTTCTCCTTTAGGACATGGACCTTAGCACCCATG |
| | 214573 | AAAAACCCTCCCCCCCCCCCCTTCCCCTCCGCGGCCACCTTTCCCCCCCC |
| | 214574 | CTGTCGGTACCCGATACGGGCCCTCAAGCATCCAGTAGCTCTACCCCCCG |
| | 214575 | ATCTACGCATTTCACCGCTACACTAGGAATTCCGCTTACCTCTGTTGCAC |
| | 214576 | TCTGTCCCACCTTCGGCGGCTGGCTCCTAAAAGGTTACCTCACCGACTTC |
| | 214577 | TGACCAAGGGTAGATCACTTGGTTTCGCGTCTACTCCTTCCGACTAATCG |
| | 214578 | TGTGCACTTGCACTCGCCACCCGATTGCCAACCGGGCTGAGCGGACCTTT |
| | 214579 | CAGCCTCACTCCCAGGCTGTAAAATATGCCCCTTCGGAGTTTGATAAGGT |
| | 214580 | ACGCTTCCACTAACACACACACTGATTCAGGCTCTGGGCTGCTCCCCGTT |
| | 214581 | CTGTCAAGGTCGACTCTCCCTGCGGATTTGCCTACAGGAATCTACATCTA |
| | 214582 | CCTGTGTTTTTGGTAAACAGTCGCTACCCCCTGGCCTGTGCCACCCCCCG |
| | 214583 | ATCTGATAGCGTGAGGTCCGAAGATCCCCCACTTTCTCCCTCAGGACGTA |
| | 214584 | ACACTTTGGGACCTTAGCCGGTGGTCTGGGCTCTTTCCCTTTTGACTACC |
| | 214585 | CTACAAGGGATCTTACCTGATTGAATCAGTGGGATATCTTATCTTTGGGT |
| | 214586 | CTGAAGGGTAACCCCACATAACCAGGGCCAGGTTTCCCCATTCGGACATC |
| | 214587 | TCAGTCCGCGGCGCTGTCACGCCTCCGTCTCCACGTCACTCCTTAAGGTA |
| | 214588 | TTAACAAGGGTTCTCCCGTTCGTCTCAGGATTCTCTCCTCGCCCACCTGC |
| | 214589 | CTAACATCCTAGTTGTCTGTGCAACCCCACATCCTTTTCCACTTAACAAT |
| | 214590 | GATAAATCTTTCCCCCGTAGGGCACATTCGGTATTACTCCCAGTTTCCCG |
| | 214591 | GTTTACAATCCGAAGACCTTCTTCCCACACGCGGCGTTGCTGCATCAGGG |
| | 214592 | CGGCGCACTGCAGCTACCTGTCTGCGTCACCCCTGTTAACACGCTTGCCT |
| | 214593 | ATGAAGCTGGAATCGCTAGTAATCGTATATCAGCAATGATACGGTGAATA |
| | 214594 | CGGATTTGCCTATGGGACGGGCTGCGTGCTTGACCACGGAAAACCACCTC |
| | 214595 | GGATGACCCCCTTGCCGAAACAGTGCTCTACCCCCGGAGATGAATTCACG |
| | 214596 | GGTACGGGTAACATATACTATAACTTAGAAGATTTTCTCGGAAGTCGACT |
| | 214597 | CTTTGTAACTCCGTACAGAGTGTCCTACAACCCCAAGAGGCAAGCCTCTT |
| | 214598 | TCTTACTTCTTGCGAATGGGAGATCTCATCTTGGAGTAGGCTTCGTGCTT |
| | 214599 | GTCAAGCTCCCTTATACCTTTACACTCTGCGATTGATTTCCAACCAATCT |
| | 214600 | CCACCTATCCTACACATCAAGGCTCAATGTTCAGTGTCAAGCTATAGTAA |
| | 214601 | AAAAGCAGTTTACAACCCATAGGGCCGTCATCCTGCACGCTACTTGGCTG |
| | 214602 | TGAGGGCACCTTTAGAAGCCTCCGTTACACTTTTGGAGGCGACCACCCCA |
| | 214603 | ACGCTCTAACCTTATGGTAACCGGATTTGCCTGGTAACCAGCCGCTTCGC |
| | 214604 | GCTTCCAAGCCAACATCCTAGCTGTCTTAGCAATCTGACTTCGTTAGTTC |
| | 214605 | TGGCCGTTCACCCTCTCAGGCCGGCTATGGATCGTCGCCTTGGTAGGCCG |
| | 214606 | TGAGCCAACATCCTGGTTGTCTTCGAAATCCCACATCCTTTTCCACTTAA |
| | 214607 | CTAGAGAGTATTTAGGGTTAGGAGATGGTCCTCCCAGATTCCGACGAGAT |
| | 214608 | GCCTTTCGGCCTCGCGTTAGGTCCCGACTTACCCAGGGCGGACGAACCTT |
| | 214609 | GTCAAACTGCCCACCTGACACTGTCTCCCCGCCCGATAAGGGCGGCGGGT |
| | 214610 | TGGAGTAAAGCTCCATGGGGTCTTTCCGTCCTGGCGCAGGTAACCAGCAT |
| | 214611 | TTTCTTCTCCTACGGGTACTGAGATGTTTCACTTCCCCGCGTAACCCCCA |
| | 214612 | ACCAGCTATGGATCGTCGGCTTGGTAGGCCATTACCCCACCAACTACCTA |
| | 214613 | GGGGCAAGTTTCGTGCTTAGATGCTTTCAGCACTTATCTCTTCCGCATTT |
| | 214614 | CACCAGTGTCGGTTTGGGGTACGGGCGGCCATAGCCCTCACGCCGAGGCT |
| | 214615 | GACGTTCTGAACCCAGCTCGCGTGCCGCTTTAATGGGCGAACAGCCCAAC |
| | 214616 | GGTTAGAATTCCAATATCGCAAGGATGGTATCCCAACGGCCTCTCCGCCA |
| | 214617 | AGGTTACCCACGCGTTACTCACCCGTCCGCCACTAGAAACAATCTAAATC |
| | 214618 | CAGGTGTCACCCCATATACGTCATCTTTCGATTTAGCATAGAGCTGTGTT |
| | 214619 | TCTTTCGGCGAGGGGGTTTCCCACCCCCTTTATCGTTACTTATACCTACA |
| | 214620 | CTTAGGACCGTTATAGTTACGGCCGCCGTTTACCGGGGCTTCGATCAAGA |
| | 214621 | CCACTTAGTGATGATTTGGGGACCTTAGCTGGCGGTCTGGGTTGTTTCCC |
| | 214622 | TCCCCCATTCGGACACCTCCGCTTCTTCGCTTCCTTACAGCTTCACGGAG |
| | 214623 | ATAGATCACCCGGTTTCGGGTCTGCCCCCACTGACTCTGGCCCTCTTAAG |
| | 214624 | GCCTATCAAACACGTGTTCCACATGCGGGCTTCAGGACCCCGAAGGGCCC |
| | 214625 | CCATTTCTGACTGTTATCCCCCTGTATAAGGCAGGTTGCCCACGCGTTAC |
| | 214626 | CATCATCTGTATGGCATTCGGAGTTTGATATCCCTTAGTAAGCTTTGACG |
| | 214627 | GTTTGGGGTACGGGCGGCTAAAACCTCGCGCCGATGCTTTTCTAGGCAGC |
| | 214628 | GCGATGGCCCTTCCATACGGTACCACCGGATCACTAAGCCCGACTTTCGT |
| | 214629 | GAGTTAACCCCGGCGGTCCCCCGTGAGTTCCCACCATAACGTGCTGGCAA |
| | 214630 | GGATAATCGGCGGACGGGATTCCCACCCGTCACACGCTACTCATGCCTGC |
| | 214631 | TACCTCTTCGTTATGATATGTCCGCAACCCCAATAAAGAAAACTTTATTG |
| | 214632 | ACGTGTCCGGCGGTACTCTGGATTCAGCTGGCGGATCTTCTCTTTCGCAT |
| | 214633 | TCGAGACCAGACTTCGTTAGACTAACTCAGACAGGATTCCGGGACCTTAG |
| | 214634 | TGGCCGTTCAACCTCTCAGTCCGGCTACCAATCGTCGCCTTGGTGGGCCG |
| | 214635 | TATAAGTCAAGGCTGCACCTAAATGCATTTCGGGGAGTACGAGCTATCTC |
| | 214636 | CTACTGTTTCACCGCGTATACAACGCTCCCCTACCCAGCATGTAAACATG |
| | 214637 | TTATAGTTACGGCCGCCGTTTACTGGGGCTTCAATTCACACCTTCGACAA |
| | 214638 | GGATGGACCCCTCACCCAAACAGTGCTCTACCTCCATGATTCTTAATGTC |
| | 214639 | TTGGGACCTTAGCTGCGGGTCTGGGCTCTTTCCCTTTTGACTATCCAACT |
| | 214640 | GGCTCTGACTACTTGTAGGCACACGGTTTCAGGATCTCTTTCACTCCCCT |
| | 214641 | TCGCTACTCATTCCGGCATTCTCACTCGTGTACAGTCCACCGCTGCTTTC |
| | 214642 | CCTCCCCCCCCCCCCCCCCCCCCCCCCCTTCCCCCCTCTCCTCCCCCTTC |
| | 214643 | TAACACCCCATAACAGGTGCCAGGTTTCCCCATTCGGACATCCTCGGATC |
| | 214644 | ACCTCGACACGGACGGTGACAAGCCGGTACCAGAATATCAACTGGTTACC |
| | 214645 | ATAGATCACCCGGTTTCGGGTCTACTCCGGCTGACTCGCTCGCCCTATTC |
| | 214646 | TAAATGATGGCTGCTTCTAAGCCAACATCCTGGCTGTCTGGGCCTTCCCA |
| | 214647 | CAGCTTATAGGGTTGCGTACTTCACTACAACCCAACCTTGATGCTTGCAC |
| | 214648 | GCTTGGGCCTTTTCACTGCGGCTGACTTATCGCCAGCGCCCCTTCTCCCG |
| | 214649 | TGAGGTCGGCTTCACGCTTAGATGCTTTCAGCGTTTATCCGTTCCGCACT |
| | 214650 | CTCCGGGTACTGTCAGGTTCGACTCTCAGGGCGGATTTGCCTACCCCGAT |
| | 214651 | GCTTGGGCCTCTTCACTGCGGCTTAATTGCTTAAGCACTCCTTCTCGCTA |
| | 214652 | TTTATCCCGAAGTTACAGGGTCAGTTTGCCTAGTTCCTTAACCGTGAATC |
| | 214653 | GTAGTTAGCCGGAGCTTCCTCCTAAAGTACCGTCATTATCGTCCTTTAAG |
| | 214654 | TCTTTCGGCGAGGGGGTTTCCCGCCCCCTTTATCGTTACTTATACCTACA |
| | 214655 | GGATGTACTAGCAGCTTTTCTCGCCAGCGTGAACTCACTCGCTTCCCTAC |
| | 214656 | TTAGTATCAGTGCTTTATCAGGGGCGCATATACTCGGGTACCAGAATATC |
| | 214657 | GCTTGGCGGCGTCCTACTCTCACAGGGGGAAACCCCCGACTACCATCGGC |
| | 214658 | AGATTCACGCAGAATTCCTCGTGCTCCGCGCTACTCAGGATACTACTATG |
| | 214659 | TATCAACCTGATCATCTTTCAGGGATCTTACTTCCTTGCGGAATGGGAAA |
| | 214660 | TCAATAGGCACGCCACCACACTCTTATGGAGCGGTGACTGCTTGTAAGTC |
| | 214661 | CTACTATATTTCGGTCCCTTACGCCCGGGGCAACCATCGCCCGGGATAAC |
| | 214662 | TGCCATGACTGCTTGTAAGTCCACGGTTTCAGGTTCTCTTTCACTCCCCT |
| | 214663 | TCCATTTGCGCAGCACCAGTAATCATGTTCTTAACATAGTCAGCATGTCC |
| | 214664 | TCTCAGTCCCAATGTGGCCGGTCACCCTCTCAGGTCGGCTACTGATCGTC |
| | 214665 | TGGCCGTTCAACCTCTCAGTCCGGCTACTGATCGTCGCCTTGGTGGGCCT |
| | 214666 | TTATAGTTACGGCCGCCGTTTACCGGGGCTTCAATTCGGAGCTCTCACTC |
| | 214667 | TAGTGAAAGGTAGATTTTCTGACCCTTTCGACCTGAACGTACCAACCAGC |
| | 214668 | TCTTGGCAGTGTGACATCACTAACTTCGCTACTAAACTTCGCTCCCCATC |
| | 214669 | ACCTGCTTTCGCACCTGCTCGCGCCGTCACGCTCGCAGTCAAGCTGGCTT |
| | 214670 | TCGGAGTTTGATATTCTTCGGTAAGCTTTGACGCCCCCTAGGAAATTCAG |
| | 214671 | ACCCACCGAGTGGGCGCCCATCAGGTCTCAAGCACATAGCCGGCGGATTT |
| | 214672 | TACGGGTGCCGCATGGATAAGTTTAGCGGATTTTCTCGGGAGCATGGTTA |
| | 214673 | TTCAAACAACCATCCGGTATTAGCCCCGGTTTCCCGGAGTTATCCCAGTC |
| | 214674 | TCCTTAACCACGCTGCATACCATAACTCGCCGGACCATTCTACAAAAGGT |
| | 214675 | CCGGCACCGGGCAGGTGTCAGGCTGTATACGTCATCTTTCGAGTTTGCAC |
| | 214676 | CAGGAATATTCAGGCTTACCCAACGGTCTGGGCGGATTCGCACGGGGTTC |
| | 214677 | TTTATCCCGAAGTTACAGGGTCAGTTTGCCTAGTTCCTTAACCGTGAATC |
| | 214678 | CTTCTGCAATTGCACTCGTCGATTGGTTTCCATCCAATCTGAGCGTACCT |
| | 214679 | TCGGTTTGCCCTCTTCCGCGTTCGCTCGCCACTACTTACGGAATCTCGTT |
| | 214680 | AAGCTCCATGGGGTCTTTCCGTCTTGTCGCGGGTAACCGGCATCTTCACC |
| | 214681 | CATCGGCCTCACCGTTCGGCTGAGCCTTAGGACCCGACTAACCCTGATCC |
| | 214682 | CCTCGCCATACACGCCGCACGGATTTGCCTATGCGACTGGCTGCGTGCTT |
| | 214683 | CCTGTCGCGGGTAACCTGCATCTTCACAGGTACTATAATTTCACCGAGTC |
| | 214684 | TCAGCCTTATGGGAAACGGATTTGCCTATTTCCCAGCCTAACTGCTTGGA |
| | 214685 | TTTCACAACACGCTTAAAAGGCGGCCTACGCTCCCTTTAAACCCAATAAA |
| | 214686 | CCCCGCGGTACTCTGGATCCTGCTAGCTCTCGCTCCTTTTCGTCTACGTG |
| | 214687 | ATCGGTTCACACACTCACCCACCCCAGAAGCATCAAAAACACTCCCAAGA |
| | 214688 | TAGAAAGGAGGTGATCCAGCCGCACCTTCCGATACGGCTACCTTGTTACG |
| | 214689 | GCCCATTGTCCAATATTCCCCACTGCTGCCTCCCGTAGGAGTCTGGACCG |
| | 214690 | TCACCTTTCCCTCACGGTACTGGTTCGCTATCGGTCTCTCGGGAGTATTT |
| | 214691 | CGAAGTTACGGGGTCATTTTGCCGAGTTCCTTGACAATGCTTCTCCCGCC |
| | 214692 | AGATCCTCTCAAATTTCCTACGCCCGCGACGGATAGGGACCGAACTGTCT |
| | 214693 | TCTCAGTCCCAATGTGGCCGGTCACCCTCTCAGGTCGGCTACTGATCGTC |
| | 214694 | GGCAACCCAACAACCCACACATCATCATCTTCAGCTACAGGACTCTCACC |
| | 214695 | GCACTATTGCCTTGTCCCGGAGGACGCGGCATACTGTCAGGTTCGAATCA |
| | 214696 | CCGTGGCTTTCTGGTTAGGTACCGTCAAGGTACCGCCCTATTCGAACGGT |
| | 214697 | ATACTATCAGGTTCGACTCTTATCCCGGATTTGCCTGGGATAATCAACAT |
| | 214698 | TAAGTCCTTAACCTTGCTGCATACAATCGCTCGCCGGACCGTTCTACAAA |
| | 214699 | ATCTGGGCTGTTTCCCTTTTGACAATGACATTTATCTGACACTGTCTGAC |
| | 214700 | AGAGTAACCATAACACAAGGGTAGTATCCCAACAACGCCTCCTCCGAAAC |
| | 214701 | TGGACAGGATTCTCACCTGTCTTACGCTACTCATACCGGCATTCTCACTT |
| | 214702 | GCCCGGCTACCTTCCTGCGTCACACCTGTTAATACGCTTGGCTCCCCAGT |
| | 214703 | GTCAAGCTCCCTTATACCTTTACACTCTGCGAATGATTTCCAACCATTCT |
| | 214704 | CCCAACCCTTGGAACATACTACAGCCCCAGGTGGCGAAGAGCCGACATCG |
| | 214705 | TCTTTCGGCGAGGGGGTTTCCCACCCCCTTTATCGTTACTTATACCTACA |
| | 214706 | GGGTGTTCCCCTTTTGCCCGCGGAACTTATCTCTCGCGGACTGACTCCCA |
| | 214707 | ACCCGGTTTCGGGTCTATGGCATACAACTTCTCGCCCTTGTCAGACTCGC |
| | 214708 | CTGCCTGGCTTACGCCTACGGGGCTTTCACCCTCTCCGGCGCCGGCATTC |
| | 214709 | GCTGCGGGGCTGAGCCCCTTAACCTCGCCGGAAAAAGTAACTCGTAGGTT |
| | 214710 | AAGGATGGCTCTCTTCAAATCTCCTGCGCCCGCGACGGATAGGGACCGAA |
| | 214711 | CAGGCCCCACAACACCGCACACACAACCCCCGCCGGGTATCACATGCACA |
| | 214712 | CCCCTACGGATCCATGCCTTGGTGGGCCATTACCCCACCAACTAGCTAAT |
| | 214713 | ACTTAGCACTCATCGTTTACGGCGTGGACTACCAGGGTATCTAATCCTGT |
| | 214714 | TATCCATCGAAGACTAGGTGGGCCGTTACCCCGCCTACTATCTAATGGAA |
| | 214715 | CAGGCGTCAGCTCGTATACGTCATCTTTCGATTTAGCACAAACCTGTGTT |
| | 214716 | TGGCCGTTCAACCTCTCAGTCCGGCTACCGATCGCGGTCTTGGTGAGCCG |
| | 214717 | CCTGTGTTTTTGCTAAACAGTCGCCTGGGCCTATTCACTGCGGCTCTCTC |
| | 214718 | ACGCCTTTCGGCCTGACCTTAGCTCCCGACTTACTTGGAGCGGACGAACC |
| | 214719 | GGTCTGGGCTCTTTCCCTTTTGACTGCCCAACTTATCTCGTGCAGTCTGA |
| | 214720 | GAATGAATGGCTGCTTCTGAGCCAACATCCTAGTTGTCTTAGAGATCCCA |
| | 214721 | CCCCATCATGCCTCAACCTTCACGCCCAGCGGATTTACCTACCAGACAGT |
| | 214722 | AAAAGTACGCGGTTCATCATATAAAGATGTTCCACAGCTTGTAAACACAG |
| | 214723 | ATCTGAAGTCTTCTCGTTTAACATACAGGACTATTACCTTCTGTGGTGAG |
| | 214724 | GGTCACACCCTTTTGAAGTGTCCCTTTGCTTAAATTACAGATGGTTACGG |
| | 214725 | CAGCTTATCACGTCTTTCATCGGCTCTTAGTGCCAAGGCATCCACCCTGC |
| | 214726 | TTCCATTCGGCACCGCCGGATCACTATTCCCGACTTTCGTCCCTGTTCGA |
| | 214727 | TCCAGGTTCGATTGGCATTTCACCCCTACCCACACCTCATCCCCGCACTT |
| | 214728 | TACACCTTCTGCGTACATAGAACGCTCTCCTACCATCCCCTAAGGGATCC |
| | 214729 | GCTTGCGCTAACCTCTCCTCTTAACCTTCCAGCACCGGGCAGGCGTCAGC |
| | 214730 | CGCCCGTTAGTACCGGTCGGCTCCACCCCTCGCGGGGCTTCCACCTCCGG |
| | 214731 | CTCCGGGACCTTAGACGGCGGTCTGGATTCTTCTCCTCTCGGGGACGGAC |
| | 214732 | TGGTTAAGTCCTCGATCGATTAGTATCTGTCAGCTCCATGTGTCGCCACA |
| | 214733 | TAAGTCCTTAACCTTGCTGCATACAATCGCTCGCCGGACCGTTCTACAAA |
| | 214734 | ACCGGACTTTCCATTTCCGGCCCATGTTTCCCTCCCGTGTCCCCACAGTT |
| | 214735 | CGGCTCCCACCTATGCTACGCAGAAGAATCCGGATATCAATGCCAGACTA |
| | 214736 | ACCCCACATCCTTTTCCACTTAACATATATTTGGGGACCTTAGCTGGTGG |
| | 214737 | CCACACCACTTCACCTAACAACAACACACAAGCACGATGATGGTAGTCAC |
| | 214738 | TCATCCCCGCACTTTTCACGTACGTGTGGTTCGGACCTCCACGACGTCTT |
| | 214739 | CCCTTCAAAGCCTCCGACCTATCCTACACATCACGTGCCCAGATTCAATG |
| | 214740 | CTTCACCTAACAACAATGCGCAAGCAGGACGTCAGTAGCCATCCTCATCA |
| | 214741 | GGGGTACGGGCGGCAACGCGCCTGACGCCGAAGCTTTTCTCGGCACCACG |
| | 214742 | ATGGCTAGATCACCGGGTTTCGGGTCTATACCCTGCAACTTAACGCCCAG |
| | 214743 | ATTAAACCACATGCTCCACCGCTTGTGCGGGCCCCCGTCAATTCCTTTGA |
| | 214744 | GCCGGCTTTCCCAAAGCCGTTCTGCTACCTCTCGCGGATCAATTATGCGG |
| | 214745 | ACGCCTTCCGGCCTCACCTTAGCTCCCGACTAACTTGGAGCGGACGAACC |
| | 214746 | ACACCACGCGGCGATACCAACCCGAAGGAAGGAACCACCACGAGGCGGAG |
| | 214747 | CCGAACCCCGAGATGCACGCATCTCGGTTTGGCCTCTTTCGCGTTCGCTC |
| | 214748 | GGGACTTCATCCTGGCCAAGTGTAGATCACTTGGTTTCGCGTCTACCCCC |
| | 214749 | AGCCCTCGACCTATTAGTACTGCCAAGCTGAATGCCTCACGGCACTTACA |
| | 214750 | GGGAGCGGGATTACCTTCACTATCAATCCACCCGAAGGTTTCATGTACTA |
| | 214751 | CACGCGGGATTCCACGAGGCCCGCGCTACTTGGGACAACACGATCGGAAG |
| | 214752 | CCTACACCCTTCAACCATCTATTCCGTCAGATGGCGGCACTGTCACTACT |
| | 214753 | CCCCGTACCTGTTCTCGATACCAGGTTAGAACCCCGGTCACACAAGAGTG |
| | 214754 | GTTTCACGTGTCTGGCCGTACTCTGGATCCTGCGCAGCTCTCTCCGTTTT |
| | 214755 | TTCCCGCTTAGATGCTTTCAGCGGTTATCCCTCCCGAACGTAGCCAACCG |
| | 214756 | GCACTCCCACAGCTTGTAGACACAGGGTTTCAGGTTCTCTTTCACTCCCC |
| | 214757 | CCTGGCCAAGGGTAGATCACTTGGTTTCGCGTCTGCCACTGCCGACTATA |
| | 214758 | CCGCGAGGGACCTCACCTACATATCAGCGTGCCTTCTCCCGAAGTTACGG |
| | 214759 | AAGCTCCATGGGGTCTTTCCGTCTTGCCGCAGGTAACCGGCATCTTCACC |
| | 214760 | CGTCGGCTTGGTGGGCCGTTACCTCACCAACTACCTAATCCAACGCGGGT |
| | 214761 | GCTCCCACCTATCCTGTACATGCAATACCAAGCTCCAGTACCAAACTGGA |
| | 214762 | ACCGGACTTTCCATTTCCGGCCCATGTTTCCCTCCCGTGTCCCCACAGTT |
| | 214763 | CAGTTCCCCGGGTCTGCCTTCTCATATCCTATGAATTCAGATATGGATAC |
| | 214764 | GGTCCCGGCAGATTCGCGCAGGATTCCTCGTGTCCCGCGTTACTCAGGAT |
| | 214765 | GTATTAACTTTACTCCCTTCCTCCCCGCTGAAAGTACTTTACAACCCGAA |
| | 214766 | GGGGGCGGGGAGCGGGGCGTGGGCGGGAGGAGGGGAGGAGGCGTGGGGGG |
| | 214767 | CACGAGGCCCGCGCTACTTGGGACACGCGATCGGGAGACGGCAAGCGTCC |
| | 214768 | CGTTTATCCCCTCCCTACTTAGCTACCCAGCGATGCTCTTGGCAGAACAA |
| | 214769 | CCTCTTAACCTTCCGGCACCGGGCAGGCGTCAGAGCGTATACAGCGGCTT |
| | 214770 | ACCTTGGGCGGACGAACCTTCCCCAAGAAACCTTAGATTTTCGGCCATTA |
| | 214771 | TTCGTTCGCCACTACTAGCAGAATCATAATTTTATTTTCTTCTCCTACGG |
| | 214772 | GTTTCTCGCATGCCTCTCGCTACTCATACCGGCATTCTCTCTTGTGCAGT |
| | 214773 | CCTATCAACGTCGTCGTCTTCAACGTTCCTTCAGGACCCTTAAAGGGTCA |
| | 214774 | CTGTTATCCCCAGGGTAGCTTTTATCCGTTGAGCGACGGCATTTCCATTC |
| | 214775 | CAACAATATATGGAACACCTACCTGGCGAGACAATAGAATGTGTTCCCTC |
| | 214776 | TTATAGTTACGGCCGCCGTTTACTGGGGCTTCAATTCAATGCTTCTCTTG |
| | 214777 | ACAACAGAGCTTTACGATCCGAAAACCTTCATCACTCACGCGGCGTTGCT |
| | 214778 | CCCGTTCCACGGGTTAGAATCCAAACAAATAAAGGGTCGTATTTCAACAG |
| | 214779 | CCCCCTTCCCCCCTCTCCTCCCCCTTCCCCCTTTCGCGCCCCCTTTTCCC |
| | 214780 | TGGTGTTCCAACCAATTCGGCTTGGGGGGATGGATCTTAAAAACTGGTCC |
| | 214781 | CTCGTGTCCCGCCGTACTCAGGATCCTGCTTGGCATCAAGTGAATTTCAA |
| | 214782 | AGCTTCTACACCCTTCAACCATCTATTCCGTCAGATGGCGGCACTGTCAC |
| | 214783 | CCGATTAGTACCAGTCAACTCCGTACATCACTGCACTTCCATCCCTGGCC |
| | 214784 | CGCTTGAACCACACATCAGGCCCCACGGCTTGCCACCATGTTAACCCGAA |
| | 214785 | TGGCGAGACAATAGAATGTGTTCCCTCGTTTGTGGCATAGGACCATCAGC |
| | 214786 | CGTCCATCCCGGTCCTCTCGTACTAGGGACAGCTCCTCTCAAATATCCTG |
| | 214787 | TCGAGGTGCCAAACCTCCCCGTCGATGTGAACTCTTGGGGGAGATAAGCC |
| | 214788 | CTTAACAACTTAACCTCGCTGCACACAGTAACTCGCCGGCCCGTTCTACA |
| | 214789 | GTCAACAGGTAGTATTCAGGCTTACCAGGTGGTCCTGGCAGATTCACACG |
| | 214790 | AGGCACGCCGTCACACATTGCTGTGCTCCGACCGCTTGTAGGCGTATGGT |
| | 214791 | TCCCTTTCCCCCTTCCCCCCCCCCCCCCCCCCCCCCCCCTTTCCCCCCCC |
| | 214792 | AACCATGACTTTGGGACCTTAGCTGGCGGTCTGGGTTGTTTCCCTCTTCA |
| | 214793 | TGCCATTACACTCTATGAGACCGGTTACCAATCGGTCCGAAGGGCACCTT |
| | 214794 | GATTGGAATTTCTCCGCTACCCACACCTCATCCGCTACCATTTCAACGGG |
| | 214795 | TTCTCGTGTCCCGCGGTACTCTGGATCCTGCTCAGTCTGCTCTGTTTTCG |
| | 214796 | GTAAACCCCCACAACAGCTATGAATTCACTGAAGGGTAACACCCCATAAC |
| | 214797 | TCCCGAAGTTACAGGGTCAATTTGCCTAGTTCCTTAACCGTGAATCACTC |
| | 214798 | CCCCCGACGGGTATCACACGCGCAAGGTTTGGCCATCATCCGCTTTCGCT |
| | 214799 | CCCTTGTCTCAGTGCCCATCTCCGGGCTCCTCCTTCCAGAGCCCGTACCC |
| | 214800 | TCAGACTTGCTCTCGCTGCGGCTTCACACCTTAAGTGCTTAACCTCGCCG |
| | 214801 | CTCCATTCGGAAATCCACGGATCAATGCCTACTTACGGCTCCCCGTGGCT |
| | 214802 | TTTTACGGTTGAGCCGCAAACTTTCACAACTGACTTAACAACCCGCCTAC |
| | 214803 | CGGTTTAGGCTCTTCCGCGTTCGCTCGCCGCTACTTACGGAATCGAGTTT |
| | 214804 | CTTCACTATATACTCTAGTACAGGAATATCAACCTGTTGGCCATCGGATA |
| | 214805 | TGTTTCAGTTCACTGCGTCTTCCTTCTCATAACCTTAACAGTTATGGATA |
| | 214806 | GACGGAGCTTATCCCCCGCCGACTCACTGCCGGGATACGCGTCACGGGTA |
| | 214807 | CCGAACTGTCTCACGACGTTCTGAACCCAGCTCGCGTACCGCTTTAATGG |
| | 214808 | GACGGTGACAAGCCGGTACCAGAATATCAACTGGTTACCCATCGACTACG |
| | 214809 | GATGCGCATTCGGAGTTTGTCAAGACTTGATAGGCGGTGAAGCCCTCGCA |
| | 214810 | TAGGTGAGCCGTTACCCCACCTACTAGCTAATCCCATCTGGGCACATCCG |
| | 214811 | TGGTCCCCGCTCATTCCATCAAGGTTTCTCGTGTCTCGATGTACTCTGGA |
| | 214812 | ATGCTCCCCTACCGATACTTTTTAATGCTATCCCGCGCCTTCGGTACCTG |
| | 214813 | TTACCTTTACTTCAACCTGACCATGGGTAGGTCACCCGGTTTCGGGTCGA |
| | 214814 | GTAGTATTTAGCCTTGGAGGATGGTCCCTCCTGCTTCCCACAGGGTTTCA |
| | 214815 | GATTTCCAACCATTCTGAGGGAACCTTTGGGCGCCTCCGTTACCTTTTAG |
| | 214816 | ATCCCTTCCGGGCTTGGCTACTCGGCCGTAGACTTGGCAGTCTAACCGAT |
| | 214817 | GATGCGCATTCGGAGTTTGTCAAGACTTGATAGGCGGTGAAGCCCTCGCA |
| | 214818 | GTAATCGCCTTGGTGGGCCATTACCCCACCAACAAGCTGATAGGCCGCAG |
| | 214819 | ACCCTCAGGTCATCCAGAAGCTTTTCAACGCTTATTGGTTCGGTCCTCCA |
| | 214820 | AGCTCCATGGGGTCTTTCCGTCTAGTTGCGGGTAACCTGCATTTTCACAG |
| | 214821 | CGTGGGGATTAAGTTTAGCGGATTTTCTCGGGAGTATGATTACGTGCGCT |
| | 214822 | TATTTTGGGACCTTAACTGGCGGTCTGGGCTGTTTCCCTCTTGACCATGG |
| | 214823 | TAACCTTGCACGGGATCGTAACTCGCCGGTTCATTCTACAAAAGGCACGC |
| | 214824 | GACGGCCCAGAGACCTGCCTTCGCCATCGGTGTTCTTCCCGATATCTACA |
| | 214825 | TCACACGGGATTCCACGAGTCCCGCGCTACTTGGGAGACACGATCCGGAG |
| | 214826 | AGTATTTAGCCTTGGAGGATGGTCCCCCCATATTCAGACAGGATACCACG |
| | 214827 | TTTGGCCTCTTCCGCGTTCGCTCGCCACTACTAGCGGAATCTCGGTTGAT |
| | 214828 | CTGCTTCCAAGCCAACATCCTAGCTGTCTTAGCAGTCAGACTTCGTTAGT |
| | 214829 | CTGGGGCTTCAATTCACACCTTCGCTTACGCTAAGCGCTCCTCTTAACCT |
| | 214830 | GTTTGGGCTTCTCCCCTTTCGCTCGCCGCTACTCAGGGAATCACTGTTGT |
| | 214831 | ACAATCCACACCGAATGCCAATACCAAGGTATAGTAAAGGTCCCGGGGTC |
| | 214832 | CAGGGTAGCTTTTATCCGTTGAGCGATGGCCCTTCCATACGGTACCACCG |
| | 214833 | ATAGGCGGTGAAGCCCTCTTGACCTATCGGTCGCTCTACCTCTCACGGTG |
| | 214834 | GCCATGCAGATTCTCACTGCATTCGCGCTACTCATTCCGGCATTCTCACT |
| | 214835 | CGGTACGCCGCCGGTACGGGAATATCCACCCGTTCATCCATTCGACTACG |
| | 214836 | GCACTCCACAGCTCCTTCCGGTACTGCTTCTTCGCGTTAAGAATGCTCCT |
| | 214837 | CGTTCACTCTTCCTTGGCTCCTACCTATCCTGTACATGTGTAACAGATAC |
| | 214838 | CCCCTGACCTGATTCAAGGCCACAGGTTAGAATTTCAGCACTTCAAGAGT |
| | 214839 | CTACCCAGCAATGCCTTTGGCAAGACAACTGGTACACCAGCGGTAAGTCC |
| | 214840 | CCAGCACCGGGCAGGCGTCACCCCCTATACTTCATCTTACGATTTCGCAG |
| | 214841 | ATTCCTCACTGCTGCCTCCCGTAGGAGTTTGGACCGTGTCTCAGTCCCAA |
| | 214842 | CTACGAGACTCAAGCTTGCCAGTATCAGATGCAGTTCCCAGGTTGAGCCC |
| | 214843 | CTCTCAACGATGACGTCTCCTCTTAACCTTCCAGCACCGGGCAGGTGTCA |
| | 214844 | ATTACCGCGGCTGCTGGCACGGAGTTAGCCGGTGCTTCTTCTGCGGGTAA |
| | 214845 | GCGATGGACTTTCACACCGGACGCGACGAGCCGCCTACGAGCCCTTTACG |
| | 214846 | CCCACACCGGATATGGACCGAACTGTCTCACGACGTTCTGAACCCAGCTC |
| | 214847 | GAATGAATGGCTGCTTCTGAGCCAACATCCTAGTTGTCTTAGAGATCCCA |
| | 214848 | TCCCCGGAGTACCTTTTATCCTTTGAGCGATGTCCCTTCCATACGGAAAC |
| | 214849 | GTAAAGCCACCTTATACCCTTGCATTCTACAGGAGATTTCTGACCTCCTT |
| | 214850 | TCCGCCTGCGCACCCTTTAAACCCAATAAATCCGGATAACGCTCGTATCC |
| | 214851 | AGGAAGTATTCAGGCTTACCAGGTGGTCCTGGCAGATTCACACACGATTC |
| | 214852 | GTGTAGGATTCTCACCTACATCTCGCTACTCACACCGGCATTCTCACTTC |
| | 214853 | GAACTGAGACCGGTTTTCAGGGATCCGCTCCATGTCGCCATGTCGCATCC |
| | 214854 | TTCCTGAAGTTGATTCTTCGGGTTAGACAGCCAAACTTCTCAGGGTGGTA |
| | 214855 | CGGTACTGGTACGCTATCGGTCAGACAGGTATGCTTAGACTTACGCCACG |
| | 214856 | GTTTCCCCTCGACTTGCATGTGTTAAGCCTGTAGCTAGCGTTCATCCTGA |
| | 214857 | CGAAGTTACGGGGTCATTTTGCCGAGTTCCTTGACAATGCTTCTCCCGCC |
| | 214858 | CTTGGGAATGATCAGCCTGTTATCCCCGGGGTACCTTTTATCCGTTGAGC |
| | 214859 | GTCTATAAGTACTTCGATTTTTGCAAGTCCGAACCCCGAACGTCCGTAGA |
| | 214860 | CACCTTTCCTTCACAGTACTGGTTCACTATCGGTCTCTCGGGAGTATTTA |
| | 214861 | CCGGGAATTCCAGTCTCCCCTACCGCACTCCAGCCCGCCCGTACCCGGCG |
| | 214862 | ACAGCTTTTCTCGCCATCTTCCATCTCGGACTTCGGTACTAATTTCCCTC |
| | 214863 | TCTTTCGGCGAGGGGGGTTCCCGCCCCCTTTATCGTTACTTATACCTACA |
| | 214864 | TGTATGCGCCATTGTAGCACGTGTGTAGCCCTGGTCGTAAGGGCCATGAT |
| | 214865 | CTTTCGTCTCTGATCGAGTTGTCACTCTCGCAGTCAGGCACCCTTCTGCC |
| | 214866 | GATACTACAATTTCACTGAGCTCTTGGTTGAGACAGCGTCCGGATCATTA |
| | 214867 | GATGTTTCAGTTCAGGCGGTTCCCTCAATACACCTATTTTAAATTTCAGT |
| | 214868 | AAAAAAAAACAAAAAAAAAAACCCTCCCCCCCCCCCCTTCCCCTCCGCGG |
| | 214869 | GCCCTGTTAAGACTTGGTATCCCTTCGGCTCCGCACCTTAAGTGCTTAAC |
| | 214870 | ACCACGAATTCCGCCTGCCTCAACTGCACTCAAGATATCCAGTATCAACT |
| | 214871 | GAGTTTTTCACACTGTGCCATGCAGCACTGTGCGCTTATGCGGTATTAGC |
| | 214872 | TGCCTAGTTCCTTAACCATGAATCTCTCAACGCCTCAGTATGTTCTACCC |
| | 214873 | GGTGTGTACAAGGCCCGGGAACGTATTCACCGCGCCGTGGCTGATGCGCG |
| | 214874 | TTCGCCACCGGTATTCCTCCAGATCTCTACGCATTTCACCGCTACACCTG |
| | 214875 | CGCTTAACGCGTTAGCTCCGACACGGAACACGTGGAACGTGCCCCACATC |
| | 214876 | ACACGAGCCGAAACCCGTGTCTCTCAGACTCCCACCTATCCTGTGCATCA |
| | 214877 | ACTCGATTTCTCTTCGGCTCCACACCTTAAGTGCTTAACCTTGCCGGCAC |
| | 214878 | TGAACCCGCCCCGAAGGGAAACGCCATCTCTGGCGTCGTCGGGAACATGT |

### DESCRIPTION OF THE EMBODIMENTS

### I. Target and Off-Target Nucleic Acids

Described herein are methods for enriching viral molecules from a nucleic acid sample. In some embodiments, the viral molecules are viral RNA molecules. In some embodiments, the viral molecules are genomic viral DNA or RNA molecules. In some embodiments, solid supports can be prepared for enriching desired library fragments or depleting unwanted library fragments, wherein oligonucleotides are immobilized to the solid support. In some embodiments, the solid support is a flowcell.

Also disclosed herein are compositions comprising a probe set comprising at least two DNA probes complementary to at least one target viral nucleic acid molecules in a nucleic acid sample.

Disclosed herein are also kits for depleting or enriching libraries. In some embodiments, the kit comprises a probe compositions disclosed herein and instructions for using the probe set. Such a kit may further comprise reagents for preparing a cDNA library from RNA, such as reagents for a stranded method of cDNA preparation from a sample comprising RNA, as described below.

### A. Viral Targets

Public health officials need to be able to detect viral pathogens in a variety of environmental samples to detect disease outbreaks in a population and measure the intensity of disease outbreaks. Thus, this approach may be used to detect a variety of viral pathogens. In some embodiments, at least one viral molecule is from a virus listed in Table 1.

TABLE 1
Viral Targets

| | | | | |
|---|---|---|---|---|
| adenovirus | Aichivirus | Chapare | chikungunya | enterovirus |
| coxsackievirus | Crimean-Congo | Dengue virus | eastern equine | Guanarito virus |
| | haemorrhagic | | encephalitis | |
| | fever virus | | virus | |
| Dobrava virus | Saaremaa virus | Puumala virus | Tula virus | Hantaan virus |
| Seoul virus | Anjozorobe | Anjozorobe | Sangassou | |
| | hantavirus | hantavirus | virus | |
| Andes virus | Bermejo virus | Lechiguanas | Rio Mamore | choclo virus |
| | | virus | virus | |
| Maciel virus | Laguna Negra | Araraquara | Castelo dos | Juquitiba virus |
| | | virus | Sonhos virus | |
| bayou virus | Black Creek | sin nombre | orthohantavirus | Monongahela |
| | Canal virus | virus | | hantavirus |
| Hendra virus | hepatitis A | hepatitis B | hepatitis C | human |
| | virus | virus | virus | immunodeficiency |
| | | | | virus 1 |
| human | human | influenza A | influenza B | Japanese |
| immunodeficiency | metapneumovirus | virus | virus | encephalitis |
| virus 2 | | | | virus |
| Lassa virus | Mopeia Lassa | Lujo virus | Machupo virus | Marburg virus |
| | virus | | | |
| Ebola virus | monkeypox | Nipah virus | norovirus | human |
| | virus | | | papillomavirus |
| parainfluenza | parechovirus | Merkel cell | KI | polyomavirus |
| | | polyomavirus | polyomavirus | |
| | | | Stockholm 60 | |
| rhinovirus A | rhinovirus B, | rhinovirus C | Rift Valley | rotavirus A |
| | | | fever | |
| rotavirus B | rotavirus C | rotavirus H | respiratory | Sabia virus |
| | | | syncytial virus | |
| salivirus | sapovirus | SARS | Middle East | human |
| | | coronavirus | respiratory | coronavirus |
| | | | syndrome- | |
| | | | related | |
| | | | coronavirus | |
| tick-borne | Kyasanur forest | Omsk | torque teno | variola virus |
| encephalitis virus | disease virus | hemorrhagic | virus | |
| | | fever virus | | |
| Venezuelan equine | West Nile virus | western equine | yellow fever | Zika virus, |
| encephalitis | | encephalomyelitis | virus | parvovirus |
| virus | | virus | | |
| rubella virus | | | | |

In some embodiments, at least one viral molecule is selected from Adeno-associated virus 2 (AAV2), Aichi virus 1 (AiV-A1), Alkhumra hemorrhagic fever virus (AHFV), Andes virus (ANDV), Anjozorobe virus (ANJV), Araucaria virus, Australian bat lyssavirus (ABLV), Bayou virus (BAYV), BK polyomavirus (BKPyV), Black Creek Canal virus (BCCV), Bombali virus (BOMV), Bourbon virus (BRBV), Bundibugyo virus (BDBV), Cache Valley virus (CVV), California encephalitis virus (CEV), Cedar virus (CedV), Chapare virus (CHAPV), Chikungunya virus (CHIKV), Choclo virus (CHOV), Colorado tick fever virus (CTFV), Crimean-Congo hemorrhagic fever virus (CCHFV), Crimean-Congo hemorrhagic fever virus 2 (CCHFV-2), Dengue virus (DENV), Dobrava-Belgrade virus (DOBV), Duvenhage virus (DUVV), Eastern equine encephalitis virus (EEEV), Ebola virus (EBOV), Enterovirus A, Enterovirus B, Enterovirus C, Enterovirus D, Epstein-Barr virus (EBV), European bat lyssavirus (EBLV), Ghana virus (GhV), Guanarito virus (GTOV), Hantaan virus (HTNV), Heartland virus (HRTV), Hendra virus (HeV), Henipavirus unclassified, Hepatitis A virus (HAV), Hepatitis B virus (HBV), Hepatitis C virus (HCV), Hepatitis D virus (HDV), Hepatitis E virus (HEV), Herpes simplex virus 1 (HSV1), Herpes simplex virus 2 (HSV2), Human adenovirus A, Human adenovirus B, Human adenovirus C, Human adenovirus D, Human adenovirus E, Human adenovirus F, Human adenovirus G, Human bocavirus (HBOV), Human coronavirus 229E (HCoV_229E), Human coronavirus HKU1 (HCOV_HKU1), Human coronavirus NL63 (HCoV_NL63), Human coronavirus OC43 (HCoV_OC43), Human cytomegalovirus (HCMV), Human immunodeficiency virus 1 (HIV-1), Human immunodeficiency virus 2 (HIV-2), Human metapneumovirus (HMPV), Human papillomavirus 11 (HPV11), Human papillomavirus 16 (HPV16; high-risk), Human papillomavirus 18 (HPV18; high-risk), Human papillomavirus 26 (HPV26), Human papillomavirus 31 (HPV31; high-risk), Human papillomavirus 33 (HPV33; high-risk), Human papillomavirus 35 (HPV35; high-risk), Human papillomavirus 39 (HPV39; high-risk), Human papillomavirus 40 (HPV40), Human papillomavirus 42 (HPV42), Human papillomavirus 43 (HPV43), Human papillomavirus 44 (HPV44), Human papillomavirus 45 (HPV45; high-risk), Human papillomavirus 51 (HPV51; high-risk), Human papillomavirus 52 (HPV52; high-risk), Human papillomavirus 53 (HPV53), Human papillomavirus 54 (HPV54), Human papillomavirus 56 (HPV56; high-risk), Human papillomavirus 58 (HPV58; high-risk), Human papillomavirus 59 (HPV59; high-risk), Human papillomavirus 6 (HPV6), Human papillomavirus 61 (HPV61), Human papillomavirus 66 (HPV66; high-risk), Human papillomavirus 68 (HPV68; high-risk), Human papillomavirus 69 (HPV69), Human papillomavirus 70 (HPV70), Human papillomavirus 73 (HPV73), Human papillomavirus 82 (HPV82), Human parainfluenza virus 1 (HPIV-1), Human parainfluenza virus 2 (HPIV-2), Human parainfluenza virus 3 (HPIV-3), Human parainfluenza virus 4 (HPIV-4), Human parechovirus (HPeV), Human parvovirus B19 (B19V), Human polyomavirus 6 (HPyV6), Human polyomavirus 7 (HPyV7), Human polyomavirus 9 (HPyV9), Human respiratory syncytial virus A (HRSV-A), Human respiratory syncytial virus B (HRSV-B), Influenza A virus, Influenza B virus, Influenza C virus, Isla Vista virus, Itapua virus, Jamestown Canyon virus (JCV), Japanese encephalitis virus (JEV), JC polyomavirus (JCPyV), Junin virus (JUNV), Juquitiba virus, KI polyomavirus (KIPyV), Kyasanur Forest disease virus (KFDV), La Crosse virus (LACV), Lagos bat virus (LBV), Laguna Negra virus (LANV), Langya virus, Lassa virus (LASV), LI polyomavirus (LIPyV), Lloviu virus (LLOV), Lujo virus (LUJV), Luxi virus (LUXV), Lymphocytic choriomeningitis virus (LCMV), Machupo virus (MACV), Mamastrovirus 1 (MAstV1), Mamastrovirus 6 (MAstV6), Mamastrovirus 8 (MAstV8), Mamastrovirus 9 (MAstV9), Maporal virus (MAPV), Marburg virus (MARV), Mayaro virus (MAYV), Measles virus (MV), Menangle virus (MenV), Merkel cell polyomavirus (MCPyV), Middle East respiratory syndrome-related coronavirus (MERS-COV), Mojiang virus (MojV), Mokola virus (MOKV), Monkeypox virus (MPV), Monongahela hantavirus, Muleshoe virus, Mumps virus (MuV), Murray Valley encephalitis virus (MVEV), MW polyomavirus (MWPyV), New Jersey polyomavirus (NJPyV), Nipah virus (NiV), Norovirus, Omsk hemorrhagic fever virus (OHFV), Onyong-nyong virus (ONNV), Oropouche virus (OROV), Paranoa virus, Powassan virus (POWV), Punta Toro virus (PTV), Puumala virus (PUUV), Rabies virus (RABV), Ravn virus (RAVV), Reston virus (RESTV), Rhinovirus A (RV-A), Rhinovirus B (RV-B), Rhinovirus C (RV-C), Rift Valley fever virus (RVFV), Ross River virus (RRV), Rotavirus A (RVA), Rotavirus B (RVB), Rotavirus C (RVC), Rubella virus (RuV), Sabia virus (SBAV), Salivirus A (SaV-A), Sandfly fever Sicilian virus (SFCV), Sangassou virus (SANGV), Sapovirus, Semliki Forest virus (SFV), Seoul virus (SEOV), Severe acute respiratory syndrome coronavirus (SARS-COV), Severe acute respiratory syndrome coronavirus 2 (SARS-COV-2), Severe fever with thrombocytopenia syndrome virus (SFTSV), Simian virus 40 (SV40), Sin nombre virus (SNV), Sindbis virus (SINV), Snowshoe hare virus (SSHV), Sosuga virus (SoRV), St. Louis encephalitis virus (SLEV), STL polyomavirus (STLPyV), Sudan virus (SUDV), Tacheng tick virus 2 (TcTV-2), Tahyna virus (TAHV), Tai Forest virus (TAFV), Tick-borne encephalitis virus (TBEV), Torque teno virus (TTV), Toscana virus (TOSV), Trichodysplasia spinulosa-associated polyomavirus (TSPyV), Tula virus (TULV), Usutu virus (USUV), Varicella-zoster virus (VZV), Variola virus (VARV), Venezuelan equine encephalitis virus (VEEV), West Nile virus (WNV), Western equine encephalitis virus (WEEV), WU polyomavirus (WUPyV), Yellow fever virus (YFV), and Zika virus (ZIKV).

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

In some embodiments, the present methods decrease library preparation costs and hands-on-time, as compared to prior art methods of enrichment, followed by library preparation.

As used herein, “desired RNA” or “a desired RNA sequence” refers to any RNA that a user wants to analyze. As used herein, a desired RNA includes the complement of a desired RNA sequence. Desired RNA may be RNA from which a user would like to collect sequencing data, after cDNA and library preparation. In some instances, the desired RNA is mRNA (or messenger RNA). In some instances, the desired RNA is a portion of the mRNA in a sample. For example, a user may want to analyze RNA transcribed from cancer-related genes, and thus this is the desired RNA.

As used herein, “desired library fragments” refers to library fragments prepared from cDNA prepared from desired RNA.

In some embodiments, the desired RNA sequence is sequence from a virus listed in Table 1.

### B. Off Target RNA

Also described herein are methods for depleting off-target RNA molecules from a nucleic acid sample. Samples comprising RNA often have a high abundance of RNA that is not of interest to the user. For example, ribosomal RNA (rRNA) typically comprises most of the RNA molecules in total RNA (approximately 80%-95%). One challenge in RNA sequencing for gene expression analysis is that following RNA extraction most of the extracted material is dominated by a small number of highly abundant transcripts, such as the non-coding ribosomal ribonucleic acids (rRNAs). In a total RNA sample from human blood, globin messenger RNAs (mRNAs) can be present at a dominating level. Accordingly, sequencing RNA transcripts (RNA-Seq) is often inefficient and cost prohibitive for many users and applications. There is a need to deplete abundant transcripts, such as rRNAs and mRNAs, in a sample prior to RNA sequencing.

As used herein, “off-target RNA,” “an off-target RNA sequence”, “unwanted RNA,” or “an unwanted RNA sequence” refers to any RNA that a user does not wish to analyze. As used herein, an unwanted RNA includes the complement of an unwanted RNA sequence. When RNA is converted into cDNA and this cDNA is prepared into a library, a user would sequence library fragments that were prepared from all RNA transcripts in the absence of depletion. Methods described herein for depleting library fragments prepared from unwanted RNA can thus save the user time and consumables related to sequencing and analyzing sequencing data prepared from unwanted RNA. In some embodiments, off-target RNA relates to small non-coding RNA (sncRNA). In some embodiments, the off-target RNA comprises sncRNA with MALAT 1. In some embodiments, off-target RNA comprises at least one small noncoding RNA chosen from RN7SK, RN7SL1, RN7SL2, RN7SL5P, RPPH1, SNORD3A. In some embodiments the off-target RNA is not MALAT1.Small noncoding RNAs are highly abundant as reads during the sequencing process and can lead to noise when analyzing sequencing data. MALAT1 is also highly abundant in the genome. MALAT1 is a highly conserved large, infrequently spliced non-coding RNA which is highly expressed in the nucleus. Trying to remove these reads after sequencing results in wasted sequencing, both in terms of reagents and analysis.

As used herein, “off-target RNA,” “unwanted RNA” or “unwanted RNA sequence” also includes fragments of such RNA. For example, an unwanted RNA may comprise part of the sequence of an unwanted RNA. In some embodiments, unwanted RNA sequence is from human, rat, mouse, or bacteria. In some embodiments, the bacteria are Archaea species, E. Coli, or B. subtilis.

As used herein, “off-target library fragments” or “unwanted library fragments” also includes library fragments prepared from cDNA prepared from unwanted RNA.

Also described herein are compositions comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid, wherein the off-target RNA comprises at least one small noncoding RNA chosen from RN7SK, RN7SL1, RN7SL2, RN7SL5P, RPPH1, and SNORD3A

In some embodiments, the off-target RNA is high-abundance RNA. High-abundance RNA is RNA that is very abundant in many samples and which users do not wish to sequence, but it may or may not be present in a given sample. In some embodiments, the high-abundance RNA sequence is a ribosomal RNA (rRNA) sequence. Exemplary high-abundance RNAs are disclosed in WO2021/127191 and WO 2020/132304.

In some embodiments, the high-abundance RNA sequences are the most abundant RNA sequences determined to be in a sample. In some embodiments, the high-abundance RNA sequences are the most abundant RNA sequences across a plurality of samples even though they may not be the most abundant in a given sample. In some embodiments, a user utilizes a method of determining the most abundant RNA sequences in a sample, as described herein.

In a given sample, the most abundant sequences are the 100 most abundant sequences. In some embodiments, in addition to depleting the 100 most abundant sequences, the method also is capable of depleting the 1,000 most abundant sequences, or the 10,000 most abundant sequences in a sample. In some embodiments, the off-target RNA sequence comprises a sequence with homology of at least 90%, at least 95%, or at least 99% to a most abundant sequence in a sample comprising RNA. In some embodiments, the off-target RNA sequence comprises a sequence with homology of at least 90%, at least 95%, or at least 99% to a most abundant sequence in a sample comprising RNA, wherein the most abundant sequences comprise the 100 most abundant sequences. In some embodiments, homology is measured against the 1,000 most abundant sequences, or the 10,000 most abundant sequences.

In some embodiments, the high-abundance RNA sequences are comprised in RNA known to be highly abundant in a range of samples.

In some embodiments, the off-target RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

In some embodiments, the off-target RNA sequence is 28S, 18S, 5.8S, 5S, 16S, or 12S RNA from humans, or a fragment thereof. In some embodiments, the off-target RNA sequence is rat 16S, rat 28S, mouse 16S, or mouse 28S RNA.

In some embodiments, the off-target RNA sequence is comprised in mRNA related to one or more “housekeeping” genes. For example, a housekeeping gene may be one that is commonly expressed in a sample from a tumor or other oncology-related sample, but that is not implicated in tumor genesis or progression. Housekeeping genes are typically constitutive genes that are required for the maintenance of basal cellular functions that are essential for the existence of a cell, regardless of its specific role in the tissue or organism.

In some embodiments, the off-target RNA sequence is comprised in 23S, 16S, or 5S RNA from Gram-positive or Gram-negative bacteria.

### II. Compositions

Described herein are compositions comprising a probe set comprising at least one DNA probe comprising at least one sequence of SEQ ID NOs: 1-213,280, or its complement.

Also described herein are compositions comprising a probe set comprising at least two DNA probes complementary to at least one target viral nucleic acid molecules in a nucleic acid sample wherein the target viral nucleic comprises at least one virus molecule selected from Table 2.

In some embodiments, the one or more target viral nucleic acids are viral RNA molecules. In some embodiments, the one or more target viral nucleic acids are genomic viral RNA molecules. In some embodiments, the one or more target viral nucleic acids are viral DNA molecules. In some embodiments, the one or more target viral nucleic acids are genomic viral DNA molecules.

In some embodiments, the probe set further comprises at least two DNA probes that each hybridize to at least one target viral molecule selected from Table 1.

In some embodiments, the probe set further comprises at least two DNA probes that each hybridize to at least one target virus molecule selected from Table 2.

TABLE 2
VIRAL TARGETS AND SOURCES

| Virus | Target | Type | Source | Description |
|---|---|---|---|---|
| Adenovirus B3 | Full | type 3, 4, 7 | Consensus | |
| | genome | cause more | | |
| | | outbreaks- 4 | | |
| | | and 7 are live | | |
| | | vaccines for | | |
| | | the army and | | |
| | | are monitored | | |
| | | in env for | | |
| | | shedding | | |
| Adenovirus B7 | Full | | Consensus | |
| | genome | | | |
| Adenovirus E4 | Full | | Consensus | |
| | genome | | | |
| Aichivirus A | Full | | NC_001918.1 | Aichivirus |
| | genome | | | |
| Aichivirus B | Full | | NC_004421.1 | Aichivirus B genomic RNA, |
| | genome | | | complete genome, strain: U-1 |
| Aichivirus C | Full | | Consensus | NC_027054.1, NC_016769.1, |
| | genome | | | NC_011829.1 |
| Astrovirus | Full | | NC_001943.1 | |
| | genome | | | |
| Chapare virus | Segment | | NC_010562.1 | Chapare virus segment S |
| | S | | | |
| Chapare virus | Segment | | NC_010563.1 | Chapare virus segment L |
| | L | | | |
| Chikungunya | Full | | NC_004162.2 | Chikungunya virus |
| | genome | | | |
| Enterovirus | Full | Enterovirus | NC_002058.3 | Poliovirus, complete genome |
| | genome | is a species of | | |
| | | enterovirus. | | |
| | | Its best known | | |
| | | subtype is | | |
| | | poliovirus, the | | |
| | | cause of | | |
| | | poliomyelitis. | | |
| | | [1] There are | | |
| | | three | | |
| | | serotypes of | | |
| | | poliovirus, | | |
| | | PV1, PV2, | | |
| | | and PV3. | | |
| | | Other | | |
| | | subtypes of | | |
| | | Enterovirus | | |
| | | include EV- | | |
| | | C95, EV-C96, | | |
| | | EV-C99, EV- | | |
| | | C102, EV- | | |
| | | C104, EV- | | |
| | | C105, EV- | | |
| | | C109, EV- | | |
| | | C116, EV- | | |
| | | C117, and | | |
| | | EV-C118. | | |
| | | Some non- | | |
| | | polio types of | | |
| | | Enterovirus | | |
| | | have been | | |
| | | associated | | |
| | | with the polio- | | |
| | | like condition | | |
| | | AFP (acute | | |
| | | flaccid | | |
| | | paralysis), | | |
| | | including 2 | | |
| | | isolates of | | |
| | | EV-C95 from | | |
| | | Chad. | | |
| Enterovirus A | Full | | NC_001612.1 | Human enterovirus A, complete |
| | genome | | | genome |
| Enterovirus B | Full | | NC_001472.1 | Human enterovirus B, complete |
| | genome | | | genome |
| Enterovirus D | Full | | NC_001430.1 | Human enterovirus D, complete |
| | genome | | | genome |
| Coxsackieviruses | Full | Enterovirus | AF499635.1 | Human coxsackievirus A1 strain |
| A1 | genome | | | Tompkins |
| Coxsackieviruses | Full | Enterovirus A | NC_038306.1 | Human coxsackievirus A2 strain |
| A2 | genome | | | Fleetwood |
| Coxsackieviruses | Full | Enterovirus A | AY421761.1 | Human coxsackievirus A3 strain |
| A3 | genome | | | Olson |
| Coxsackieviruses | Full | abolished | Consensus | |
| A4 | genome | | | |
| Coxsackieviruses | Full | Enterovirus A | Consensus | |
| A5 | genome | | | |
| Coxsackieviruses | Full | abolished | Consensus | |
| A6 | genome | | | |
| Coxsackieviruses | Full | Enterovirus A | Consensus | |
| A7 | genome | | | |
| Coxsackieviruses | Full | Enterovirus A | Consensus | |
| A8 | genome | | | |
| Coxsackieviruses | Full | Enterovirus B | Consensus | |
| A9 | genome | | | |
| Coxsackieviruses | Full | Enterovirus A | Consensus | |
| A10 | genome | | | |
| Coxsackieviruses | Full | Enterovirus | Consensus | |
| A11 | genome | | | |
| Coxsackieviruses | Full | Enterovirus A | Consensus | |
| A12 | genome | | | |
| Coxsackieviruses | Full | Enterovirus | Consensus | |
| A13 | genome | | | |
| Coxsackieviruses | Full | Enterovirus A | Consensus | |
| A14 | genome | | | |
| Coxsackieviruses | Full | Enterovirus | AF465512.1 | |
| A15 | genome | | | |
| Coxsackieviruses | Full | Enterovirus A | Consensus | |
| A16 | genome | | | |
| Coxsackieviruses | Full | Enterovirus C | Consensus | |
| A17 | genome | | | |
| Coxsackieviruses | Full | Enterovirus C | Consensus | |
| A18 | genome | | | |
| Coxsackieviruses | Full | Enterovirus C | Consensus | |
| A19 | genome | | | |
| Coxsackieviruses | Full | Enterovirus C | Consensus | |
| A20 | genome | | | |
| Coxsackieviruses | Full | Enterovirus C | Consensus | |
| A21 | genome | | | |
| Coxsackieviruses | Full | Enterovirus C | Consensus | |
| A24 | genome | | | |
| Coxsackieviruses | Full | Enterovirus B | Consensus | |
| B1 | genome | | | |
| Coxsackieviruses | Full | Enterovirus B | Consensus | |
| B2 | genome | | | |
| Coxsackieviruses | Full | Enterovirus B | Consensus | |
| B3 | genome | | | |
| Coxsackieviruses | Full | Enterovirus B | Consensus | |
| B4 | genome | | | |
| Coxsackieviruses | Full | Enterovirus B | Consensus | |
| B5 | genome | | | |
| Coxsackieviruses | Full | Enterovirus B | Consensus | |
| B6 | genome | | | |
| Crimean-congo | Full | hhs Select | NC_005300.2 | Crimean-Congo hemorrhagic |
| haemorrhagic | genome | agent | | fever virus segment M |
| fever virus | | | | |
| Crimean-congo | Full | | NC_005301.3 | Crimean-Congo hemorrhagic |
| haemorrhagic | genome | | | fever virus segment L |
| fever virus | | | | |
| Crimean-congo | Full | | NC_005302.1 | Crimean-Congo hemorrhagic |
| haemorrhagic | genome | | | fever virus segment S |
| fever virus | | | | |
| Dengue | Full | Differentiate 4 | NC_001474.2 | Dengue virus 2 |
| serotype 1, 2 , 3, 4 | genome | serotypes | | |
| Dengue | Full | | NC_001475.2 | Dengue virus 3 |
| serotype 1, 2, 3, 4 | genome | | | |
| Dengue | Full | | NC_001477.1 | Dengue virus 1 |
| serotype 1, 2, 3, 4 | genome | | | |
| Dengue | Full | | NC_002640.1 | Dengue virus 4 |
| serotype 1, 2, 3, 4 | genome | | | |
| Eastern equine | full | | NC_003899.1 | Eastern equine encephalitis virus |
| encephalitis | genome | | | |
| Enterovirus 68 | full | enterovirus | NC_038308.1 | Human enterovirus 68 strain |
| | genome | D68 is a | | Fermon |
| | | serotype of | | |
| | | Enterovirus D | | |
| Enterovirus 69 | full | Enterovirus | AY302560.1 | Enterovirus 69 strain Toluca-1 |
| | genome | B69 is a | | |
| | | serotype of | | |
| | | Enterovirus B | | |
| Enterovirus 70 | full | enterovirus | Consensus | EVD70 |
| | genome | D70 is a | | |
| | | serotype of | | |
| | | Enterovirus D | | |
| Enterovirus 71 | full | Enterovirus | Consensus | EVA71b |
| | genome | A71 is a | | |
| | | serotype of | | |
| | | Enterovirus A | | |
| Enterovirus 75 | full | Enterovirus | Consensus | EVB75 |
| | genome | B75 is a | | |
| | | serotype of | | |
| | | Enterovirus B | | |
| Enterovirus 76 | full | Enterovirus | Consensus | EVA76 |
| | genome | A76 is a | | |
| | | serotype of | | |
| | | Enterovirus A | | |
| Enterovirus 77 | full | Enterovirus | AY843302.1 | Human enterovirus 77 strain |
| | genome | B77 is a | | USA/TX97-10394 |
| | | serotype of | | |
| | | Enterovirus B | | |
| Enterovirus 79 | full | enterovirus | Consensus | EVB79 |
| | genome | B79 is a | | |
| | | below-species | | |
| | | classification | | |
| | | of Enterovirus | | |
| | | B | | |
| Enterovirus 80 | full | Enterovirus | Consensus | EVB80 |
| | genome | B80 is a | | |
| | | serotype of | | |
| | | Enterovirus B | | |
| Enterovirus 81 | full | Enterovirus | Consensus | EVB81 |
| | genome | B81 is a | | |
| | | serotype of | | |
| | | Enterovirus B | | |
| Enterovirus 82 | full | Enterovirus | AY843300.1 | Human enterovirus 82 strain |
| | genome | B82 is a | | USA/CA64-10390 |
| | | serotype of | | |
| | | Enterovirus B | | |
| Enterovirus 83 | full | Enterovirus | Consensus | EVB83 |
| | genome | B83 is a | | |
| | | serotype of | | |
| | | Enterovirus B | | |
| Enterovirus 84 | full | Enterovirus | Consensus | EVB84 |
| | genome | B84 is a | | |
| | | serotype of | | |
| | | Enterovirus B | | |
| Enterovirus 85 | full | Enterovirus | Consensus | EVB85 |
| | genome | B85 is a | | |
| | | serotype of | | |
| | | Enterovirus B | | |
| Enterovirus 86 | full | Enterovirus | AY843304.1 | Human enterovirus 86 strain |
| | genome | B86 is a | | BAN00-10354 |
| | | serotype of | | |
| | | Enterovirus B | | |
| Enterovirus 87 | full | Enterovirus | AY843305.1 | Human enterovirus 87 strain |
| | genome | B87 is a | | BAN01-10396 |
| | | serotype of | | |
| | | Enterovirus B | | |
| Enterovirus 88 | full | Enterovirus | Consensus | EVB88 |
| | genome | B88 is a | | |
| | | serotype of | | |
| | | Enterovirus B | | |
| Enterovirus 89 | full | Enterovirus | KT277550.1 | Enterovirus A89 strain KSYPH- |
| | genome | A89 is a | | TRMH22F/XJ/CHN/2011 |
| | | serotype of | | |
| | | Enterovirus A | | |
| Enterovirus 90 | full | Enterovirus | Consensus | EVA90 |
| | genome | A90 is a | | |
| | | serotype of | | |
| | | Enterovirus A | | |
| Enterovirus 91 | full | Enterovirus | AY697461.1 | Human enterovirus 91 |
| | genome | A91 is a | | polyprotein gene |
| | | serotype of | | |
| | | Enterovirus A | | |
| Enterovirus 100 | full | Enterovirus | DQ902713.1 | Human enterovirus 100 isolate |
| | genome | B100 is a | | BAN2000-10500 |
| | | serotype of | | |
| | | Enterovirus B | | |
| Enterovirus 101 | full | Enterovirus | AY843308.1 | Human enterovirus 101 strain |
| | genome | B101 is a | | CIV03-10361 |
| | | serotype of | | |
| | | Enterovirus B | | |
| Guanarito virus | Full | | NC_005077.1 | Guanarito virus segment S |
| | genome | | | |
| Guanarito virus | Full | | NC_005082.1 | Guanarito virus segment L |
| | genome | | | |
| Dobrava- | Segment | | NC_005235.1 | |
| Belgrade | L | | | |
| Dobrava- | Segment | | NC_005234.1 | Dobrava virus complete M |
| Belgrade | M | | | segment gene for glycoprotein |
| | | | | precursor (G1-G2), strain |
| | | | | DOBV/Ano-Poroia/Af19/1999) |
| Dobrava- | Segment | | NC_005233.1 | Dobrava virus complete S |
| Belgrade | S | | | segment gene for nucleocapsid |
| | | | | protein, strain DOBV/Ano- |
| | | | | Poroia/Af19/1999 |
| Saaremaa | Segment | | AJ410618.2 | Saaremaa virus pol gene for |
| | L | | | polymerase, segment L, strain |
| | | | | Saaremaa-160V, genomic RNA |
| Saaremaa | Segment | | AJ616855.1 | Saaremaa virus, segment M, |
| | M | | | partial M gene for G1G2 |
| | | | | glycoprotein precursor, genomic |
| | | | | RNA |
| Saaremaa | Segment | | AJ616854.1 | Saaremaa virus, segment S, S |
| | S | | | gene for nucleocapsid protein, |
| | | | | complete sequence, genomic |
| | | | | RNA |
| Puumala | Segment | | NC_005225.1 | Puumala virus segment L, |
| | L | | | complete genome |
| Puumala | Segment | | NC_005224.1 | Puumala virus segment S, |
| | S | | | complete sequence |
| Puumala | Segment | | NC_005223.1 | Puumala virus segment M, |
| | M | | | complete sequence |
| Tula | Segment | | NC_005226.1 | Tula virus segment L |
| | L | | | |
| Tula | Segment | | NC_005227.2 | Tula virus segment S |
| | S | | | |
| Tula | Segment | | NC_005228.1 | Tula virus segment M |
| | M | | | |
| Hantaan | Segment | | NC_005222.1 | Hantaan virus segment L, |
| | L | | | complete genome |
| Hantaan | Segment | | NC_005219.1 | Hantaan virus, complete genome |
| | M | | | |
| Hantaan | Segment | | NC_005218.1 | Hantaan virus, complete genome |
| | S | | | |
| Seoul | Segment | | NC_005238.1 | Seoul virus strain Seoul 80-39 |
| | L | | | clone 1 |
| Seoul | Segment | | NC_005236.1 | Seoul virus strain 80-39 segment |
| | S | | | S, complete sequence |
| Seoul | Segment | | NC_005237.1 | Seoul virus segment M, complete |
| | M | | | sequence |
| Thailand | Segment | | NC_034555.1 | Anjozorobe hantavirus strain |
| | S | | | Anjozorobe/Em/MDG/2009/AT |
| | | | | D49 nucleocapsid protein (N) |
| | | | | gene |
| Thailand | Segment | | NC_034556.1 | Anjozorobe hantavirus strain |
| | L | | | Anjozorobe/Em/MDG/2009/AT |
| | | | | D49 RNA-dependent RNA |
| | | | | polymerase gene |
| Thailand | Segment | | NC_034563.1 | Anjozorobe hantavirus strain |
| | M | | | Anjozorobe/Em/MDG/2009/AT |
| | | | | D49 glycoprotein precursor gene |
| Sangassou or | Segment | | NC_034516.1 | Sangassou virus strain SA14 |
| related viruses | M | | | glycoprotein precursor (M) gene |
| Sangassou or | Segment | | NC_034517.1 | Sangassou virus strain SA14 |
| related viruses | L | | | RNA polymerase (L) gene |
| Sangassou or | Segment | | NC_034526.1 | Sangassou virus strain SA14 N |
| related viruses | S | | | protein (S) gene |
| Andes | Segment | | NC_003466.1 | Andes virus segment S |
| | S | | | |
| Andes | Segment | | NC_003467.2 | Andes virus segment M |
| | M | | | |
| Andes | Segment | | NC_003468.2 | Andes virus segment L |
| | L | | | |
| Bermejo | Segment | | AF482713.1 | Bermejo virus strain Oc22531 |
| | S | | | segment S, complete sequence |
| Lechiguanas | Segment | | AF028022.1 | Lechiguanas virus strain |
| | M | | | Of22819 glycoprotein G1 and |
| | | | | G2 precursor, gene, complete cds |
| Lechiguanas | Segment | | AF482714.1 | Lechiguanas virus strain 22819 |
| | S | | | segment S, complete sequence |
| Rio Mamore | Segment | | FJ809772.1 | Rio Mamore virus isolate HTN- |
| | L | | | 007 segment L, complete |
| | | | | sequence |
| Rio Mamore | Segment | | FJ608550.1 | Rio Mamore virus strain HTN- |
| | M | | | 007 segment M, complete |
| | | | | sequence |
| Rio Mamore | Segment | Only partial S | FJ532244.1 | Rio Mamore virus strain HTN- |
| | S | available | | 007 nucleocapsid protein gene, |
| | | | | complete cds |
| Choclo | Segment | | EF397003.1 | Choclo virus strain 588 segment |
| | L | | | L, complete sequence |
| Choclo | Segment | | NC_038374.1 | Choclo virus segment M |
| | M | | | |
| Choclo | Segment | | NC_038373.1 | Choclo virus segment S |
| | S | | | |
| Maciel | Segment | | AF482716.1 | Maciel virus strain 13796 |
| | S | | | segment S, complete sequence |
| Maciel | Segment | | AF028027.1 | Maciel virus strain Bo13796 |
| | M | | | glycoprotein G1 and G2 |
| | | | | precursor, gene, partial cds |
| Laguna Negra | Segment | | NC_038506.1 | Laguna Negra virus glycoprotein |
| | M | | | precursor gene |
| Laguna Negra | Segment | | NC_038505.1 | Laguna Negra virus nucleocapsid |
| | S | | | protein and putative |
| | | | | nonstructural protein genes |
| Araraquara | Segment | | AF307327.1 | Araraquara virus medium RNA |
| | M | | | segment, G1/G2 glycoprotein |
| | | | | precursor gene, partial cds |
| Araraquara | Segment | | EF571895.1 | Araraquara-like virus strain |
| | S | | | P5/Cajuru segment S, complete |
| | | | | sequence |
| Castelo dos | Segment | | AF307326.1 | Castelo dos Sonhos virus |
| Sonhos | M | | | medium RNA segment, G1/G2 |
| | | | | glycoprotein precursor gene, |
| | | | | partial cds |
| Castelo dos | Segment | | JX443691.1 | Castelo dos Sonhos-2 virus strain |
| Sonhos | S | | | AN717307/BRA299 |
| | | | | nucleocapsid protein gene, |
| | | | | complete cds |
| Juquitiba | Segment | | KF913849.1 | Juquitiba virus strain LBCE |
| | S | | | 12070 nucleoprotein gene, |
| | | | | complete cds |
| Bayou | Segment | | NC_038298.1 | Bayou virus nucleocapsid protein |
| | L | | | |
| Bayou | Segment | | NC_038299.1 | Bayou virus isolate HV |
| | M | | | F0260003 segment L |
| Bayou | Segment | | NC_038300.1 | Bayou virus glycoprotein |
| | S | | | precursor |
| Black Creek | Segment | | GU997097.1 | Black Creek Canal virus strain |
| Canal | L | | | SPB 9408076 segment L, |
| | | | | complete sequence |
| Black Creek | Segment | | NC_043073.1 | Black Creek Canal virus M |
| Canal | M | | | segment |
| Black Creek | Segment | | NC_043075.1 | Black Creek Canal virus S |
| Canal | S | | | segment sequence |
| Sin Nombre | Segment | | NC_005215.1 | Sin Nombre virus segment M |
| | M | | | |
| Sin Nombre | Segment | | NC_005216.1 | Sin Nombre virus segment S |
| | S | | | |
| Sin Nombre | Segment | | NC_005217.1 | Sin Nombre virus map viral |
| | L | | | genome L segment |
| New York | Segment | | MG717393.1 | Orthohantavirus sp. strain New |
| | L | | | York 1 segment L, complete |
| | | | | sequence |
| New York | Segment | | MG717392.1 | Orthohantavirus sp. strain New |
| | M | | | York 1 segment M, complete |
| | | | | sequence |
| New York | Segment | | MG717391.1 | Orthohantavirus sp. strain New |
| | S | | | York 1 segment S, complete |
| | | | | sequence |
| Monongahela | Segment | | MH539865.1 | Monongahela hantavirus isolate |
| | L | | | USA_PA_1997 segment L, |
| | | | | complete sequence |
| Monongahela | Segment | | MH539866.1 | Monongahela hantavirus isolate |
| | M | | | USA_PA_1997 segment M, |
| | | | | complete sequence |
| Monongahela | Segment | | MH539867.1 | Monongahela hantavirus isolate |
| | S | | | USA_PA_1997 segment S, |
| | | | | complete sequence |
| Hendra | full | HHS Select | NC_001906.3 | Hendra virus, complete genome |
| henipavirus | genome | agents | | |
| Hepatitis A | Full | | NC_001489.1 | Hepatitis A virus, complete |
| | genome | | | genome |
| Hepatitis B | Full | | NC_003977.2 | Hepatitis B virus (strain ayw) |
| | genome | | | genome |
| Hepatitis C | Full | | NC_004102.1 | Hepatitis C virus genotype 1 |
| | genome | | | |
| Hepatitis C | Full | | NC_009823.1 | Hepatitis C virus genotype 2 |
| | genome | | | |
| Hepatitis C | Full | | NC_009824.1 | Hepatitis C virus genotype 3 |
| | genome | | | |
| Hepatitis C | Full | | NC_009825.1 | Hepatitis C virus genotype 4 |
| | genome | | | |
| Hepatitis C | Full | | NC_009826.1 | Hepatitis C virus genotype 5 |
| | genome | | | |
| Hepatitis C | Full | | NC_009827.1 | Hepatitis C virus genotype 6 |
| | genome | | | |
| Hepatitis C | Full | | NC_030791.1 | Hepatitis C virus genotype 7 |
| | genome | | | |
| Hepatitis E | Full | | NC_001434.1 | Hepatitis E virus, complete |
| | genome | | | genome |
| HIV 1 | Full | | NC_001802.1 | Human immunodeficiency virus |
| | genome | | | 1 |
| HIV 2 | Full | | NC_001722.1 | Human immunodeficiency virus |
| | genome | | | 2 |
| Human | Full | | NC_0391991 | Human metapneumovirus isolate |
| Metapneumovirus | genome | | | 00-1 |
| Influenza A | Segment | | NC_007366.1 | Influenza A virus (A/New |
| virus | 4 | | | York/392/2004(H3N2)) |
| Influenza A | Segment | | NC_007367.1 | Influenza A virus (A/New |
| virus | 7 | | | York/392/2004(H3N2)) |
| Influenza A | Segment | | NC_007368.1 | Influenza A virus (A/New |
| virus | 6 | | | York/392/2004(H3N2)) |
| Influenza A | Segment | | NC_007369.1 | Influenza A virus (A/New |
| virus | 5 | | | York/392/2004(H3N2)) |
| Influenza A | Segment | | NC_007370.1 | Influenza A virus (A/New |
| virus | 8 | | | York/392/2004(H3N2)) |
| Influenza A | Segment | | NC_007371.1 | Influenza A virus (A/New |
| virus | 3 | | | York/392/2004(H3N2)) |
| Influenza A | Segment | | NC_007372.1 | Influenza A virus (A/New |
| virus | 2 | | | York/392/2004(H3N2)) |
| Influenza A | Segment | | NC_007373.1 | Influenza A virus (A/New |
| virus | 1 | | | York/392/2004(H3N2)) |
| Influenza A | Segment | | NC_007382.1 | Influenza A virus |
| virus | 6 | | | (A/Korea/426/1968(H2N2)) |
| Influenza A | Segment | | NC_007374.1 | Influenza A virus |
| virus | 4 | | | (A/Korea/426/1968(H2N2)) |
| Influenza A | Segment | | NC_007381.1 | Influenza A virus |
| virus | 5 | | | (A/Korea/426/1968(H2N2)) |
| Influenza A | Segment | | NC_007375.1 | Influenza A virus |
| virus | 2 | | | (A/Korea/426/1968(H2N2)) |
| Influenza A | Segment | | NC_007380.1 | Influenza A virus |
| virus | 8 | | | (A/Korea/426/1968(H2N2)) |
| Influenza A | Segment | | NC_007376.1 | Influenza A virus |
| virus | 3 | | | (A/Korea/426/1968(H2N2)) |
| Influenza A | Segment | | NC_007377.1 | Influenza A virus |
| virus | 7 | | | (A/Korea/426/1968(H2N2)) |
| Influenza A | Segment | | NC_007378.1 | Influenza A virus |
| virus | 1 | | | (A/Korea/426/1968(H2N2)) |
| Influenza A | Segment | | NC_026422.1 | Influenza A virus |
| virus | 1 | | | (A/Shanghai/02/2013(H7N9)) |
| Influenza A | Segment | | NC_026423.1 | Influenza A virus |
| virus | 2 | | | (A/Shanghai/02/2013(H7N9)) |
| Influenza A | Segment | | NC_026424.1 | Influenza A virus |
| virus | 3 | | | (A/Shanghai/02/2013(H7N9)) |
| Influenza A | Segment | | NC_026425.1 | Influenza A virus |
| virus | 4 | | | (A/Shanghai/02/2013(H7N9)) |
| Influenza A | Segment | | NC_026426.1 | Influenza A virus |
| virus | 5 | | | (A/Shanghai/02/2013(H7N9)) |
| Influenza A | Segment | | NC_026429.1 | Influenza A virus |
| virus | 6 | | | (A/Shanghai/02/2013(H7N9)) |
| Influenza A | Segment | | NC_026427.1 | Influenza A virus |
| virus | 7 | | | (A/Shanghai/02/2013(H7N9)) |
| Influenza A | Segment | | NC_026428.1 | Influenza A virus |
| virus | 8 | | | (A/Shanghai/02/2013(H7N9)) |
| Influenza A | Segment | | NC_026436.1 | Influenza A virus |
| virus | 5 | | | (A/California/07/2009(H1N1)) |
| Influenza A | Segment | | NC_026431.1 | Influenza A virus |
| virus | 7 | | | (A/California/07/2009(H1N1)) |
| Influenza A | Segment | | NC_026432.1 | Influenza A virus |
| virus | 8 | | | (A/California/07/2009(H1N1)) |
| Influenza A | Segment | | NC_026433.1 | Influenza A virus |
| virus | 4 | | | (A/California/07/2009(H1N1)) |
| Influenza A | Segment | | NC_026437.1 | Influenza A virus |
| virus | 3 | | | (A/California/07/2009(H1N1)) |
| Influenza A | Segment | | NC_026434.1 | Influenza A virus |
| virus | 6 | | | (A/California/07/2009(H1N1)) |
| Influenza A | Segment | | NC_026435.1 | Influenza A virus |
| virus | 2 | | | (A/California/07/2009(H1N1)) |
| Influenza A | Segment | | NC_026438.1 | Influenza A virus |
| virus | 1 | | | (A/California/07/2009(H1N1)) |
| Influenza A | Segment | | NC_002023.1 | Influenza A virus (A/Puerto |
| virus | 1 | | | Rico/8/1934(H1N1)) |
| Influenza A | Segment | | NC_002021.1 | Influenza A virus (A/Puerto |
| virus | 2 | | | Rico/8/1934(H1N1)) |
| Influenza A | Segment | | NC_002022.1 | Influenza A virus (A/Puerto |
| virus | 3 | | | Rico/8/1934(H1N1)) |
| Influenza A | Segment | | NC_002017.1 | Influenza A virus (A/Puerto |
| virus | 4 | | | Rico/8/1934(H1N1)) |
| Influenza A | Segment | | NC_002019.1 | Influenza A virus (A/Puerto |
| virus | 5 | | | Rico/8/1934(H1N1)) |
| Influenza A | Segment | | NC_002018.1 | Influenza A virus (A/Puerto |
| virus | 6 | | | Rico/8/1934(H1N1)) |
| Influenza A | Segment | | NC_002016.1 | Influenza A virus (A/Puerto |
| virus | 7 | | | Rico/8/1934(H1N1)) |
| Influenza A | Segment | | NC_002020.1 | Influenza A virus (A/Puerto |
| virus | 8 | | | Rico/8/1934(H1N1)) |
| Influenza A | | | NC_007357.1 | Influenza A virus |
| virus | | | | (A/goose/Guangdong/1/1996(H5N1)) |
| | | | | polymerase (PB1) and PB1- |
| | | | | F2 protein (PB1-F2) genes |
| Influenza A | Segment | | NC_007358.1 | Influenza A virus |
| virus | 2 | | | (A/goose/Guangdong/1/1996(H5N1)) |
| | | | | polymerase (PB1) and PB1- |
| | | | | F2 protein (PB1-F2) genes |
| Influenza A | | | NC_007359.1 | Influenza A virus |
| virus | | | | (A/goose/Guangdong/1/1996(H5N1)) |
| | | | | polymerase (PA) and PA-X |
| | | | | protein (PA-X) genes, complete |
| | | | | cds |
| Influenza A | Segment | | NC_007362.1 | Influenza A virus |
| virus | 4 | | | (A/goose/Guangdong/1/1996(H5N1)) |
| | | | | hemagglutinin (HA) gene |
| Influenza A | | | NC_007360.1 | Influenza A virus |
| virus | | | | (A/Goose/Guangdong/1/96(H5N1)) |
| | | | | nucleocapsid protein (NP) |
| | | | | gene |
| Influenza A | | | NC_007361.1 | Influenza A virus |
| virus | | | | (A/Goose/Guangdong/1/96(H5N1)) |
| | | | | neuraminidase (NA) gene |
| Influenza A | Segment | | NC_007363.1 | Influenza A virus |
| virus | 7 | | | (A/goose/Guangdong/1/1996(H5N1)) |
| | | | | segment 7, complete |
| | | | | sequence |
| Influenza A | Segment | | NC_007364.1 | Influenza A virus |
| virus | 8 | | | (A/goose/Guangdong/1/1996(H5N1)) |
| | | | | segment 8 |
| Influenza A | | | NC_004910.1 | Influenza A virus pb2 gene for |
| virus | | | | polymerase Pb2, genomic RNA, |
| | | | | strain A/Hong |
| | | | | Kong/1073/99(H9N2) |
| Influenza A | | | NC_004911.1 | Influenza A virus pbl gene for |
| virus | | | | polymerase Pb1, genomic RNA, |
| | | | | strain A/Hong |
| | | | | Kong/1073/99(H9N2) |
| Influenza A | | | NC_004912.1 | Influenza A virus pa gene for |
| virus | | | | polymerase PA, genomic RNA, |
| | | | | strain A/Hong |
| | | | | Kong/1073/99(H9N2) |
| Influenza A | | | NC_004908.1 | Influenza A virus ha gene for |
| virus | | | | Hemagglutinin, genomic RNA, |
| | | | | strain A/Hong |
| | | | | Kong/1073/99(H9N2) |
| Influenza A | Segment | | NC_004905.2 | Influenza A virus (A/Hong |
| virus | 5 | | | Kong/1073/99(H9N2)) segment |
| | | | | 5 |
| Influenza A | | | NC_004909.1 | Influenza A virus na gene for |
| virus | | | | neuraminidase, genomic RNA, |
| | | | | strain A/Hong |
| | | | | Kong/1073/99(H9N2) |
| Influenza A | Segment | | NC_004907.1 | Influenza A virus (A/Hong |
| virus | 7 | | | Kong/1073/99(H9N2)) segment |
| | | | | 7 |
| Influenza A | Segment | | NC_004906.1 | Influenza A virus (A/Hong |
| virus | 8 | | | Kong/1073/99(H9N2)) segment |
| | | | | 8 |
| Influenza B | RNA 1 | | NC_002205.1 | Influenza B virus (B/Lee/1940) |
| virus | | | | |
| Influenza B | RNA 2 | | NC_002204.1 | Influenza B virus (B/Lee/1940) |
| virus | | | | segment 2 |
| Influenza B | RNA 3 | | NC_002206.1 | Influenza B virus (B/Lee/1940) |
| virus | | | | segment 3 |
| Influenza B | RNA 4 | | NC_002210.1 | Influenza B virus (B/Lee/1940) |
| virus | | | | segment 4 |
| Influenza B | RNA 5 | | NC_002209.1 | Influenza B virus (B/Lee/1940) |
| virus | | | | segment 5 |
| Influenza B | RNA 6 | | NC_002207.1 | Influenza B virus (B/Lee/1940) |
| virus | | | | segment 6 |
| Influenza B | RNA 7 | | NC_002211.1 | Influenza B virus (B/Lee/1940) |
| virus | | | | segment 7 |
| Influenza B | RNA 8 | | NC_002208.1 | Influenza B virus (B/Lee/1940) |
| virus | | | | segment 8 |
| Japanese | full | | NC_001437.1 | Japanese encephalitis virus |
| ecephalitis virus | genome | | | |
| JEV | | | | |
| Junin virus | Full | | NC_005081.1 | Junin virus segment S |
| | genome | | | |
| Junin virus | Full | | NC_005080.1 | Junin virus segment L |
| | genome | | | |
| Lassa fever | Full | hhs Select | NC_004296.1 | Lassa virus segment S |
| virus | genome | agent | | |
| Lassa fever | Full | | NC_004297.1 | Lassa virus segment L |
| virus | genome | | | |
| Mopeia Lassa | Full | | NC_006573.1 | Mopeia Lassa reassortant 29 |
| | genome | | | segment S |
| Mopeia Lassa | Full | | NC_006572.1 | Mopeia Lassa reassortant 29 |
| | genome | | | segment L |
| Lujo virus | Full | hhs Select | NC_012776.1 | Lujo virus segment S |
| | genome | agent | | |
| Lujo virus | Full | | NC_012777.1 | Lujo virus segment L |
| | genome | | | |
| Machupo virus | Full | | NC_005078.1 | Machupo virus segment S |
| | genome | | | |
| Machupo virus | Full | | NC_005079.1 | Machupo virus segment L |
| | genome | | | |
| Marburg virus | Full | | NC_001608.3 | Marburg marburgvirus isolate |
| | genome | | | Marburg virus/H. sapiens- |
| | | | | tc/KEN/1980/Mt. Elgon-Musoke |
| Ebola virus | Full | | NC_002549.1 | Zaire ebolavirus isolate Ebola |
| | genome | | | virus/H. sapiens- |
| | | | | tc/COD/1976/Yambuku- |
| | | | | Mayinga |
| Monkeypox | Full | hhs Select | NC_003310.1 | Monkeypox virus Zaire-96-1-16 |
| virus | genome | agent | | |
| Nipah | Full | HHS Select | NC_002728.1 | Nipah virus |
| | genome | agent | | |
| Norovirus GI | Full | Alignment | NC_044856.1 | |
| | genome | run - low | | |
| | | percentage | | |
| | | identity | | |
| Norovirus GI | Full | | NC_044854.1 | |
| | genome | | | |
| Norovirus GI | Full | | NC_044853.1 | |
| | genome | | | |
| Norovirus GI | Full | | NC_001959.2 | |
| | genome | | | |
| Norovirus GI | Full | | NC_039897.1 | |
| | genome | | | |
| Norovirus GII | Full | Alignment | NC_044932.1 | |
| | genome | run - low | | |
| | | percentage | | |
| | | identity | | |
| Norovirus GII | Full | | NC_039477.1 | |
| | genome | | | |
| Norovirus GII | Full | | NC_040876.1 | |
| | genome | | | |
| Norovirus GII | Full | | NC_039475.1 | |
| | genome | | | |
| Norovirus GII | Full | | NC_039476.1 | |
| | genome | | | |
| Norovirus GII | Full | | NC_044046.1 | |
| | genome | | | |
| Norovirus GII | Full | | NC_044045.1 | |
| | genome | | | |
| Norovirus GII | Full | | NC_029646.1 | |
| | genome | | | |
| Norovirus GII | Full | | NC_029647.1 | |
| | genome | | | |
| Norovirus GIV | Full | | NC_029647.1 | Norovirus GIV |
| | genome | | | |
| HPV16 | Full | | NC_001526.1 | Human papillomavirus type 16 |
| | genome | | | |
| HPV18 | Full | | NC_001357.1 | Human papillomavirus type 18 |
| | genome | | | |
| HPV31 | Full | | HQ537675.1 | Human papillomavirus type 31 |
| | genome | | | isolate IN221709 |
| HPV33 | Full | | HQ537689.1 | Human papillomavirus type 33 |
| | genome | | | isolate Qv22751 |
| HPV35 | Full | | HQ537729.1 | Human papillomavirus type 35 |
| | genome | | | isolate QV29782 |
| HPV39 | Full | | KC470236.1 | Human papillomavirus type 39 |
| | genome | | | isolate Qv29509 |
| HPV45 | Full | | LR861845.1 | Human papillomavirus type 45 |
| | genome | | | isolate LNS2400068_HPV45 |
| HPV51 | Full | | KF436887.1 | Human papillomavirus 51 isolate |
| | genome | | | BF315 |
| HPV52 | Full | | LC270039.1 | Human papillomavirus type 52 |
| | genome | | | DNA isolate: K0485 |
| HPV56 | Full | | EF177176.1 | Human papillomavirus type 56 |
| | genome | | | clone Qv26762 |
| HPV58 | Full | | KY225961.1 | Human papillomavirus 58 isolate |
| | genome | | | ZWE054176 |
| HPV59 | Full | | LR862007.1 | Human papillomavirus type 59 |
| | genome | | | isolate LNS7199256_HPV59 |
| HPV66 | Full | | U31794.1 | Human papillomavirus type 66 |
| | genome | | | |
| HPV68 | Full | | KC470281.1 | Human papillomavirus type 68 |
| | genome | | | isolate Rw826 |
| Parainfluenza 1 | Full | | NC_003461 | Human parainfluenza virus 1 |
| | genome | | | |
| Parainfluenza 2 | Full | | NC_003443.1 | Human rubulavirus 2 |
| | genome | | | |
| Parainfluenza 3 | Full | | NC_001796.2 | Human parainfluenza virus 3 |
| | genome | | | |
| Parainfluenza 4 | Full | | NC_021928.1 | Human parainfluenza virus 4a |
| | genome | | | viral cRNA strain: M-25 |
| Parechovirus | Full | | NC_001897.1 | Human parechovirus |
| | genome | | | |
| Merkel cell | Full | | NC_010277.2 | Merkel cell polyomavirus isolate |
| polyomavirus | genome | | | R17b |
| isolate R17b | | | | |
| KI | Full | | NC_009238.1 | KI polyomavirus Stockholm 60 |
| polyomavirus | genome | | | |
| Stockholm 60 | | | | |
| BK | Full | | NC_001538.1 | BK polyomavirus |
| polyomavirus | genome | | | |
| JC | Full | | NC_001699.1 | JC polyomavirus |
| polyomavirus | genome | | | |
| WU | Full | | EU711054.1 | WU Polyomavirus strain |
| Polyomavirus | genome | | | WU/Wuerzburg/01/03 |
| Human | Full | | NC_014406.1 | Human polyomavirus 6 |
| polyomavirus 6 | genome | | | |
| Human | Full | | NC_014407.1 | Human polyomavirus 7 |
| polyomavirus 7 | genome | | | |
| Human | Full | | NC_015150.1 | Human polyomavirus 9 |
| polyomavirus 9 | genome | | | |
| Trichodysplasia | Full | | NC_014361.1 | Trichodysplasia spinulosa- |
| spinulosa- | genome | | | associated polyomavirus |
| associated | | | | |
| polyomavirus | | | | |
| Rhinovirus A | Full | | NC_038311.1 | Human rhinovirus 1 strain |
| | genome | | | ATCC VR-1559 |
| Rhinovirus B | Full | | NC_038312.1 | Human rhinovirus 3 |
| | genome | | | |
| Rhinovirus C | Full | | NC_009996.1 | Human rhinovirus C |
| | genome | | | |
| Rift valley fever | Full | HHS Select | NC_014395.1 | Rift Valley fever virus segment S |
| | genome | Agent | | |
| Rift valley fever | Full | | NC_014396.1 | Rift Valley fever virus segment |
| | genome | | | M |
| Rift valley fever | Full | | NC_014397.1 | Rift Valley fever virus segment |
| | genome | | | L |
| Rotavirus A | Segment | | NC_011507.2 | Rotavirus A Segment 1 |
| Segment 1 | 1 | | | |
| Rotavirus A | Segment | | NC_011506.2 | Rotavirus A Segment 2 |
| Segment 2 | 2 | | | |
| Rotavirus A | Segment | | NC_011508.2 | Rotavirus A Segment 3 |
| Segment 3 | 3 | | | |
| Rotavirus A | Segment | | NC_011510.2 | Rotavirus A Segment 4 |
| Segment 4 | 4 | | | |
| Rotavirus A | Segment | | NC_011500.2 | Rotavirus A Segment 5 |
| Segment 5 | 5 | | | |
| Rotavirus A | Segment | | NC_011509.2 | Rotavirus A Segment 6 |
| Segment 6 | 6 | | | |
| Rotavirus A | Segment | | NC_011501.2 | Rotavirus A Segment 7 |
| Segment 7 | 7 | | | |
| Rotavirus A | Segment | | NC_011502.2 | Rotavirus A Segment 8 |
| Segment 8 | 8 | | | |
| Rotavirus A | Segment | | NC_011503.2 | Rotavirus A Segment 9 |
| Segment 9 | 9 | | | |
| Rotavirus A | Segment | | NC_011504.2 | Rotavirus A Segment 10 |
| Segment 10 | 10 | | | |
| Rotavirus A | Segment | | NC_011505.2 | Rotavirus A Segment 11 |
| Segment 11 | 11 | | | |
| Rotavirus B | Segment | | NC_021541.1 | Human rotavirus B strain |
| Segment 1 | 1 | | | Bang373 RNA dependent RNA |
| | | | | polymerase (VP1) mRNA |
| Rotavirus B | Segment | | NC_021545.1 | Human rotavirus B strain |
| Segment 2 | 2 | | | Bang373 inner capsid protein |
| | | | | (VP2) gene |
| Rotavirus B | Segment | | NC_021551.1 | Human rotavirus B strain |
| Segment 3 | 3 | | | Bang373 VP3 (VP3) mRNA |
| Rotavirus B | Segment | | NC_021543.1 | Human rotavirus B strain |
| Segment 4 | 4 | | | Bang373 outer capsid protein |
| | | | | (VP4) gene |
| Rotavirus B | Segment | | NC_021546.1 | Human rotavirus B strain |
| Segment 5 | 5 | | | Bang373 nonstructural protein 1- |
| | | | | 1 (NSP1-1), nonstructural |
| | | | | protein 1-2 (NSP1-2), and |
| | | | | nonstructural protein 1-3 (NSP1- |
| | | | | 3) genes |
| Rotavirus B | Segment | | NC_021544.1 | Human rotavirus B strain |
| Segment 6 | 6 | | | Bang373 inner capsid protein |
| | | | | (VP6) gene |
| Rotavirus B | Segment | | NC_021547.1 | Human rotavirus B strain |
| Segment 7 | 7 | | | Bang373 nonstructural protein |
| | | | | (NSP3) gene |
| Rotavirus B | Segment | | NC_021548.1 | Human rotavirus B strain |
| Segment 8 | 8 | | | Bang373 nonstructural protein |
| | | | | (NSP2) gene |
| Rotavirus B | Segment | | NC_021542.1 | Human rotavirus B strain |
| Segment 9 | 9 | | | Bang373 outer capsid protein |
| | | | | (VP7) gene |
| Rotavirus B | Segment | | NC_021550.1 | Human rotavirus B strain |
| Segment 10 | 10 | | | Bang373 nonstructural protein |
| | | | | (NSP4) gene |
| Rotavirus B | Segment | | NC_021549.1 | Human rotavirus B strain |
| Segment 11 | 11 | | | Bang373 nonstructural protein |
| | | | | (NSP5) gene |
| Rotavirus C | Segment | | NC_007547.1 | Rotavirus C Segment 1 |
| Segment 1 | 1 | | | |
| Rotavirus C | Segment | | NC_007546.1 | Rotavirus C Segment 2 |
| Segment 2 | 2 | | | |
| Rotavirus C | Segment | | NC_007572.1 | Rotavirus C Segment 3 |
| Segment 3 | 3 | | | |
| Rotavirus C | Segment | | NC_007574.1 | Rotavirus C Segment 4 |
| Segment 4 | 4 | | | |
| Rotavirus C | Segment | | NC_007570.1 | Rotavirus C Segment 5 |
| Segment 5 | 5 | | | |
| Rotavirus C | Segment | | NC_007543.1 | Rotavirus C Segment 6 |
| Segment 6 | 6 | | | |
| Rotavirus C | Segment | | NC_007544.1 | Rotavirus C Segment 7 |
| Segment 7 | 7 | | | |
| Rotavirus C | Segment | | NC_007571.1 | Rotavirus C Segment 8 |
| Segment 8 | 8 | | | |
| Rotavirus C | Segment | | NC_007545.1 | Rotavirus C Segment 9 |
| Segment 9 | 9 | | | |
| Rotavirus C | Segment | | NC_007569.1 | Rotavirus C Segment 10 |
| Segment 10 | 10 | | | |
| Rotavirus C | Segment | | NC_007573.1 | Rotavirus C Segment 11 |
| Segment 11 | 11 | | | |
| Rotavirus H | Segment | | NC_007548.1 | Adult diarrheal rotavirus strain |
| Segment 1 | 1 | | | J19 |
| Rotavirus H | Segment | | NC_007549.1 | Adult diarrheal rotavirus strain |
| Segment 2 | 2 | | | J19 |
| Rotavirus H | Segment | | NC_007550.1 | Adult diarrheal rotavirus strain |
| Segment 3 | 3 | | | J19 |
| Rotavirus H | Segment | | NC_007551.1 | Adult diarrheal rotavirus strain |
| Segment 4 | 4 | | | J19 |
| Rotavirus H | Segment | | NC_007552.1 | Adult diarrheal rotavirus strain |
| Segment 5 | 5 | | | J19 |
| Rotavirus H | Segment | | NC_007553.1 | Adult diarrheal rotavirus strain |
| Segment 6 | 6 | | | J19 |
| Rotavirus H | Segment | | NC_007554.1 | Adult diarrheal rotavirus strain |
| Segment 7 | 7 | | | J19 |
| Rotavirus H | Segment | | NC_007555.1 | Adult diarrheal rotavirus strain |
| Segment 8 | 8 | | | J19 |
| Rotavirus H | Segment | | NC_007556.1 | Adult diarrheal rotavirus strain |
| Segment 9 | 9 | | | J19 |
| Rotavirus H | Segment | | NC_007557.1 | Adult diarrheal rotavirus strain |
| Segment 10 | 10 | | | J19 |
| Rotavirus H | Segment | | NC_007558.1 | Adult diarrheal rotavirus strain |
| Segment 11 | 11 | | | J19 |
| RSV | Full | | NC_001803.1 | Respiratory syncytial virus |
| | genome | | | |
| Sabia virus | Segment | | NC_006313.1 | Sabia virus segment L |
| segment L | L | | | |
| Sabia virus | Segment | 3366 | NC_006317.1 | Sabia virus segment S |
| segment S | S | | | |
| Salivirus | Full | | NC_025114.1 | Salivirus FHB |
| | genome | | | |
| Sapovirus | Full | | NC_027026.1 | Sapovirus Hu/Nagoya/NGY- |
| | genome | | | 1/2012/JPN genomic RNA |
| SARS-CoV | Full | | NC_004718.3 | SARS coronavirus Tor2 |
| | genome | | | |
| SARS-CoV-2 | Full | Covers VOC, | NC_045512.2 | Severe acute respiratory |
| | genome | including | | syndrome coronavirus 2 isolate |
| | | alpha, beta, | | Wuhan-Hu-1 |
| | | gamma, delta, | | |
| | | Omicron | | |
| | | (BA1 and | | |
| | | BA2) | | |
| MERS-CoV | Full | | NC_019843.3 | Middle East respiratory |
| | genome | | | syndrome-related coronavirus |
| | | | | isolate HCoV-EMC/2012 |
| hCoV-HKU1 | Full | | NC_006577.2 | Human coronavirus HKU1 |
| | genome | | | |
| hCoV-229E | Full | | NC_002645.1 | Human coronavirus 229E |
| | genome | | | |
| hCoV-NL63 | Full | | NC_005831.2 | Human Coronavirus NL63 |
| | genome | | | |
| hCoV-OC43 | Full | | NC_006213.1 | Human coronavirus OC43 strain |
| | genome | | | ATCC VR-759 |
| Tick-borne | Full | | NC_001672.1 | Tick-borne encephalitis virus |
| encephalitis | genome | | | |
| virus | | | | |
| Kyasanur | Full | | NC_039218.1 | Kyasanur forest disease virus |
| Forest disease | genome | | | polyprotein gene |
| Omsk | Full | | NC_005062.1 | Omsk hemorrhagic fever virus |
| hemorrhagic | genome | | | |
| fever virus | | | | |
| Torque Teno | Full | SSDNA | NC_015783.1 | Torque teno virus |
| virus | genome | | | |
| Variola major | Full | hhs select | NC_001611.1 | Variola virus |
| | genome | agent | | |
| Venezuelan | full | hhs select | NC_001449.1 | Venezuelan equine encephalitis |
| equine | genome | agent | | virus |
| encephalitis | | | | |
| virus | | | | |
| West Nile | Full | | NC_001563.2 | West Nile virus lineage 2 |
| | genome | | | |
| West Nile | Full | | NC_009942.1 | West Nile virus lineage 1 |
| | genome | | | |
| Western equine | full | | NC_003908.1 | Western equine |
| encephalitis | genome | | | encephalomyelitis virus |
| Yellow fever | Full | | NC_002031.1 | Yellow fever virus |
| virus | genome | | | |
| Zika | Full | | NC_012532.1 | Zika virus |
| | genome | | | |
| Zika | Full | | NC_035889.1 | Zika virus isolate ZIKV/H. |
| | genome | | | sapiens/Brazil/Natal/2015 |
| Parvovirus | Full | | NC_000883.2 | Human parvovirus B19 |
| | genome | | | |
| Rubella | Full | | NC_001545.2 | Rubella virus |
| | genome | | | |

In some embodiments, the probe set further comprises at least two DNA probes that each hybridize to at least one target virus molecule selected from Adeno-associated virus 2 (AAV2), Aichi virus 1 (AiV-A1), Alkhumra hemorrhagic fever virus (AHFV), Andes virus (ANDV), Anjozorobe virus (ANJV), Araucaria virus, Australian bat lyssavirus (ABLV), Bayou virus (BAYV), BK polyomavirus (BKPyV), Black Creek Canal virus (BCCV), Bombali virus (BOMV), Bourbon virus (BRBV), Bundibugyo virus (BDBV), Cache Valley virus (CVV), California encephalitis virus (CEV), Cedar virus (CedV), Chapare virus (CHAPV), Chikungunya virus (CHIKV), Choclo virus (CHOV), Colorado tick fever virus (CTFV), Crimean-Congo hemorrhagic fever virus (CCHFV), Crimean-Congo hemorrhagic fever virus 2 (CCHFV-2), Dengue virus (DENV), Dobrava-Belgrade virus (DOBV), Duvenhage virus (DUVV), Eastern equine encephalitis virus (EEEV), Ebola virus (EBOV), Enterovirus A, Enterovirus B, Enterovirus C, Enterovirus D, Epstein-Barr virus (EBV), European bat lyssavirus (EBLV), Ghana virus (GhV), Guanarito virus (GTOV), Hantaan virus (HTNV), Heartland virus (HRTV), Hendra virus (HeV), Henipavirus unclassified, Hepatitis A virus (HAV), Hepatitis B virus (HBV), Hepatitis C virus (HCV), Hepatitis D virus (HDV), Hepatitis E virus (HEV), Herpes simplex virus 1 (HSV1), Herpes simplex virus 2 (HSV2), Human adenovirus A, Human adenovirus B, Human adenovirus C, Human adenovirus D, Human adenovirus E, Human adenovirus F, Human adenovirus G, Human bocavirus (HBOV), Human coronavirus 229E (HCoV_229E), Human coronavirus HKU1 (HCOV_HKU1), Human coronavirus NL63 (HCOV_NL63), Human coronavirus OC43 (HCoV_OC43), Human cytomegalovirus (HCMV), Human immunodeficiency virus 1 (HIV-1), Human immunodeficiency virus 2 (HIV-2), Human metapneumovirus (HMPV), Human papillomavirus 11 (HPV11), Human papillomavirus 16 (HPV16; high-risk), Human papillomavirus 18 (HPV18; high-risk), Human papillomavirus 26 (HPV26), Human papillomavirus 31 (HPV31; high-risk), Human papillomavirus 33 (HPV33; high-risk), Human papillomavirus 35 (HPV35; high-risk), Human papillomavirus 39 (HPV39; high-risk), Human papillomavirus 40 (HPV40), Human papillomavirus 42 (HPV42), Human papillomavirus 43 (HPV43), Human papillomavirus 44 (HPV44), Human papillomavirus 45 (HPV45; high-risk), Human papillomavirus 51 (HPV51; high-risk), Human papillomavirus 52 (HPV52; high-risk), Human papillomavirus 53 (HPV53), Human papillomavirus 54 (HPV54), Human papillomavirus 56 (HPV56; high-risk), Human papillomavirus 58 (HPV58; high-risk), Human papillomavirus 59 (HPV59; high-risk), Human papillomavirus 6 (HPV6), Human papillomavirus 61 (HPV61), Human papillomavirus 66 (HPV66; high-risk), Human papillomavirus 68 (HPV68; high-risk), Human papillomavirus 69 (HPV69), Human papillomavirus 70 (HPV70), Human papillomavirus 73 (HPV73), Human papillomavirus 82 (HPV82), Human parainfluenza virus 1 (HPIV-1), Human parainfluenza virus 2 (HPIV-2), Human parainfluenza virus 3 (HPIV-3), Human parainfluenza virus 4 (HPIV-4), Human parechovirus (HPeV), Human parvovirus B19 (B19V), Human polyomavirus 6 (HPyV6), Human polyomavirus 7 (HPyV7), Human polyomavirus 9 (HPyV9), Human respiratory syncytial virus A (HRSV-A), Human respiratory syncytial virus B (HRSV-B), Influenza A virus, Influenza B virus, Influenza C virus, Isla Vista virus, Itapua virus, Jamestown Canyon virus (JCV), Japanese encephalitis virus (JEV), JC polyomavirus (JCPyV), Junin virus (JUNV), Juquitiba virus, KI polyomavirus (KIPyV), Kyasanur Forest disease virus (KFDV), La Crosse virus (LACV), Lagos bat virus (LBV), Laguna Negra virus (LANV), Langya virus, Lassa virus (LASV), LI polyomavirus (LIPyV), Lloviu virus (LLOV), Lujo virus (LUJV), Luxi virus (LUXV), Lymphocytic choriomeningitis virus (LCMV), Machupo virus (MACV), Mamastrovirus 1 (MAstV1), Mamastrovirus 6 (MAstV6), Mamastrovirus 8 (MAstV8), Mamastrovirus 9 (MAstV9), Maporal virus (MAPV), Marburg virus (MARV), Mayaro virus (MAYV), Measles virus (MV), Menangle virus (MenV), Merkel cell polyomavirus (MCPyV), Middle East respiratory syndrome-related coronavirus (MERS-COV), Mojiang virus (MojV), Mokola virus (MOKV), Monkeypox virus (MPV), Monongahela hantavirus, Muleshoe virus, Mumps virus (MuV), Murray Valley encephalitis virus (MVEV), MW polyomavirus (MWPyV), New Jersey polyomavirus (NJPyV), Nipah virus (NiV), Norovirus, Omsk hemorrhagic fever virus (OHFV), Onyong-nyong virus (ONNV), Oropouche virus (OROV), Paranoa virus, Powassan virus (POWV), Punta Toro virus (PTV), Puumala virus (PUUV), Rabies virus (RABV), Ravn virus (RAVV), Reston virus (RESTV), Rhinovirus A (RV-A), Rhinovirus B (RV-B), Rhinovirus C (RV-C), Rift Valley fever virus (RVFV), Ross River virus (RRV), Rotavirus A (RVA), Rotavirus B (RVB), Rotavirus C (RVC), Rubella virus (RuV), Sabia virus (SBAV), Salivirus A (SaV-A), Sandfly fever Sicilian virus (SFCV), Sangassou virus (SANGV), Sapovirus, Semliki Forest virus (SFV), Seoul virus (SEOV), Severe acute respiratory syndrome coronavirus (SARS-COV), Severe acute respiratory syndrome coronavirus 2 (SARS-COV-2), Severe fever with thrombocytopenia syndrome virus (SFTSV), Simian virus 40 (SV40), Sin nombre virus (SNV), Sindbis virus (SINV), Snowshoe hare virus (SSHV), Sosuga virus (SoRV), St. Louis encephalitis virus (SLEV), STL polyomavirus (STLPyV), Sudan virus (SUDV), Tacheng tick virus 2 (TcTV-2), Tahyna virus (TAHV), Tai Forest virus (TAFV), Tick-borne encephalitis virus (TBEV), Torque teno virus (TTV), Toscana virus (TOSV), Trichodysplasia spinulosa-associated polyomavirus (TSPyV), Tula virus (TULV), Usutu virus (USUV), Varicella-zoster virus (VZV), Variola virus (VARV), Venezuelan equine encephalitis virus (VEEV), West Nile virus (WNV), Western equine encephalitis virus (WEEV), WU polyomavirus (WUPyV), Yellow fever virus (YFV), and Zika virus (ZIKV).

Also described herein are compositions comprising a probe set comprising at least one DNA probe comprising at least one sequence of SEQ ID NOs: 28,453-213,182, or its complement. In some embodiments, the composition comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 2000 or more sequences selected from SEQ ID NOs: 1-184,730 or its complement. In some embodiments, the at least one DNA probe comprises 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more, or 184,730 sequences selected from SEQ ID NOs: 1-184,730 or its complement. In some embodiments, the at least one DNA probe comprises 500 or more, 600 or more, 700 or more, 800 or more, 900 or more, 1000 or more, 2000 or more, or 184,828 sequences selected from SEQ ID NOs: 28,453-213,280, or its complement. In some embodiments, the at least one DNA probe comprises 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more sequences selected from SEQ ID NOs: 28,453-213,182; 213,288-214,878 or its complement.

Also described herein are compositions comprising a probe set comprising at least one DNA probe comprising at least one sequence of SEQ ID NOs: 1-28,452, or its complement. In some embodiments, the composition comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 2000 or more sequences selected from SEQ ID NOs: 1-28,452 or its complement. In some embodiments, the at least one DNA probe comprises 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more sequences selected from SEQ ID NOs: 1-28,452 or its complement. In some embodiments, the at least one DNA probe comprises 500 or more, 600 or more, 700 or more, 800 or more, 900 or more, 1000 or more, 2000 or more sequences selected from SEQ ID NOs: 1-28.452; 213,183-213,280 or its complement. In some embodiments, the at least one DNA probe comprises 500 or more, 600 or more, 700 or more, 800 or more, 900 or more, 1000 or more, 2000 or more sequences selected from SEQ ID NOs: 1-28,452; 213,288-214,878 or its complement.

Also described herein are compositions comprising a probe set comprising at least one DNA probe comprising at least one sequence of SEQ ID NOs: 1-213,280, or its complement. In some embodiments, the composition comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 2000 or more, or 213,280 sequences selected from SEQ ID NOs: 1-213,280, or its complement. In some embodiments, the at least one DNA probe comprises 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more, 200000 or more, sequences selected from SEQ ID NOs: 1-213,280, or its complement. In some embodiments, the at least one DNA probe comprises 500 or more, 600 or more, 700 or more, 800 or more, 900 or more, 1000 or more, 2000 or more, or 213,280 sequences selected from SEQ ID NOs: 1-213,280, or its complement.

In some embodiments, the composition comprises at least 5, at least at least 10, at least 50, at least 100, at least 250, at least 500, at least 750, at least 1000, at least 1500, or at least 2000 sequences of SEQ ID NOs: 1-213,280, or its complement. In some embodiments, the composition comprises two or more, five or more, 10 or more, or 25 or more sequences selected from SEQ ID NOs: 1-213,280, or its complement.

In some embodiments the probe set comprises any one or more of SEQ ID NOs: 213,288-214,878, or its complement.

In some embodiments the probe set is biotinylated.

### III. Methods of Use

### A. Methods of Enriching for Viral Nucleic Acids

Described herein are methods of enriching a sample for one or more target viral nucleic acids.

In some embodiments, the present methods decrease library preparation costs and hands-on-time, as compared to prior art methods of enriching for vial nucleic acids, followed by library preparation.

In some embodiments, the method comprises providing any of the compositions described herein, in Section II (Compositions) above. In some embodiments, the method comprises providing a probe set comprising any of the compositions described herein, in Section II (Compositions) above; allowing the probes in the probe set to hybridize to the target viral nucleic acids; and enriching the sample for the one or more target viral nucleic acids by amplifying the target viral nucleic acids and/or separating the target viral nucleic acids from the sample. In some embodiments, the probe set comprises 1 or more, 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more sequences selected from SEQ ID Nos: 28,453-213,182 or its complement. In some embodiments, the probe set comprises 1 or more, 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more sequences selected from SEQ ID Nos: 28,453-213,182 or its complement.

In some embodiments, the probe set comprises 1 or more, 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more sequences selected from SEQ ID Nos: 1-28,452 or its complement. In some embodiments, the probe set comprises 1 or more, 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more sequences selected from SEQ ID Nos: 1-28,452 or its complement.

In some embodiments, the probe set comprises 1 or more, 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more, 1200 or more, 1300 or more, 1400 or more, 1500 or more, 2000 or more, 3000 or more, 3500 or more, 4000 or more, 5000 or more, 10000 or more, 20000 or more, 3000, or more, 40000 or more, 50000 or more, 100000 or more, 200000 or more, sequences selected from SEQ ID NOs: 1-213,280, or its complement.

In some embodiments, the method comprises providing a probe set comprising at least two nucleic acid probes complementary to one or more target viral nucleic acids, wherein the probe set comprises at least two of SEQ ID NOs: 1-28,452 or SEQ ID NOS: 28,453-213,182 or SEQ ID Nos: 213,183-213,280 or SEQ ID NOs: 1-213,280, or the complements of the foregoing; allowing the probes in the probe set to hybridize to the target viral nucleic acids; and enriching the sample for the one or more target viral nucleic acids by amplifying the target viral nucleic acids and/or separating the target viral nucleic acids from the sample.

Also described herein are methods of enriching a sample for one or more target viral nucleic acids. In some embodiments, the present methods detect or enrich for new or unknown viral pathogens or new or unknown strains of viral pathogens. This may include analysis of patient samples. In some embodiments, the present methods detect co-infections with one or more additional pathogens, including viruses or bacteria. In some embodiments, the present methods detect or enrich for specific viral pathogen strains. In some embodiments, the present methods can be used to perform strain typing and/or strain characterization for monitoring viral pathogen evolution and epidemiology (e.g., viral evolution and epidemiology). In some embodiments, the present methods detect or enrich for viral nucleic acids that exhibit resistance. Resistance can include resistance to anti-viral therapies (whether small molecule therapy or other therapies including treatment with antibodies (including antigen-binding fragments thereof or other biologics with CDRs responsible for specific binding), viral entry inhibitors, viral assembly inhibitors, viral DNA and RNA polymerase inhibitors, viral reverse transcriptase inhibitors, viral protease inhibitors, viral integrase inhibitors, and inhibitors of viral shedding. In some embodiments, the present methods are used to identify hospital-associated viral infections. As used herein, a hospital-associated viral infection refers to an infection whose development spread through and/or is favored by a hospital environment, nursing home, rehabilitation facility, group home, residential facility, medical office, clinic, or other clinical settings. This infection is spread to a subject in the clinical setting by a number of means, for example through contaminated equipment, bed linens, or air droplets. In some embodiments, the present methods are used for viral resequencing. In some embodiments, resequencing allows for testing for known mutations or scanning for one or more mutations in a given target region. Such methods may be used in a panel used for detection of and/or typing of viral pathogens (e.g., viruses-of-interest).

In some embodiments, the method comprises providing a probe set comprising at least two nucleic acid probes complementary to one or more target viral nucleic acids, wherein the nucleic acid probes are affixed to a support; capturing one or more target viral nucleic acids on a support; using the one or more captured target viral nucleic acids as a template strand to produce one or more nucleic acid duplexes immobilized on the support, wherein the at least one target viral nucleic acids hybridize to one or more probes in a probe set on the support; contacting a transposase and transposon with the one or more nucleic acid duplexes under conditions wherein the one or more nucleic acid duplexes and transposon composition undergo a transposition reaction to produce one or more tagged nucleic acid duplexes, wherein the transposon composition comprises a double stranded nucleic acid molecule comprising a transferred strand and a non-transferred strand; contacting the one or more tagged nucleic acid duplexes with a nucleic acid modifying enzyme under conditions to extend the 3′ end of the immobilized strand to the 5′ end of the template strand to produce one or more end-extended tagged nucleic acid duplexes; amplifying the one or more end-extended tagged nucleic acid duplexes to produce a plurality of tagged nucleic acid strands; contacting the plurality of tagged nucleic acid strands with a probe set to create an enriched library; and amplifying the enriched library.

A wide variety of solid supports may be used to immobilize oligonucleotides for depleting or enriching as described herein, including those described in WO 2014/108810, which is incorporated in its entirety herein.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

In some embodiments, a flowcell is comprised within an apparatus or device for sequencing nucleic acids, which may be referred to as a sequencer. In some embodiments, a sequence may also comprise reservoirs for collection of samples or tubing (such as for collecting samples in a reservoir of for exiting of waste). In some embodiments, one or more reservoirs are separate from the flowcell and are comprised in the sequencer. In some embodiments, modifications are made to standard sequencers to improve fluidics system recipes and/or hardware for use of reservoirs in the present methods.

As used herein, a “flowcell” may comprise a flowcell-like device that is not intended to be imaged. While standard flowcells used for imaging may be employed in the present methods, flowcells can also be engineered differently than flowcells intended for imaging. In some embodiments, a flowcell may have a high density of immobilized oligonucleotides, wherein imaging infrastructure would have difficulty separating out into different bridge-amplified clusters associated with different immobilized oligonucleotides. In some embodiments, a high density of immobilized oligonucleotides improves hybridization efficiency. In some embodiments, standard clear glass may be used in a flowcell. In other embodiments, hard plastic may be used in the flowcell. Use of glass in a flowcell may allow use of a standard flowcell without further optimization, whereas use of hard plastic may reduce the cost of manufacturing the flowcell and/or improve stability of a flowcell. Depending on the advantages desired, different materials may be used. In some embodiments, immobilized oligonucleotides are embedded in a substrate other than that of a standard flowcell (i.e., embedded in a substrate other than PAZAM) to improve immobilization of oligonucleotides of longer length.

### B. Methods of Supplementing a Probe Set for Use in Enriching for Viral Nucleic Acids

Also described herein are methods of supplementing a probe set for use in enriching for viral nucleic acid molecules from a nucleic acid sample.

In some embodiments, the methods of enriching for viral nucleic acids described herein can be supplemented with or used in conjunction with other enrichment panels. In some embodiments, the method also targets genitourinary pathogens, Antimicrobial Resistance (AMR) markers, respiratory viruses, respiratory pathogens (e.g., viruses, bacteria, fungi, and/or parasites), and/or exonic content. In some embodiments, the method is used with, supplemented with, or used in conjunction with the Urinary Pathogen ID/AMR Panel or Enrichment Kit (UPIP; Illumina). In some embodiments, the method is used with, supplemented with, or used in conjunction with the Virus Surveillance Panel or Enrichment Kit (VSP; Illumina). In some embodiments, the method is used with, supplemented with, or used in conjunction with the Respiratory Pathogen ID/AMR Panel or Enrichment Kit (RPIP; Illumina). In some embodiments, the method is used with, supplemented with, or used in conjunction with the Pan-Coronavirus Panel or Enrichment Kit (Pan-Cov; Illumina). In some embodiments, the method is used with, supplemented with, or used in conjunction with the Respiratory Virus Oligos Panel or Enrichment Kit (RVOP; Illumina). In some embodiments, the method is supplemented with or used in conjunction with the Illumina Exome Panel (Illumina). In some embodiments, the method targets and enriches for coding RNA sequences. In some embodiments, the method is used with the Illumina RNA Prep with Enrichment (Illumina).

Examples of supplemental probe sets that can be readily used in the methods of the present disclosure are described, for example, in U.S. Provisional Application No. 63/250,563, filed Sep. 30, 2021, U.S. Provisional Application No. 63/351,170 filed Jun. 10, 2022, and U.S. Provisional Application No. 63/378,610, filed Oct. 6, 2022.

In some embodiments the method comprises depleting unwanted nucleic acid molecules from a nucleic acid sample.

In some embodiments, the depleting unwanted nucleic acid molecules comprises depleting unwanted cDNA library fragments from a library of cDNA fragments prepared from RNA, wherein the unwanted library fragments comprise those prepared from unwanted RNA sequences, further comprising: preparing a solid support comprising at least one immobilized oligonucleotide, wherein each immobilized oligonucleotide comprises a nucleic acid sequence corresponding to an unwanted RNA sequence or its complement, adding the library of fragments to the solid support and hybridizing the library fragments to at least one immobilized oligonucleotide to allow binding of unwanted library fragments to at least one immobilized oligonucleotide, and collecting library fragments not bound to at least one immobilized oligonucleotide.

In some embodiments, the at least one immobilized oligonucleotide comprises a sequence comprising any one or more of SEQ ID NOs: 213,288-214,878 or its complement.

In some embodiments, a solid support comprises more than one pool of immobilized oligonucleotides on its surface.

For example, a solid support may comprise a first pool of immobilized oligonucleotides for depleting and a second pool of immobilized oligonucleotides for enriching. In some embodiments, one pool of immobilized oligonucleotides may be blocked (such as with complementary nucleic acid sequences) to avoid binding to complementary library fragments during certain steps of methods using the solid support.

In some embodiments, a solid support has two pools of immobilized oligonucleotides on its surface, wherein the first pool comprises immobilized oligonucleotides each comprising an unwanted RNA sequence and the second pool comprises immobilized oligonucleotides each comprising a solid support adapter sequence that can bind to a library adapter comprised in library fragments. In some embodiments, solid support adapter sequences are bound by adapter complements, wherein the adapter complements can be denatured during a method to allow binding of solid support adapter sequences to library adapters in library fragments. Such a solid support can be used for methods of preparing a depleted library and amplifying the depleted library on the same solid support.

In some embodiments, at least one unwanted RNA sequence has at least 90%, at least 95%, or at least 99% homology to a high-abundance RNA sequence in a sample used to prepare the library of fragments. In some embodiments, all unwanted sequences have at least 90%, at least 95%, or at least 99% homology to a high-abundance RNA sequence in a sample used to prepare the library of fragments.

In some embodiments, the depleting unwanted nucleic acid molecules comprises depleting off-target RNA nucleic acid molecules from a nucleic acid sample comprises contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid, wherein the off-target DNA comprises at least one small noncoding RNA chosen from RN7SK, RN7SL1, RN7SL2, RN7SL5P, RPPH1, SNORD3A; contacting the DNA:RNA hybrids with a ribonuclease that degrades the RNA from the DNA:RNA hybrids, thereby degrading the off-target RNA molecules in the nucleic acid sample to form a degraded mixture; separating the degraded RNA from the degraded mixture; sequencing the remaining RNA from the sample; evaluating the remaining RNA sequences for the presence of off-target RNA molecules from the first species, thereby determining gap sequence regions; and supplementing the probe set with additional DNA probes complementary to discontiguous sequences in one or more of the gap sequence regions.

In some embodiments, the probe set comprises any one or more of SEQ ID NOS: 213,288-214,878, or its complement.

In some embodiments, the method further comprises depleting unwanted cDNA library fragments from a library of cDNA fragments prepared from RNA, wherein the unwanted library fragments comprise those prepared from unwanted RNA sequences.

### C. Samples

The present methods are not limited to a specific type of sample comprising viral RNA or DNA, and these methods can be used with libraries prepared from any sample comprising RNA or DNA. Described below are a few exemplary types of samples, wherein sequencing of library fragments prepared from these samples can be improved by enriching or depleting.

In some embodiments, the sample comprises a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof. In some embodiments, samples are from mixed populations of microbes such as microbial populations or viral populations from patients.

In some embodiments the sample is a water sample. In some embodiments, the water sample is a freshwater sample, a wastewater sample, a saline water sample, or a combination thereof. In some embodiments, the sample comprises a wastewater sample. In some embodiments, the sample comprises wastewater from food production, animal husbandry, seasonal surface runoff or other sources.

In some embodiments, the sample may be from a mammal. In some embodiments the sample may be from a human, monkey, bat, dog, cat, horse, goat, sheep, cow, pig, rat and/or mouse. In some instances, reservoirs of microbes (including viruses) in animal populations can serve as samples to predict what diseases or strains of diseases may become human pathogens or to compare sequences in animal reservoirs to sequences of pathogens infecting humans.

In some embodiments, samples may be from a patient. In some embodiments, samples may be from a patient with cancer (i.e., an oncology sample). In some embodiments, samples may be from a patient with a rare disease. In some embodiments, samples may be from a patient with a viral infection. In some embodiments, samples may be from a patient with coronavirus SARS-CoV2 (COVID-19). In some embodiments, the sample may be a tumor sample. In some embodiments, the sample may be a blood sample, a serum sample, and/or a whole blood sample. In some embodiments the sample may be a tissue sample. In some embodiments the sample may be a fecal sample, a urine sample, a mucus sample, a saliva sample, a lymph sample, a vaginal fluid sample, a semen sample, an amniotic sample, and/or a sweat sample.

### D. Library Preparation

Libraries prepared by any method can be used together with the present methods of enriching and/or depleting. In some embodiments, probes are single-stranded to allow for hybridizing and capturing of single-stranded library fragments that are complementary. In some embodiments, specific binding of a single-stranded library fragment to a probe generates a double-stranded oligonucleotide. In some embodiments, the double-stranded oligonucleotide forms a DNA:RNA hybrid. The probe specifically bound to the library fragment may be bound with a high-enough affinity to be recognized for degradation with a ribonuclease. In some embodiments, the off-target RNA molecules are degraded after contacting the sample with a ribonuclease to form a degraded mixture.

As used herein, the term “library” refers to a collection of members. In one embodiment, the library includes a collection of nucleic acid members, for example, a collection of whole genomic, subgenomic fragments, cDNA, cDNA fragments, RNA, RNA fragments, or a combination thereof. In some embodiments, a portion or all library members include a non-target adaptor sequence. The adaptor sequence can be located at one or both ends. The adaptor sequence can be used in, for example, a sequencing method (for example, an NGS method), for amplification, for reverse transcription, or for cloning into a vector.

In some embodiments, this DNA:RNA hybrid-specific cleavage comprises use of RNase H. This methodology is implemented as part of the current Illumina Total RNA Stranded Library Prep workflow and New England Biolabs NEBNext rRNA Depletion Kit and RNA depletion methods as described in U.S. Pat. Nos. 9,745,570 and 9,005,891.

### E. Amplification

In some embodiments, methods described herein comprise one or more amplification step. In some embodiments, library fragments are amplified before being added to a solid support. In some embodiments library fragments are amplified after a method of depleting described herein. In some embodiments, amplifying is by PCR amplification.

As used herein, “amplify,” “amplifying,” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

1. Amplification after Enriching

In some embodiments, collected library fragments are amplified after a method of enriching. In some embodiments, an enriched library is amplified.

In some embodiments, the amplifying is performed with a thermocycler. In some embodiments, the amplifying is by PCR amplification.

As used herein, the term “polymerase chain reaction” (“PCR”) refers to the method as described in U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing, and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

In some embodiments, the amplifying is performed without PCR amplification. In some embodiments, the amplifying does not require a thermocycler. In some embodiments, depleting and amplifying after the depleting is performed in a sequencer.

In some embodiments, the amplifying is performed without a thermocycler. In some embodiments, the amplifying is performed by bridge or cluster amplification.

### F. Sequencing of Enriched Libraries

In some embodiments, a library enriched for target viral sequences library fragments is sequenced.

In some embodiments, sequencing data generated after enriching for target viral sequences is capable of capturing novel viruses with homology to the sequence in the probe set. In some embodiments, sequencing data generated after enriching for target viral sequences is capable of capturing new or unknown viruses (e.g., new or unknown viruses-of-interest). In some embodiments, sequencing data generated after enriching for target viral sequences is capable of capturing co-infections. In some embodiments, sequencing data generated after enriching for target viral sequences is capable of capturing specific viral strains (e.g., specific strains of a virus-of-interest). In some embodiments, sequencing data generated after enriching for target viral sequences is capable of capturing viral nucleic acids that exhibit resistance. In some embodiments, sequencing data generated after enriching for target viral sequences provides unbiased viral pathogen detection. In some embodiments, sequencing data generated after enriching for target viral sequences is capable of capturing viral nucleic acids present in hospital-associated infection management.

Enriched libraries prepared by the present method can be used with any type of RNA sequencing, such as RNA-seq, small RNA sequencing, long non-coding RNA (lncRNA) sequencing, circular RNA (circRNA) sequencing, targeted RNA sequencing, exosomal RNA sequencing, and degradome sequencing.

Enriched libraries can be sequenced according to any suitable sequencing methodology, such as direct sequencing, including sequencing by synthesis, sequencing by ligation, sequencing by hybridization, nanopore sequencing and the like. In some embodiments, the enriched libraries are sequenced on a solid support. In some embodiments, the solid support for sequencing is the same solid support on which the enriching is performed. In some embodiments, the solid support for sequencing is the same solid support upon which amplification occurs after the enriching.

Flowcells provide a convenient solid support for performing sequencing. One or more library fragments (or amplicons produced from library fragments) in such a format can be subjected to an SBS or other detection technique that involves repeated delivery of reagents in cycles. For example, to initiate a first SBS cycle, one or more labeled nucleotides, DNA polymerase, etc., can be flowed into/through a flowcell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flowcell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008); WO 04/018497; WO 91/06678; WO 07/123744; U.S. Pat. Nos. 7,057,026; 7,211,414; 7,315,019; 7,329,492; 7,405,281; and US Pat. Publication No. 2008/0108082.

### G. Whole Genome Sequencing, Amplicon Sequencing, Metagenomic Analysis, and Metatranscriptomic Analysis

In some embodiments, samples are sequenced using whole-genome sequencing and/or amplicon sequencing. Whole genome sequencing refers to sequencing the genome of any organism including viral pathogens (e.g., viruses-of-interest) and host organisms. For example, whole genome sequencing may be performed on a microbial isolate. Transmission dynamics may be evaluated by whole genome sequencing. Whole genome sequencing also provides useful information on strain characterization, resistance detection, and hospital-associated infection management.

In some embodiments, samples are sequenced using amplicon sequencing. The term “amplicon” refers to the resultant mixture of compounds after two or more cycles of the PCR steps of denaturation, annealing and extension. Thus, amplicon sequencing is the sequencing of amplicons and this can provide useful information on variant identification and characterization. In some embodiments, amplicon sequencing encompasses amplification of one or more segments of one or more target sequences, which can be performed by using probes to target and amplify regions of interest, followed by sequencing, such as next-generation sequencing. Amplicon sequencing may be performed on a variety of samples, including patient samples or microbial isolates, and is useful for strain characterization. It is also useful for viral resequencing and resistance detection.

In some embodiments, additional information may be obtained about samples using metagenomic and/or metatranscriptomic analyses. Metagenomic and/or metatranscriptomic analysis may be performed on patient samples and may provide unbiased viral pathogen detection. In some embodiments, metagenomic or metatranscriptomic analyses comprises sequencing the genomes of a plurality of individuals of different species in a given sample. In some embodiments, metagenomic or metatranscriptomic analyses is done without prior knowledge regarding the biological species in the sample, whether they be viral or human. In some embodiments, metagenomic or metatranscriptomic analyses enables determination of which species are present, and their relative abundances. Thus, metagenomic and/or metatranscriptomic analysis may be useful for unknown viral pathogen detection, co-infection detection, resistance detection, and/or strain characterization.

In some embodiments, whole genome sequencing, amplicon sequencing, metgenomic analysis, and/or metatranscriptomic analyses may be used in combination with each other.

### IV. Kits

Described herein is a kit comprising any of the compositions described herein in Section II, Compositions, above.

Disclosed herein are also kits for depleting or enriching libraries. In some embodiments, the kit comprises a solid support disclosed herein and instructions for using the solid support. Such a kit may further comprise reagents for preparing a cDNA library from RNA, such as reagents for a stranded method of cDNA preparation from a sample comprising RNA, as described below.

In some embodiments the kit comprises at least one DNA probe comprising at least one sequence comprising at least one of SEQ ID NOs: 28,453-213,182, or its complement and a buffer. In some embodiments, the kit comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 2000 or more, or 184,730 sequences selected from SEQ ID NOs: 1-184,730, or its complement. In some embodiments, the at least one DNA probe comprises 500 or more, 600 or more, 700 or more, 800 or more, 900 or more, 1000 or more, 2000 or more, or 184,828 sequences selected from SEQ ID NOs: 28,453-213,280, or its complement.

In some embodiments the kit comprises at least one DNA probe comprising at least one sequence comprising at least one of SEQ ID NOs: 1-28,452, or its complement and a buffer. In some embodiments, the kit comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 2000 or more sequences selected from SEQ ID NOs: 184,829-213,280, or its complement. In some embodiments, the at least one DNA probe comprises 500 or more, 600 or more, 700 or more, 800 or more, 900 or more, 1000 or more, 2000 or more sequences selected from SEQ ID NOs: 1-28,452; 213,183-213,280 or its complement.

In some embodiments the kit comprises at least one DNA probe comprising at least one sequence comprising at least one of SEQ ID NOs: 1-213,280, or its complement and a buffer. In some embodiments, the kit comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 2000 or more, or 213,280 sequences selected from SEQ ID NOs: 1-213,280, or its complement. In some embodiments, the at least one DNA probe comprises 500 or more, 600 or more, 700 or more, 800 or more, 900 or more, 1000 or more, 2000 or more, or 213,280 sequences selected from SEQ ID NOs: 1-213,280, or its complement.

In some embodiments, the kit further comprises at least one DNA probe comprising at least one sequence comprising at least one of SEQ ID Nos: 213,288-214,878, or its complement.

In some embodiments, the buffer is a wash buffer and/or an elution buffer.

In some embodiments, the kit further comprises an RNA depletion buffer, a probe depletion buffer, and/or a probe removal buffer.

In some embodiments, the kit further comprises a ribonuclease; a DNase; and RNA purification beads. In some embodiments, the ribonuclease is RNase H.

In some embodiments, the kit comprises a buffer and nucleic acid purification medium. In some embodiments, the buffer is an RNA depletion buffer, a probe depletion buffer, and/or a probe removal buffer.

In some embodiments, the kit comprises a nucleic acid destabilizing chemical. In some embodiments, the nucleic acid destabilizing chemical comprises betaine, DMSO, formamide, glycerol, or a derivative thereof, or a mixture thereof. In some embodiments, the nucleic acid destabilizing chemical comprises formamide.

Throughout this application and claims, the term “and/or” means one or more of the listed elements or a combination of any two or more of the listed elements.

The term “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” is limited to whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

As used herein, the term “each,” when used in reference to a collection of items, is intended to identify an individual term in the collection but does not necessarily refer to every term in the collection unless the context clearly dictates otherwise.

The recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

All headings are for the convenience of the reader and should not be used to limit the meaning of the text that follows the heading, unless so specified.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

Unless defined otherwise, all technical and scientific terms used herein have the same meaning as commonly understood by one of ordinary skill in the art pertinent to the methods and compositions described. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

### EXAMPLES

### Example 1. Preparation of Probes to Improve Enrichment of Viruses of Interest in Wastewater Samples

### A. Probe Design

Probes were designed that would bind to viruses present in wastewater and known to cause human diseases (i.e., viruses-of-interest).

For most viral species, the RefSeq reference sequences were used. RefSeq is an NCBI Reference Sequence Database. Where no RefSeq genome was available, and few sequences were available in the NCBI database, just one of these accessions was chosen. Where many options were available (generally >3-5) all sequences were aligned, and a consensus sequence was used for the design. See Table 2.

Probes were designed by a proprietary algorithm for enrichment probes running on a Linux server. The weighting for spacing and probe scoring variables were set to 6 and 2 respectively. Probe spacing was set to ‘adjacent’, or 80 bp center to center. After the initial panel was submitted to manufacturing, it was determined that there were some strains of Monkeypox that contained additional sequence not captured in the initial panel. Additional probes were designed to supplement these gaps.

### B. Mitigation of Poly G Sequences

Poly G sequences pose manufacturing problems for enrichment probes and can often result in a failure or premature termination of the oligonucleotide. To mitigate this in the current probe pool, the pool of designed probes was scrutinized and every probe with a run of 4 Gs or more was flagged. In addition, the complete list of candidate probes (outputted by a proprietary algorithm) was scrutinized, and any probe candidates with a run of 4 or more Gs was evaluated for deletion from the list. Finally, an overlap was run on the flagged probes, and they were replaced by a probe candidate which had the greatest amount of overlap with the original. If no probe from the candidate list (not containing >3 Gs) was available, the original flagged probe was retained.

### C. Deduplication of Probes

Due to the inclusion in the panel of several viral species with high homology, a deduplication was run using stringent hybridization settings to minimize probe removal.

### D. Specificity Check

The probe list of SEQ ID NOs: 1-28,452 was checked back against all viral sequences for specificity. Theoretical pulldown was calculated using only high stringency assumptions, 90% minimum identity over 50 bp for high stringency. The full probe pool is expected to pull down greater than 90% of all viral genomes designed against, plus all isolate sequences that went into the consensus sequences.

Additional probes include SEQ ID Nos: 28,453-213,182, which were designed using a different method. These additional probes may be included in the panel in order to more completely cover the full genomes of genetically diverse viruses such as HIV.

### Example 2. RNA Preparation and Tagmentation Enrichment of RNAs of Interest in Wastewater Samples

RNA sequencing (RNA-Seq) with next-generation sequencing (NGS) is a powerful method for discovering, profiling, and quantifying RNA transcripts. Targeted RNA-Seq analyzes expression in a focused set of genes. Enrichment enables cost-effective RNA exome analysis using sequence-specific capture of the coding regions of the transcriptome. It is ideal for low-quality samples.

This tagmentation enrichment uses on-bead tagmentation followed by a single 90-minute hybridization step to provide a rapid workflow. On-bead tagmentation features enrichment Bead-Linked Transposomes (eBLT) optimized for RNA (eBLTL) that mediate a uniform tagmentation reaction. In addition to manual preparation, RNA Preparation and Tagmentation Enrichment is designed to be compatible with liquid-handling platforms for an automated workflow, providing highly reproducible sample handling, reduced risk of human error, and less hands-on time.

A. cDNA Synthesis and Tagmentation

Wastewater is collected for evaluation of viral RNA. RNA collected from wastewater is denatured and then random hexamers are annealed. The random hexamers prime the sample for cDNA synthesis. The hexamer-primed RNA fragments are then reverse transcribed to produce first strand cDNA. Enrichment Bead-Linked Transposomes are used to tagment double-stranded cDNA.

### B. Amplification and Purification

After tagmentation, the fragments are purified and amplified to add index adapter sequences for dual indexing and P7 and P5 sequences for clustering. Next, magnetic beads are implemented to purify the tagmented library. Then the purified library is quantified and normalized.

### C. Enrichment

After normalization, the library is combined into one pool for one- or three-plex enrichment. Results are optimized for 200 ng of each library. Following quantification and normalization, the magnetic beads are implemented to capture probes hybridized to the targeted library fragments of interest. Using heated washes, nonspecific sequences bound to the beads are removed. The enriched library is then eluted from the beads. The enriched library is then amplified using a PCR program. In some embodiments, the PCR program is 14 cycles. After amplification, magnetic beads are used purify the enriched library.

### D. Evaluation

The enriched library is then evaluated using either or both of the following methods: (1) analyzing 1 μl of the enriched library with the Qubit dsDNA HS Assay kit (Illumina) to quantify library concentration (ng/μl); and/or (2) analyzing 1 μl of the enriched library with the Agilent 2100 Bioanalyzer System and a DNA 1000 Kit to qualify.

After diluting to the starting concentration depending on the sequence system, libraries are denatured and diluted to the final loading concentration. Paired-end runs are used for sequencing. The number of cycles per index read is 10, and the number of cycles per read varies depending on the sequencing system.

### Example 3. Enrichment Using a Solid Support

A solid support, such as a flowcell, is prepared for enrichment. Oligonucleotides are prepared corresponding to desired RNA, and these oligonucleotides are immobilized to a solid support. For example, oligonucleotides comprising sequences complementary to desired RNA (e.g., RNA sequences associated with viruses-of-interest) are immobilized to a solid support to allow for enrichment. A flowcell with such immobilized oligonucleotides may be termed an enrichment flowcell.

A cDNA library is prepared using the probe sets described above in Example 1 from a wastewater sample comprising RNA. Library fragments are then be added to the enrichment flowcell. Library fragments prepared from desired RNA bind to the enrichment flowcell, and the fluid that does not bind to the enrichment flowcell (comprising library fragments not prepared from desired RNA) is siphoned to a waste container. The bound library fragments are denatured, collected, and sequenced (with optional amplification before sequencing). In this way, the library that is sequenced is enriched for library fragments prepared from desired RNA.

### Example 4. Pathogen and AMR Detection in Wastewater

The Concentrating Pipette (InnovaPrep) and Nanotrap Microbiome Particles (Ceres Nanosciences) methods of microbial concentration were evaluated. In addition, four different extraction techniques were used on samples taken from different wastewater sources, including college dorms and water treatment plants in Colorado and Wisconsin. The nucleic acid was sequenced with either: (1) Shotgun metatranscriptomics performed by depleting ribosomal RNA (rRNA) using RiboZero Plus™ Microbiome (Illumina) coupled with total RNAseq library preps to profile the entire microbial content in the samples; or (2) The Urinary Pathogen ID/AMR Panel (UPIP) and a Viral Surveillance Panel (VSP) comprising viral enrichment probes described herein. UPIP targets 174 genitourinary pathogens and >3700 AMR markers while VSP targets 66 DNA and RNA viruses.

Content of concentrated wastewater samples changed over time and with the number of individuals contributing to the wastewater system. Shotgun metatranscriptomics demonstrated high levels of viruses known to be abundant in wastewater, such as hCoV-OC43 and Rotavirus A. Precision metagenomics with UPIP and VSP allowed for more in-depth strain identification as well as discovery of a greater number of less abundant pathogens, such as various noroviruses and enterovirus.

The results from these studies (not shown) provide a framework for how collecting and concentration methods can impact the variety and types of pathogens detected in samples and highlight the benefits of NGS assays that provide a comprehensive view of wastewater surveillance.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant FIGURE.

What is claimed is:

1. A method of enriching a sample for one or more target viral nucleic acids comprising the steps of:
a. providing a probe set comprising at least two nucleic acid probes complementary to one or more target viral nucleic acids, wherein the nucleic acid probes are affixed to a support;
b. capturing the one or more target viral nucleic acids on the support;
c. using the one or more captured target viral nucleic acids as a template strand to produce one or more nucleic acid duplexes immobilized on the support, wherein the one or more target viral nucleic acids hybridize to one or more probes of the probe set on the support;
d. contacting a transposase and transposon with the one or more nucleic acid duplexes under conditions wherein the one or more nucleic acid duplexes and transposon composition undergo a transposition reaction to produce one or more tagged nucleic acid duplexes, wherein the transposon composition comprises a double stranded nucleic acid molecule comprising a transferred strand and a non-transferred strand;
e. contacting the one or more tagged nucleic acid duplexes with a nucleic acid modifying enzyme under conditions to extend a 3′ end of an immobilized strand to a 5′ end of the template strand to produce one or more end-extended tagged nucleic acid duplexes;
f. amplifying the one or more end-extended tagged nucleic acid duplexes to produce a plurality of tagged nucleic acid strands;
g. contacting the plurality of tagged nucleic acid strands with a probe set to create an enriched library; and
h. amplifying the enriched library.

2. The method of claim 1, wherein the sample comprises a sample from a mammal.

3. The method of claim 1, wherein the sample comprises a blood sample, a serum sample, a tissue sample, and/or a whole blood sample.

4. The method of claim 1, comprises a freshwater sample, a wastewater sample, a saline water sample, or a combination thereof.

5. The method of claim 1, wherein the probe set is biotinylated.

6. The method of claim 1, wherein the one or more target viral nucleic acids are viral RNA molecules.

7. The method of claim 1, wherein the one or more target viral nucleic acids are genomic viral DNA or RNA molecules.

8. The method of claim 1, wherein the probe set further comprises at least two DNA probes that each hybridize to at least one target virus molecule from an adenovirus, Aichivirus, Andes virus, Anjozorobe hantavirus, Araraquara virus, Bayou virus, Bermejo virus, Black Creek Canal virus, Castelo dos Sonhos virus, Chapare virus, Chikungunya virus, Choclo virus, coxsackievirus, Crimean-Congo haemorrhagic fever virus, Dengue virus, Dobrava virus, Eastern equine encephalitis virus, Ebola virus, enterovirus, Guanarito virus, Hantaan virus, Hendra virus, hepatitis A virus, hepatitis B virus, hepatitis C virus, human coronavirus, human immunodeficiency virus 1, human immunodeficiency virus 2, human metapneumovirus, human papillomavirus, influenza A virus, influenza B virus, Japanese encephalitis virus, Juquitiba virus, KI polyomavirus Stockholm 60, Kyasanur forest disease virus, Laguna Negra virus, Lassa virus, Lechiguanas virus, Lujo virus, Machupo virus, Maciel virus, Marburg virus, Merkel cell polyomavirus, Middle East respiratory syndrome-related coronavirus, monkeypox virus, Monongahela hantavirus, Mopeia Lassa virus, Nipah virus, norovirus, Omsk hemorrhagic fever virus, orthohantavirus, parainfluenza, parechovirus, parvovirus, polyomavirus, Puumala virus, respiratory syncytial virus, rhinovirus A, rhinovirus B, rhinovirus C, Rift Valley fever, Rio Mamore virus, rotavirus A, rotavirus B, rotavirus B, rotavirus C, rotavirus H, rubella virus, Saaremaa virus, Sabia virus, salivirus, Sangassou virus, sapovirus, SARS coronavirus, Seoul virus, sin nombre virus, tick-borne encephalitis virus, torque teno virus, Tula virus, variola virus, Venezuelan equine encephalitis virus, West Nile virus, Western equine encephalomyelitis virus, yellow fever virus, and/or Zika virus.

9. The method of claim 1, wherein the probe set further comprises at least two DNA probes that each hybridize to at least one target virus molecule selected from Table 2.

10. The method of claim 1, wherein the probe set further comprises at least two DNA probes that each hybridize to at least one target virus molecule selected from Adeno-associated virus 2 (AAV2), Aichi virus 1 (AiV-A1), Alkhumra hemorrhagic fever virus (AHFV), Andes virus (ANDV), Anjozorobe virus (ANJV), Araucaria virus, Australian bat lyssavirus (ABLV), Bayou virus (BAYV), BK polyomavirus (BKPyV), Black Creek Canal virus (BCCV), Bombali virus (BOMV), Bourbon virus (BRBV), Bundibugyo virus (BDBV), Cache Valley virus (CVV), California encephalitis virus (CEV), Cedar virus (CedV), Chapare virus (CHAPV), Chikungunya virus (CHIKV), Choclo virus (CHOV), Colorado tick fever virus (CTFV), Crimean-Congo hemorrhagic fever virus (CCHFV), Crimean-Congo hemorrhagic fever virus 2 (CCHFV-2), Dengue virus (DENV), Dobrava-Belgrade virus (DOBV), Duvenhage virus (DUVV), Eastern equine encephalitis virus (EEEV), Ebola virus (EBOV), Enterovirus A, Enterovirus B, Enterovirus C, Enterovirus D, Epstein-Barr virus (EBV), European bat lyssavirus (EBLV), Ghana virus (GhV), Guanarito virus (GTOV), Hantaan virus (HTNV), Heartland virus (HRTV), Hendra virus (HeV), Henipavirus unclassified, Hepatitis A virus (HAV), Hepatitis B virus (HBV), Hepatitis C virus (HCV), Hepatitis D virus (HDV), Hepatitis E virus (HEV), Herpes simplex virus 1 (HSV1), Herpes simplex virus 2 (HSV2), Human adenovirus A, Human adenovirus B, Human adenovirus C, Human adenovirus D, Human adenovirus E, Human adenovirus F, Human adenovirus G, Human bocavirus (HBOV), Human coronavirus 229E (HCoV_229E), Human coronavirus HKU1 (HCOV_HKU1), Human coronavirus NL63 (HCoV_NL63), Human coronavirus OC43 (HCOV_OC43), Human cytomegalovirus (HCMV), Human immunodeficiency virus 1 (HIV-1), Human immunodeficiency virus 2 (HIV-2), Human metapneumovirus (HMPV), Human papillomavirus 11 (HPV11), Human papillomavirus 16 (HPV16; high-risk), Human papillomavirus 18 (HPV18; high-risk), Human papillomavirus 26 (HPV26), Human papillomavirus 31 (HPV31; high-risk), Human papillomavirus 33 (HPV33; high-risk), Human papillomavirus 35 (HPV35; high-risk), Human papillomavirus 39 (HPV39; high-risk), Human papillomavirus 40 (HPV40), Human papillomavirus 42 (HPV42), Human papillomavirus 43 (HPV43), Human papillomavirus 44 (HPV44), Human papillomavirus 45 (HPV45; high-risk), Human papillomavirus 51 (HPV51; high-risk), Human papillomavirus 52 (HPV52; high-risk), Human papillomavirus 53 (HPV53), Human papillomavirus 54 (HPV54), Human papillomavirus 56 (HPV56; high-risk), Human papillomavirus 58 (HPV58; high-risk), Human papillomavirus 59 (HPV59; high-risk), Human papillomavirus 6 (HPV6), Human papillomavirus 61 (HPV61), Human papillomavirus 66 (HPV66; high-risk), Human papillomavirus 68 (HPV68; high-risk), Human papillomavirus 69 (HPV69), Human papillomavirus 70 (HPV70), Human papillomavirus 73 (HPV73), Human papillomavirus 82 (HPV82), Human parainfluenza virus 1 (HPIV-1), Human parainfluenza virus 2 (HPIV-2), Human parainfluenza virus 3 (HPIV-3), Human parainfluenza virus 4 (HPIV-4), Human parechovirus (HPeV), Human parvovirus B19 (B19V), Human polyomavirus 6 (HPyV6), Human polyomavirus 7 (HPyV7), Human polyomavirus 9 (HPyV9), Human respiratory syncytial virus A (HRSV-A), Human respiratory syncytial virus B (HRSV-B), Influenza A virus, Influenza B virus, Influenza C virus, Isla Vista virus, Itapua virus, Jamestown Canyon virus (JCV), Japanese encephalitis virus (JEV), JC polyomavirus (JCPyV), Junin virus (JUNV), Juquitiba virus, KI polyomavirus (KIPyV), Kyasanur Forest disease virus (KFDV), La Crosse virus (LACV), Lagos bat virus (LBV), Laguna Negra virus (LANV), Langya virus, Lassa virus (LASV), LI polyomavirus (LIPyV), Lloviu virus (LLOV), Lujo virus (LUJV), Luxi virus (LUXV), Lymphocytic choriomeningitis virus (LCMV), Machupo virus (MACV), Mamastrovirus 1 (MAstV1), Mamastrovirus 6 (MAstV6), Mamastrovirus 8 (MAstV8), Mamastrovirus 9 (MAstV9), Maporal virus (MAPV), Marburg virus (MARV), Mayaro virus (MAYV), Measles virus (MV), Menangle virus (MenV), Merkel cell polyomavirus (MCPyV), Middle East respiratory syndrome-related coronavirus (MERS-COV), Mojiang virus (MojV), Mokola virus (MOKV), Monkeypox virus (MPV), Monongahela hantavirus, Muleshoe virus, Mumps virus (MuV), Murray Valley encephalitis virus (MVEV), MW polyomavirus (MWPyV), New Jersey polyomavirus (NJPyV), Nipah virus (NiV), Norovirus, Omsk hemorrhagic fever virus (OHFV), Onyong-nyong virus (ONNV), Oropouche virus (OROV), Paranoa virus, Powassan virus (POWV), Punta Toro virus (PTV), Puumala virus (PUUV), Rabies virus (RABV), Ravn virus (RAVV), Reston virus (RESTV), Rhinovirus A (RV-A), Rhinovirus B (RV-B), Rhinovirus C (RV-C), Rift Valley fever virus (RVFV), Ross River virus (RRV), Rotavirus A (RVA), Rotavirus B (RVB), Rotavirus C (RVC), Rubella virus (RuV), Sabia virus (SBAV), Salivirus A (SaV-A), Sandfly fever Sicilian virus (SFCV), Sangassou virus (SANGV), Sapovirus, Semliki Forest virus (SFV), Seoul virus (SEOV), Severe acute respiratory syndrome coronavirus (SARS-COV), Severe acute respiratory syndrome coronavirus 2 (SARS-COV-2), Severe fever with thrombocytopenia syndrome virus (SFTSV), Simian virus 40 (SV40), Sin nombre virus (SNV), Sindbis virus (SINV), Snowshoe hare virus (SSHV), Sosuga virus (SoRV), St. Louis encephalitis virus (SLEV), STL polyomavirus (STLPyV), Sudan virus (SUDV), Tacheng tick virus 2 (TcTV-2), Tahyna virus (TAHV), Tai Forest virus (TAFV), Tick-borne encephalitis virus (TBEV), Torque teno virus (TTV), Toscana virus (TOSV), Trichodysplasia spinulosa-associated polyomavirus (TSPyV), Tula virus (TULV), Usutu virus (USUV), Varicella-zoster virus (VZV), Variola virus (VARV), Venezuelan equine encephalitis virus (VEEV), West Nile virus (WNV), Western equine encephalitis virus (WEEV), WU polyomavirus (WUPyV), Yellow fever virus (YFV), and Zika virus (ZIKV).

11. The method of claim 1, wherein the at least two nucleic acid probes further comprise two or more, or five or more, or 10 or more, or 25 or more sequences, or all of the sequences selected from SEQ ID NOs: 213,288-214,878.

12. The method of claim 1, wherein the method further comprises depleting unwanted nucleic acid molecules from a nucleic acid sample.

13. The method of claim 12, wherein the depleting unwanted nucleic acid molecules comprises depleting unwanted cDNA library fragments from a library of cDNA fragments prepared from RNA, wherein the unwanted cDNA library fragments comprise those prepared from unwanted RNA sequences, further comprising:
a. preparing a solid support comprising at least one immobilized oligonucleotide, wherein each immobilized oligonucleotide comprises a nucleic acid sequence corresponding to an unwanted RNA sequence or its complement,
b. adding the library of fragments to the solid support and hybridizing the library fragments to at least one immobilized oligonucleotide to allow binding of unwanted library fragments to at least one immobilized oligonucleotide, and
c. collecting library fragments not bound to at least one immobilized oligonucleotide.

14. The method of claim 13, wherein the at least one immobilized oligonucleotide comprises a sequence comprising any one or more of SEQ ID NOs: 213,288-214,878 or its complement.

15. The method of claim 14, wherein depleting unwanted nucleic acid molecules comprises depleting off-target RNA nucleic acid molecules from a nucleic acid sample comprises:
a. contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid, wherein the off-target DNA comprises at least one small noncoding RNA chosen from RN7SK, RN7SL1, RN7SL2, RN7SL5P, RPPH1, SNORD3A;
b. contacting the DNA:RNA hybrids with a ribonuclease that degrades the RNA from the DNA:RNA hybrids, thereby degrading the off-target RNA molecules in the nucleic acid sample to form a degraded mixture;
c. separating the degraded RNA from the degraded mixture;
d. sequencing the remaining RNA from the sample;
e. evaluating the remaining RNA sequences for the presence of off-target RNA molecules from the first species, thereby determining gap sequence regions; and
f. supplementing the probe set with additional DNA probes complementary to discontiguous sequences in one or more of the gap sequence regions.

16. The method of claim 15, wherein the probe set comprises any one or more of SEQ ID NOs: 213,288-214,878, or its complement.

17. A composition comprising a probe set comprising at least one DNA probe comprising at least one sequence of SEQ ID NOs: 1-213,280, or its complement.

18. A kit comprising a probe set comprising:
a. at least one DNA probe comprising at least one sequence comprising at least one of SEQ ID NOs: 1-213,280, or its complement; and
b. a buffer.

19. The kit of claim 18, wherein the buffer is a wash buffer and/or an elution buffer.

20. The kit of claim 18, further comprising an RNA depletion buffer, a probe depletion buffer, and/or a probe removal buffer.

21. The kit of claim 18, further comprising:
a. a ribonuclease;
b. a DNase; and
c. RNA purification beads.

22. The kit of claim 21, wherein the ribonuclease is Rnase H.

23. The kit of claim 18, further comprising a nucleic acid destabilizing chemical comprising betaine, DMSO, formamide, glycerol, or a derivative thereof, or a mixture thereof.

24. The kit of claim 18, wherein the at least one DNA probe comprises 2 or more, 5 or more, 10 or more, 25 or more, 50 or more, 100 or more, 200 or more, 300 or more, 400 or more, 500 or more, 600 or more, 700 or more, 800 or more, 900 or more, 1000 or more, 1100 or more probes comprising sequences selected from SEQ ID NOs: 1-213,280, or its complement.

25. The kit of claim 18, further comprising at least one DNA probe comprising at least one sequence comprising at least one of SEQ ID NOs: 213,288-214,878.
